(19) United States
(12) Patent Application Publication
(10) Pub. No.: US 2024/0263189 A1
(43) Pub. Date: Aug. 8, 2024

(54) MUTATION OF GROWTH REGULATING FACTOR FAMILY TRANSCRIPTION FACTORS FOR ENHANCED PLANT GROWTH

(71) Applicant: Pairwise Plants Services, Inc., Durham, NC (US)

(72) Inventor: Brian Charles Wilding Crawford, Cary, NC (US)

(21) Appl. No.: 18/609,099

(22) Filed: Mar. 19, 2024

Related U.S. Application Data
(62) Division of application No. 17/126,469, filed on Dec. 18, 2020, now Pat. No. 11,965,170.
(60) Provisional application No. 62/951,593, filed on Dec. 20, 2019.

(51) Int. Cl. C12N 15/82

(52) U.S. Cl. CPC C12N 15/8262; C12N 15/8213

(57) ABSTRACT

This invention relates to compositions and methods for modifying Growth Regulating Factor (GRF) family transcription factors in plants to produce plants having improved phenotypic characteristics including increased growth. The invention further relates to plants produced using the methods and compositions of the invention.

### STATEMENT REGARDING ELECTRONIC FILING OF A SEQUENCE LISTING

A Sequence Listing in XML text format, submitted under 37 C.F.R. § 1.821-1.834, entitled 1499.16DV_ST26.xml, 522,375 bytes in size, generated on Mar. 16, 2024, and filed electronically, is provided in lieu of a paper copy. This Sequence Listing is hereby incorporated herein by reference into the specification for its disclosures.

### STATEMENT OF PRIORITY

This application is a continuation of and claims priority to U.S. application Ser. No. 18/541,481 filed Dec. 15, 2023, and the benefit, under 35 U.S.C. § 119 (c), of U.S. Provisional Application No. 62/951,593 filed on Dec. 20, 2019, the entire contents of each of which is incorporated by reference herein.

### FIELD OF THE INVENTION

This invention relates to compositions and methods for modifying growth regulating factor (GRF) family transcription factors in plants to produce plants having improved phenotypic characteristics including increased growth. The invention further relates to plants produced using the methods and compositions of the invention.

### BACKGROUND OF THE INVENTION

GRFs are land-plant-specific transcription factors that function with GRF-interacting factors (GIFs), which are found in plants and metazoans but not in fungi. The number of GRF family members is about 8-20 across the land plants. Recent studies have uncovered the functions of GRFs in other aspects of plant biology such as flowering, seed and root development, the control of growth under stress conditions, and the regulation of plant longevity.

Analysis of GRF mutants and overexpressing plants have shown that these transcription factors promote cell proliferation during leaf development. It has also been established that the mRNA of some GRFs are targeted by the microRNA miR396. For example, seven out of the nine Arabidopsis GRFs have a binding site for micro-RNA (miRNA) miR396. While initial work on the GRF transcription factor family focused on the effect of GRF mis-expression on leaf size, later work established that larger seeds can be achieved in Arabidopsis by heterogeneous expression of Brassica napus GRF2 or overexpression of AtGRF1, AtGRF2 and AtGRF5 (van Daele et al. Plant Biotechnology Journal 10:488-500 2012)).

Grain size is one of the key components of grain yield and is regulated by quantitative trait loci (QTLs) in rice. A semi-dominant QTL for grain size and weight (GS2) have been reported in rice, which encodes the transcription factor OsGRF4 (GROWTH-REGULATING FACTOR 4) and is regulated by OsmiR396. A 2 bp substitution mutation in GS2 perturbs OsmiR396-directed regulation of GS2, resulting in large and heavy grains and increased grain yield (FIG. 1).

### SUMMARY OF THE INVENTION

One aspect of the invention provides a plant or plant part thereof comprising at least one non-natural mutation in an endogenous gene encoding a Growth Regulating Factor (GRF) transcription factor, wherein the at least one non-natural mutation in the endogenous gene encoding a GRF transcription factor results in increased levels of mRNA produced by the endogenous gene.

Another aspect of the invention provides a plant cell, comprising a base editing system comprising: (a) a CRISPR-associated effector protein; (b) a cytidine deaminase or adenosine deaminase; and (c) a guide nucleic acid (gRNA, gDNA, crRNA, crDNA) having a spacer sequence with complementarity to an endogenous target gene encoding a GRF transcription factor.

A further aspect of the invention provides a plant cell comprising at least one non-naturally occurring genomic modification within a miR396 binding site of a GRF transcription factor gene that prevents or reduces binding of the miR396 to the GRF transcription factor mRNA, wherein the genomic modification is a substitution, insertion or a deletion that is introduced using an editing system that comprises a nucleic acid binding domain that binds to a target site in the GRF transcription factor gene having the nucleotide sequence of any one of SEQ ID NOs:4-18, 121-144, or 173-198.

Another aspect of the invention provides a plant or part thereof comprising a mutated endogenous GRF transcription factor gene that produces a mRNA having reduced miR396 binding, wherein the mutated endogenous GRF transcription factor gene comprises a target gene (a target nucleic acid) that comprises a sequence having at least 80% identity to any one of the nucleotide sequences of SEQ ID NOs:4, 5 and 9-18, wherein each of SEQ ID NOs:4, 5 and 9-18 comprise a sequence having at least 90% sequence identity to SEQ ID NO:1, and the target gene is modified (e.g., mutated) within the sequence having at least 90% sequence identity to SEQ ID NO:1.

Another aspect of the invention provides a plant or part thereof comprising a mutated endogenous GRF transcription factor gene that produces a mRNA having reduced miR396 binding, wherein the mutated endogenous GRF transcription factor gene comprises a target gene (a target nucleic acid) that comprises a sequence having at least 80% identity to the nucleotide sequence of SEQ ID NO:6 or SEQ ID NO:7, wherein each of SEQ ID NO:6 or SEQ ID NO:7 comprise a sequence having at least 90% sequence identity to SEQ ID NO: 2, and the target gene is modified (e.g., mutated) within the sequence having at least 90% sequence identity to SEQ ID NO: 2.

Another aspect of the invention provides a plant or part thereof comprising a mutated endogenous GRF transcription factor gene that produces a mRNA having reduced miR396 binding, wherein the mutated endogenous GRF transcription factor gene comprises a target gene (a target nucleic acid) that comprises a sequence having at least 80% identity to any one of the nucleotide sequences of SEQ ID NO:8, wherein SEQ ID NO:8 comprises a sequence having at least 90% sequence identity to SEQ ID NO: 3, and the target gene is modified (e.g., mutated) within the sequence having at least 90% sequence identity to SEQ ID NO: 3.

Another aspect of the invention provides a plant or part thereof comprising a mutated endogenous GRF transcription factor gene that produces a mRNA having reduced miR396 binding, wherein the mutated endogenous GRF transcription factor gene comprises a target gene (a target nucleic acid) that comprises a sequence having at least 80% identity to any one of the nucleotide sequences of SEQ ID NOs: 121-144, wherein each of SEQ ID NOs: 121-144 comprise a sequence having at least 90% sequence identity to any one of SEQ ID NOs:145-146, and the target gene is modified (e.g., mutated) within the sequence having at least 90% sequence identity to any one of SEQ ID NO: 145-146.

Another aspect of the invention provides a plant or part thereof comprising a mutated endogenous GRF transcription factor gene that produces a mRNA having reduced miR396 binding, wherein the mutated endogenous GRF transcription factor gene comprises a target gene (a target nucleic acid) that comprises a sequence having at least 80% identity to any one of the nucleotide sequences of SEQ ID NOs: 173-198, wherein each of SEQ ID NOs:173-198 comprise a sequence having at least 90% sequence identity to any one of SEQ ID NOs:199-202, and the target gene is modified (e.g., mutated) within the sequence having at least 90% sequence identity to any one of SEQ ID NOs: 199-202.

An additional aspect of the invention provides a plant comprising a Growth Regulating Factor (GRF) transcription factor gene that comprises a mutation in any one of the nucleotide sequences of SEQ ID NOs: 1-33 and/or comprises the nucleotide sequence of any one of SEQ ID NOs:34-41.

A further aspect of the invention provides a corn plant comprising a Growth Regulating Factor (GRF) transcription factor gene that comprises a nucleotide sequence of any one of SEQ ID NOs: 34-42.

An additional aspect of the invention provides a maize plant comprising a mutation in the miR396 binding site of a Growth Regulating Factor (GRF) transcription factor gene, the GRF transcription factor gene comprising a nucleotide sequence of any one of SEQ ID NOs:1 33.

An additional aspect of the invention provides a wheat plant comprising a mutation in the miR396 binding site of a Growth Regulating Factor (GRF) transcription factor gene, the GRF transcription factor gene comprising a nucleotide sequence of any one of SEQ ID NOs: 147-202.

A further aspect of the invention provides a soybean plant comprising a mutation in the miR396 binding site of a Growth Regulating Factor (GRF) transcription factor gene comprising a nucleotide sequence of any one of SEQ ID NOs: 97-146.

The invention further provides a method of producing/breeding a transgene-free base-edited plant, comprising: (a) crossing a plant of the invention with a transgene free plant, thereby introducing the at least one mutation, the mutation, or the modification into the plant that is transgene-free; and (b) selecting a progeny plant that comprises the at least one single nucleotide substitution but is transgene-free, thereby producing a transgene free base-edited plant.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having at least 80% sequence identity to the nucleotide sequence of any one of SEQ ID NO: 4-18, 121-144, or 173-198, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having at least 65% sequence identity to the nucleotide sequence of SEQ ID NO:9 or SEQ ID NO: 14 and comprising a miR396 binding site sequence at bp position 21 to 42 of SEQ ID NO:9 (GRF8 60 nt cDNA or SEQ ID NO:14 (GRF5 60 nt cDNA), the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of any one of SEQ ID NOs:4, 5, or 9-18 and comprising a miR396 binding site sequence at bp position 21 to 42 of any one of SEQ ID NOs:4, 5, or 9-18, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO: 6 or SEQ ID NO:7, and comprising a miR396 binding site sequence at bp position 21 to 42 of SEQ ID NO:6 or SEQ ID NO:7, the binding site sequence having at least 90% sequence identity to SEQ ID NO:2, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO: 8, and comprising a miR396 binding site sequence at bp position 21 to 42 of SEQ ID NO:8, the binding site sequence having at least 90% sequence identity to SEQ ID NO:3, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:4, and comprising a miR396 binding site sequence at bp position 21 to 42 of SEQ ID NO:4, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:5, and comprising a miR396 binding site sequence at bp position 21 to 42 of SEQ ID NO:5, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:9, and comprising a miR396 binding site sequence at bp position 21 to 42 of SEQ ID NO:9, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:10, and comprising a miR396 binding site sequence at bp position 21 to 42 of SEQ ID NO:10, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:11, and comprising a miR396 binding site sequence at bp position 21 to 42 of SEQ ID NO:1, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:12, and comprising a miR396 binding site sequence at bp position 21 to 42 of SEQ ID NO:12, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:13, and comprising a miR396 binding site sequence at bp position 21 to 42 of SEQ ID NO:13, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:14, and comprising a miR396 binding site sequence at bp position 21 to 42 of SEQ ID NO:14, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:15, and comprising a miR396 binding site sequence at bp position 21 to 42 of SEQ ID NO:15, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:16, and comprising a miR396 binding site sequence at bp position 21 to 42 of SEQ ID NO:16, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:17, and comprising a miR396 binding site sequence at bp position 21 to 42 of SEQ ID NO:17, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of SEQ ID NO:18, and comprising a miR396 binding site sequence at bp position 21 to 42 of SEQ ID NO:18, the binding site sequence having at least 90% sequence identity to SEQ ID NO:1, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of any one of SEQ ID NOs:121-144 and comprising a miR396 binding site sequence at bp position 21 to 42 of any one of SEQ ID NOs: 121-144, the binding site sequence having at least 90% sequence identity to any one of SEQ ID NOs:145-146, thereby generating an edit in the endogenous GRF transcription factor gene of the plant cell.

Another aspect of the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity to the nucleotide sequence of any one of SEQ ID NOs: 173-198 and comprising a miR396 binding site sequence at bp position 21 to 42 of any one of SEQ ID NOs: 173-198, the binding site sequence having at least 90% sequence identity to any one of SEQ ID NOs:199-202, thereby generating an edit in the endogenous GRF transcription factor gene of the plant cell.

An additional aspect of the invention provides a method for making a plant, comprising: (a) contacting a population of plant cells comprising an endogenous gene encoding a GRF transcription factor with an editing system comprising a nucleic acid binding domain that binds to a sequence having at least 80% identity to the nucleotide sequence of SEQ ID NOs: 1-3, 145, 146, or 199-202 or SEQ ID NOs:4-18, 121-144, or 173-198; (b) selecting a plant cell from said population comprising a mutation in at least one endogenous gene encoding a GRF transcription factor, wherein the mutation is a substitution of at least one nucleotide in the at least one endogenous gene, wherein the mutation reduces or eliminates the ability of miR396 to bind to a mRNA produced by the at least one endogenous gene encoding a GRF transcription factor comprising the mutation; and (c) growing the selected plant cell into a plant.

In some aspects, the invention provides a method for producing a plant or part thereof comprising at least one cell in which an endogenous GRF transcription factor gene is mutated, the method comprising contacting a target site in the GRF transcription factor gene in the plant or plant part with an editing system comprising a nucleic acid binding domain that binds to a target site in the GRF transcription factor gene having at least 80% sequence identity to the nucleotide sequence of any one of SEQ ID NOs: 1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs:19-33, 97-120, or 147-172, thereby producing a plant or part thereof comprising at least one cell having a mutation in the endogenous GRF transcription factor gene

A further aspect of the invention provides a method of producing a plant or part thereof comprising a mutated endogenous GRF transcription factor gene producing a mRNA having reduced miR396 binding, the method comprising contacting a target site in an endogenous GRF transcription factor gene with an editing system comprising a nucleic acid binding domain that binds to a target site in the GRF transcription factor gene having at least 80% sequence identity to the nucleotide sequence of any one of SEQ ID NOs:1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs:19-33, 97-120, or 147-172, thereby producing a plant or part thereof comprising a mutated endogenous GRF transcription factor gene producing a mRNA having reduced miR396 binding.

Another aspect of the invention provides a method of producing a plant or part thereof having increased growth or an increased growth rate, the method comprising contacting a target site in an endogenous GRF transcription factor gene with an editing system comprising a nucleic acid binding domain that binds to a target site in the GRF transcription factor gene having at least 80% sequence identity to the nucleotide sequence of any one of SEQ ID NOs:1-3, 145, 146, or 199-202, having at least 80% sequence identity to the nucleotide sequence of any one of SEQ ID NOs: 4-18, 121-144, or 173-198, or having at least 80% sequence identity to the nucleotide sequence of any one of SEQ ID NOs: 19-33, 97-120, or 147-172, thereby producing a plant or part thereof comprising a mutated endogenous GRF transcription factor gene that produces a mRNA having reduced miR396 binding, thereby producing a plant or part thereof having increased growth or an increased growth rate.

An additional aspect of the invention provides a guide nucleic acid (e.g., gRNA) that binds to a target site in a GRF transcription factor gene, the target site comprising any one of the nucleotide sequences of SEQ ID NOs:44-71.

A further aspect of the invention provides a system comprising the guide nucleic acid of the invention and a CRISPR-Cas effector protein that associates with the guide nucleic acid.

Another aspect of the invention provides a gene editing system comprising a CRISPR-Cas effector protein in association with a guide nucleic acid, wherein the guide nucleic acid comprises a spacer sequence that binds to a GRF transcription factor gene.

An additional aspect of the invention provides a complex comprising a CRISPR-Cas effector protein comprising a cleavage domain and a guide nucleic acid (e.g., gRNA), wherein the guide nucleic acid binds to a target site in a GRF transcription factor gene having the nucleotide sequence of any one of SEQ ID NOs: 19-33, 97-120, or 147-172, wherein the nuclease cleaves the target strand.

An additional aspect of the invention provides a complex comprising a CRISPR-Cas effector protein comprising a cleavage domain and a guide nucleic acid (e.g., gRNA), wherein the guide nucleic acid binds to a target site in a GRF transcription factor gene having the nucleotide sequence of any one of SEQ ID NOs:1-3, 145, 146, or 199-202, or SEQ ID NOs:4-18, 121-144, or 173-198, wherein the nuclease cleaves the target strand.

Another aspect of the invention provides an expression cassette comprising a (a) polynucleotide encoding CRISPR-Cas effector protein comprising a cleavage domain and (b) a guide nucleic acid that binds to a target site in a GRF transcription factor gene, wherein the guide nucleic acid comprises a spacer sequence that is complementary to and binds to a sequence having at least 80% sequence identity to at least a portion of any one of the nucleotide sequences of SEQ ID NOs: 1-3, 145, 146, or 199-202, or SEQ ID NOs:4-18, 121-144, or 173-198.

A further aspect of the invention provides a nucleic acid encoding a GRF transcription factor mRNA having a mutated miR396 binding site, wherein the mutated miR396 binding site comprises a mutation that disrupts miR396 binding and results in increased levels of the GRF transcription factor mRNA.

An additional aspect of the invention provides a corn plant or plant part thereof that comprises at least one non-natural mutation in at least one endogenous Growth Regulating Factor (GRF) transcription factor that is located in a defined chromosome interval on chromosome 1, 2, 4, 5, 6, 7, 9, and/or 10 of the corn plant, wherein the mutation disrupts the binding of miR396 to the GRF transcription factor mRNA resulting in increased levels of the GRF transcription factor mRNA.

Another aspect of the invention provides a guide nucleic acid that binds to a target nucleic acid in a GRF transcription factor in a corn plant, wherein the target nucleic acid is located in a defined chromosome interval on chromosome 1, 2, 4, 5, 6, 7, 9, and/or 10 of the corn plant.

Further provided are plants produced by the methods of the invention and comprising in their genome one or more mutated GRF transcription factor genes that produce mRNAs having a reduced ability to bind the corresponding miR396, as well as polypeptides, polynucleotides, nucleic acid constructs, expression cassettes and vectors for making a plant or part thereof this invention.

These and other aspects of the invention are set forth in more detail in the description of the invention below.

### BRIEF DESCRIPTION OF THE SEQUENCES

SEQ ID NOs:1-3 are miR396 binding site sequences in growth regulating factor (GRF) transcription factors from maize.

SEQ ID NOs:4-18 are partial cDNA sequences of GRF transcription factors from maize.

SEQ ID NOs:19-33 are full cDNA sequences of GRF transcription factors from maize.

SEQ ID NOs:34-41 are examples of mutated miR396 binding site sequences of growth regulating factor (GRF) transcription factor 6 (GRF6).

SEQ ID NOs:42-47 are GRF transcription factor proteins in, wild type (SEQ ID NO:43) and mutant proteins (SEQ ID NOs:44-48), respectively.

SEQ ID NOs:48-71 show example spacer sequences.

SEQ ID NOs:72-76, 209-213 are example adenosine deaminase sequences useful with this invention.

SEQ ID NOs:77-80, 206-208 are example cytosine deaminase amino acid sequences useful with this invention.

SEQ ID NO:81 is an exemplary uracil-DNA glycosylase inhibitor (UGI) useful with this invention.

SEQ ID NO:82-83 are exemplary regulatory sequences encoding a promoter and intron.

SEQ ID NOs:84-96 are the mature miR396 sequences from maize.

SEQ ID NOs:97-120 are full cDNA sequences of GRF transcription factors from soybean.

SEQ ID NOs:121-144 are partial cDNA sequences of GRF transcription factors from soybean.

SEQ ID NOs:145-146 are miR396 binding site sequence in growth regulating factor (GRF) transcription factors from soybean.

SEQ ID NOs:147-172 are full cDNA sequences of GRF transcription factors from wheat.

SEQ ID NOs:173-198 are partial cDNA sequences of GRF transcription factors from wheat.

SEQ ID NOs: 199-202 are miR396 binding site sequences of growth regulating factor (GRF) transcription factors from wheat.

SEQ ID NOs:203-205 provides an example of a protospacer adjacent motif position for a Type V CRISPR-Cas12a nuclease.

SEQ ID NOs:214-230 are exemplary Cas12a amino acid sequences useful with this invention.

SEQ ID NOs:231-233 are exemplary Cas12a nucleotide sequences useful with this invention.

SEQ ID NOs:234-250 are exemplary Cas12a nucleotide sequences useful with this invention.

### BRIEF DESCRIPTION OF THE DRAWINGS

FIG. 1 shows a natural QTL controlling grain size and yield in rice. A 2 bp substitution removes mir396 binding of GRF4 in rice.

FIG. 2 shows base pair and amino acid changes observed in the E0 plants from an experiment with a CRISPR-CBE (CRISPR-Cas cytosine base editor).

FIG. 3 shows an example editing window for a miR396 binding site.

FIGS. 4A-4B show example Cas9 base edits.

FIG. 5A-5B show example Cas12a (Cpf1) base edits.

FIG. 6 shows the phenotype of an E1 corn plant having a mutation in a GRF transcription factor obtained using the methods and base editing compositions as described herein.

FIG. 7 shows GRF6 and four other GRF loci targeted with 100% match to the spacer sequence. The Cas9 base editor spacer sequence (TTTCCACAGGCTTTCTTGAA SEQ ID NO:58) targets several GRF transcription factors with 100% specificity. Other GRF transcription factor targets have miss-matches (underlined nucleotides) in the spacer target sequence. The target sequence, PAM, specificity score and target gene are shown.

FIG. 8 shows Cas9 base edits in E0 plants. The wildtype GRF6 sequence is on top. The miR396 sequence is highlighted red, the spacer used for base edits blue (SEQ ID NO:58). Individual NGS reads are below for plants 7469, 7480, and 7483. Edited bases are colored. Progeny from these plants were used for phenotypic analysis in subsequent plant generations.

FIG. 9 provides a plot showing average ear length measurements from progeny of GRF4 family edits. X axis labels indicate the family of plants represented by the bar, WT indicating wild-type unedited lines, Population (Pop) 7483, Pop 9284, Pop 7480, Pop 7469 each indicating a family of progeny derived from an edited parent. Bars represent means, error bars represent 95% confidence interval around means, labels above error bars indicate number of measured samples for each family.

FIG. 10 provides a plot showing the average height from progeny of GRF4 family edits. X axis labels indicate the family of plants represented by the bar, WT indicating wild-type unedited lines, Pop 7483 and Pop 9284 each indicating a family of progeny derived from an edited parent. Bars represent means, error bars represent 95% confidence interval around means, labels above error bars indicate number of measured samples for each family.

FIG. 11 shows that Cpf1 spacer CACAGGCTTTCTTGAACGGTTGC (SEQ ID NO:55) targets only GRF6 (GRMZM2G034876) with 100% specificity. Other GRF transcription factor targets have miss-matches (red nucleotides) in the spacer target sequence. The target sequence, PAM, specificity score and target gene are shown.

FIG. 12 shows edits achieved in maize GRF6 in E0 plants from Cpf1-based deletion construct. The top sequence is the wildtype GRF6 sequence. Also shown are the miR396 target sequence and the Cpf1 spacer (SEQ ID NO:55) positions. Individual reads from E0 plants are below. The boxed reads show the 6 bp and 9 bp deletions recovered from E0 plant 13270 which is used for subsequent analyses. Numbers next to each read name are the % NGS reads recovered.

FIG. 13 provides a plot showing the average expression level of GRF4 in edited corn genotypes. X axis labels indicate the genotype of plants represented by the bar, WT/WT indicating wild-type unedited lines, 46/WT indicating lines heterozygous for a 6 bp deletion, and Δ9/WT indicating lines heterozygous for a 9 bp deletion. Y axis indicates expression of ZmGRF4 relative to endogenous control genes EF1α and Tubulin. Bars represent means, error bars represent 95% confidence interval around means, labels above error bars indicate number of measured samples for each family.

FIG. 14 provides a plot showing the expression level of a GRF in edited soybean plant compared to wild type. X axis labels indicate genotype of GRF family member (GLYMA_12 g014700) allele. Y axis indicates expression of GmGRF relative to endogenous control gene ActII. Labels above bars indicate the number of samples included for each genotype.

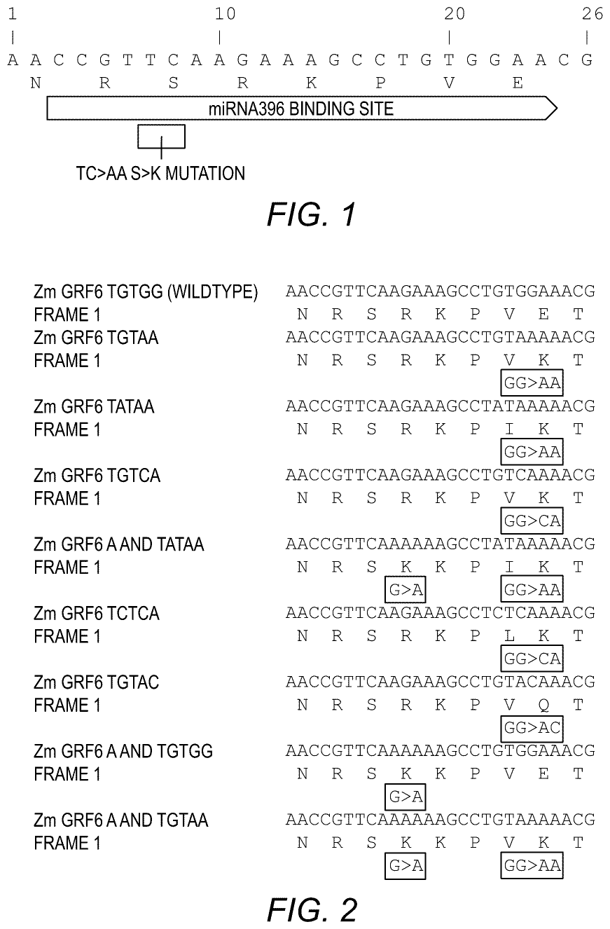

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the reference organism.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wildtype gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “bypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype. In some embodiments of this invention, the phrase “desired allele,” “target allele” or “allele of interest” refers to an allele(s) that is associated with increased yield under non-water stress conditions in a plant relative to a control plant not having the target allele or alleles.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker. For example, “a marker associated with increased yield under non-water stress conditions” refers to a marker whose presence or absence can be used to predict whether a plant will display with increased yield under non-water stress conditions.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

As used herein, “increased growth” and/or “an increased growth rate” refers to any plant trait associated with growth, for example, biomass, yield, nitrogen use efficiency (NUE), inflorescence size/weight, fruit yield, fruit quality, fruit size, seed size, seed number, foliar tissue weight, nodulation number, nodulation mass, nodulation activity, number of seed heads, number of tillers, number of flowers, number of tubers, tuber mass, bulb mass, number of seeds, total seed mass, rate of leaf emergence, rate of tiller emergence, rate of seedling emergence, length of roots, number of roots, size and/or weight of root mass, or any combination thereof. Thus, in some aspects, “increased growth” and/or “an increased growth rate” may include, but is not limited to, increased inflorescence production, increased fruit production (e.g., increased number, weight and/or size of fruit; e.g., increase number, weight, and/or size of cars for, e.g., maize), increased fruit quality, increased number, size and/or weight of roots, increased meristem size, increased seed size, increased biomass, increased leaf size, increased nitrogen use efficiency, increased disease resistance, increased height and/or increased internode length as compared to a control plant or part thereof (e.g., a plant that does not comprise a modified endogenous nucleic acid encoding a GFR transcription factor as described herein).

“Seed weight” is jointly determined by grain morphology traits such as seed length, seed width and seed thickness as well as grain filling and these traits are all governed by quantitative genetics.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a portion of a wild type CRISPR-Cas repeat sequence (e.g., a wild Type CRISR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like). In some embodiments, a nucleic acid fragment may comprise, consist essentially of or consist of about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, 550, 600, 660, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000 or more consecutive nucleotides of a nucleotide sequence encoding a GRF transcription factor.

In some embodiments, a fragment or portion may be a fragment or portion of a GRF transcription factor gene. In some embodiments, a fragment or portion may be a fragment or portion of any one of the nucleotide sequences of SEQ ID NOs:4-18, 121-144, or 173-198, wherein the fragment or portion comprises about 5, 6, 7, 8, 9, 10, 11, 12, 13, 14, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 consecutive nucleotides, or any range or value therein of any one of the nucleotide sequences of SEQ ID NOs:4-18, 121-144, or 173-198. In some embodiments, a fragment or portion may be a fragment or portion of the nucleotide sequence of any one of SEQ ID NOs:4-18, 121-144, or 173-198 may be from base pair position 5, 6, 7, 8, 9, 10, 11, 12, 13, 14, 15, 20, 21, 22, 23, 24, 25, 26, 27, 28, 29, 30, 31, 32, 33, 34, 35 to base pair position 36, 37, 38, 39, 40, 41, 42, 43, 44, 45, 46, 47, 48, 49, 50, 51, 52, 53, 54, 55, 56, 57, 58, 59, 60 of any one of SEQ ID NOs:4-18, 121-144, or 173-198. In some embodiments, an example fragment or portion of any one of the nucleotide sequences of SEQ ID NOs:4-18, 121-144, or 173-198 may be a fragment or portion comprising consecutive nucleotides from base pair position 1 to base pair position 21 or 22, from base pair position 5 to base pair position 21, 22, 23, 24, 25, 26 or 27, from base pair position 10 to base pair position 25, 26, 27, 28, 29, 30, 31, or 32, from base pair position 15 to base pair position 30, 31, 32, 33, 34, 35, 36, or 37, from base pair position 20 to base pair position 35, 36, 37, 38, 39, 40, 41, or 42, from base pair position 21 to base pair position 37, 38, 39, 40, 41, 42, 43, or 44, from base pair position 25 to base pair position 40, 41, 42, 43, 44, 45, 46, 47, or 48, from base pair position 30 to base pair position 45, 46, 47, 48, 49, 50, 51, or 52, from base pair position 35 to base pair position 50, 51, 52, 53, 54, 55, 56, or 57, from base pair position 40 to base pair position 55, 56, 57, 58, 59, 60, 61, or 62, from base pair position 42 to base pair position 57, 58, 59, 60, 61, or 62, and the like, of any one of the nucleotide sequences of SEQ ID NOs:4-18, 121-144, or 173-198 (counted from the 5′ end). In some embodiments, a fragment or portion of any one of the nucleotide sequences of SEQ ID NOs:4-18, 121-144, or 173-198 may be a fragment or portion comprising consecutive nucleotides from base pair position 20 to base pair position 42 of any one of the nucleotide sequences of SEQ ID NOs:4-18, 121-144, or 173-198 (counted from the 5′ end). In some embodiments, the at least a portion may comprise at least one nucleotide or at least two (e.g., 2, 3, 4, 5, 6, 7, 8, 9, 10, 11, 12, 13, 14, 15, 16, 17, 18, 19, 20, 21, or 22) consecutive nucleotides of any one of the nucleotide sequences of SEQ ID NOs:4-18, 121-144, or 173-198).

In some embodiments, a sequence-specific DNA binding domain may bind to one or more fragments or portions of nucleotide sequences as described herein.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400 or more consecutive amino acids of a reference polypeptide. In some embodiments, a polypeptide fragment may comprise, consist essentially of or consist of about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, 550, 600, 660, 700 or more consecutive amino acid residues of a GRF transcription factor.

As used herein with respect to nucleic acids, the term “functional fragment” refers to nucleic acid that encodes a functional fragment of a polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments, such as Southern and Northern hybridizations, are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific DNA binding domain (e.g., a sequence-specific DNA binding domain from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron may be referred to as a “promoter region” (e.g., Ubi1 promoter and intron).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (Sec, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phascolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3): 705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g, SEQ ID NO:83 and SEQ ID NO:84).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific DNA binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific DNA binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter or they may be different promoters. Thus, a polynucleotide encoding a sequence specific DNA binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

The plants or plant cultivars which are to be treated with preference in accordance with the invention include all plants which, through genetic modification, received genetic material which imparts particular advantageous useful properties (“traits”) to these plants. Examples of such properties are better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products.

Further and particularly emphasized examples of such properties are an increased resistance against animal and microbial pests, such as against insects, arachnids, nematodes, mites, slugs and snails owing, for example, to toxins formed in the plants. Among DNA sequences encoding proteins which confer properties of tolerance to such animal and microbial pests, in particular insects, mention will particularly be made of the genetic material from Bacillus thuringiensis encoding the Bt proteins widely described in the literature and well known to those skilled in the art. Mention will also be made of proteins extracted from bacteria such as Photorhabdus (WO97/17432 and WO98/08932). In particular, mention will be made of the Bt Cry or VIP proteins which include the Cry1A, CryIAb, CryIAc, CryIIA, CryIIIA, CryIIIB2, Cry9c Cry2Ab, Cry3Bb and CryIF proteins or toxic fragments thereof and also hybrids or combinations thereof, especially the Cry1F protein or hybrids derived from a CryIF protein (e.g. hybrid Cry1A-Cry1F proteins or toxic fragments thereof), the Cry1A-type proteins or toxic fragments thereof, preferably the Cry1Ac protein or hybrids derived from the Cry1Ac protein (e.g. hybrid Cry1Ab-Cry1Ac proteins) or the Cry1Ab or Bt2 protein or toxic fragments thereof, the Cry2Ac, Cry2Af or Cry2Ag proteins or toxic fragments thereof, the Cry1A.105 protein or a toxic fragment thereof, the VIP3Aa19 protein, the VIP3Aa20 protein, the VIP3A proteins produced in the COT202 or COT203 cotton events, the VIP3Aa protein or a toxic fragment thereof as described in Estruch et al. (1996), Proc Natl Acad Sci US A. 28; 93(11):5389-94, the Cry proteins as described in WO2001/47952, the insecticidal proteins from Xenorhabdus (as described in WO98/50427), Serratia (particularly from S. entomophila) or Photorhabdus species strains, such as Tc-proteins from Photorhabdus as described in WO98/08932. Also any variants or mutants of any one of these proteins differing in some amino acids (1-10, preferably 1-5) from any of the above named sequences, particularly the sequence of their toxic fragment, or which are fused to a transit peptide, such as a plastid transit peptide, or another protein or peptide, is included herein.

Another and particularly emphasized example of such properties is conferred tolerance to one or more herbicides, for example imidazolinones, sulphonylureas, glyphosate or phosphinothricin. Among DNA sequences encoding proteins (i.e., polynucleotides of interest) which confer properties of tolerance to certain herbicides on the transformed plant cells and plants, mention will be particularly be made to the bar or PAT gene or the Streptomyces coelicolor gene described in WO2009/152359 which confers tolerance to glufosinate herbicides, a gene encoding a suitable EPSPS (5-Enolpyruvylshikimat-3-phosphat-Synthase) which confers tolerance to herbicides having EPSPS as a target, especially herbicides such as glyphosate and its salts, a gene encoding glyphosate-n-acetyltransferase, or a gene encoding glyphosate oxidoreductase. Further suitable herbicide tolerance traits include at least one ALS (acetolactate synthase) inhibitor (e.g. WO2007/024782), a mutated Arabidopsis ALS/AHAS gene (e.g. U.S. Pat. No. 6,855,533), genes encoding 2,4-D-monooxygenases conferring tolerance to 2,4-D (2,4-dichlorophenoxyacetic acid) and genes encoding Dicamba monooxygenases conferring tolerance to dicamba (3,6-dichloro-2-methoxybenzoic acid).

Further examples of such properties include but are not limited to increased resistance against phytopathogenic fungi, bacteria and/or viruses owing, for example, to systemic acquired resistance (SAR), systemin, phytoalexins, elicitors and also resistance genes and correspondingly expressed proteins and toxins.

Particularly useful transgenic events in transgenic plants or plant cultivars which can be treated in accordance with the invention include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control-herbicide tolerance, deposited as PTA-6233, described in WO2005/103266 or US-A 2005-216969); Event 3006-210-23 (cotton, insect control-herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control-herbicide tolerance, deposited as ATCC PTA-11508, described in WO 11/075593); Event 43A47 (corn, insect control-herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control-herbicide tolerance, deposited as ATCC PTA 11384, described in US-A 2006-070139); Event DAS-59132 (corn, insect control-herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-I (brinjal, insect control, not deposited, described in WO 07/091277); Event Fil 17 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO98/044140); Event GHB119 (cotton, insect control-herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN1 (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait-herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control-herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MSl 1 (oilseed rape, pollination control-herbicide tolerance, deposited as ATCC PTA-850 or PTA-2485, described in WO2001/031042); Event MS8 (oilseed rape, pollination control-herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control-herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control-herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control-herbicide tolerance, not deposited, described in US-A 2005-039226 or WO2004/099447); Event VIP1034 (corn, insect control-herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession No PTA-11041) optionally stacked with event EE-GM1/LL27 or event EE-GM2/LL55 (WO2011/063413A2), event DAS-68416-4 (soybean, herbicide tolerance, ATCC Accession No PTA-10442, WO2011/066360A1), event DAS-68416-4 (soybean, herbicide tolerance, ATCC Accession No PTA-10442, WO2011/066384A1), event DP-040416-8 (corn, insect control, ATCC Accession No PTA-11508, WO2011/075593A1), event DP-043A47-3 (corn, insect control, ATCC Accession No PTA-11509, WO2011/075595A1), event DP-004114-3 (corn, insect control, ATCC Accession No PTA-11506, WO2011/084621A1), event DP-032316-8 (corn, insect control, ATCC Accession No PTA-11507, WO2011/084632A1), event MON-88302-9 (oilseed rape, herbicide tolerance, ATCC Accession No PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession N°. PTA-10296, WO2012/051199A2), event DAS-44406-6 (soybean, stacked herbicide tolerance, ATCC Accession N°. PTA-11336, WO2012/075426A1), event DAS-14536-7 (soybean, stacked herbicide tolerance, ATCC Accession N°. PTA-11335, WO2012/075429A1), event SYN-000H2-5 (soybean, herbicide tolerance, ATCC Accession N°. PTA-11226, WO2012/082548A2), event DP-061061-7 (oilseed rape, herbicide tolerance, no deposit No available, WO2012071039A1), event DP-073496-4 (oilseed rape, herbicide tolerance, no deposit No available, US2012131692), event 8264.44.06.1 (soybean, stacked herbicide tolerance, Accession No PTA-11336, WO2012075426A2), event 8291.45.36.2 (soybean, stacked herbicide tolerance, Accession N°. PTA-11335, WO2012075429A2), event SYHT0H2 (soybean, ATCC Accession N°. PTA-11226, WO2012/082548A2), event MON88701 (cotton, ATCC Accession No PTA-11754, WO2012/134808A1), event KK179-2 (alfalfa, ATCC Accession No PTA-11833, WO2013/003558A1), event pDAB8264.42.32.1 (soybean, stacked herbicide tolerance, ATCC Accession No PTA-11993, WO2013/010094A1), event MZDT09Y (corn, ATCC Accession No PTA-13025, WO2013/012775A1).

The genes/events (e.g., polynucleotides of interest), which impart a desired trait may also be present in combinations with one another in a transgenic plant. Examples of transgenic plants include, but are not limited to, crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails, as well as the increased resistance of the plants to one or more herbicides.

Commercially available examples of such plants, plant parts or plant seeds that may be treated with preference in accordance with the invention include commercial products, such as plant seeds, sold or distributed under the GENUITY®, DROUGHTGARD®, SMARTSTAX®, RIB COMPLETE®, ROUNDUP READY®, VT DOUBLE PRO®, VT TRIPLE PRO®, BOLLGARD II®, ROUNDUP READY 2 YIELD®, YIELDGARD®, ROUNDUP READY® 2 XTEN^{D}™, INTACTA RR2 PRO®, VISTIVE GOLD®, and/or XTENDFLEX™ trade names.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific DNA binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific DNA binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g. expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific DNA binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby sequence-specific DNA binding protein, the reverse transcriptase and the deaminase are expressed and the sequence-specific DNA binding protein binds to the target nucleic acid, and the reverse transcriptase and/or deaminase may be either fused to the sequence-specific DNA binding protein or recruited to the sequence-specific DNA binding protein (via, for example, a peptide tag fused to the sequence-specific DNA binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromosomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

GRF transcription factors are involved in growth promotion in plants. The expression of GRF transcription factors can increase growth when they are ectopically or over expressed. One common mechanism to repress expression of the GRF transcription factors is through the microRNA, miR396. MicroRNAs are small non-coding RNA molecule found in plants, animals and some viruses, which function in RNA silencing and post-transcriptional regulation of gene expression. miRNAs function via base-pairing with complementary sequences within mRNA molecules. The present inventors have discovered that by changing the binding site of miRNA binding in a target mRNA it is possible to remove the ability of the miRNA to post-transcriptionally regulate the target genes expression.

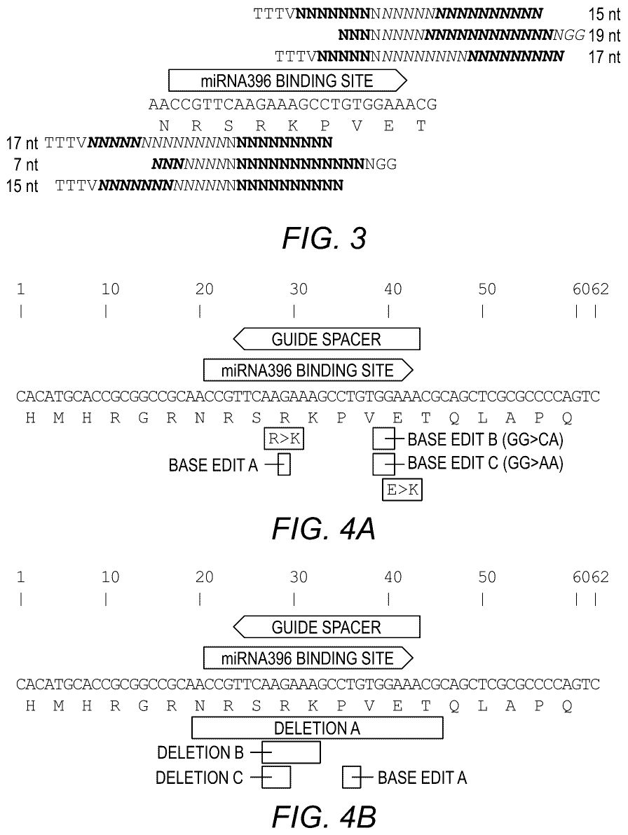

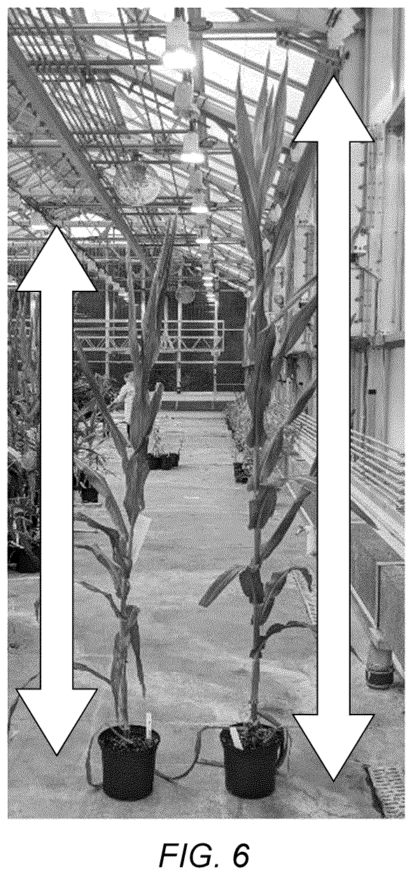

In some embodiments, a plant or plant part thereof is provided, the plant or part there of comprising at least one non-natural mutation in at least one gene encoding an endogenous Growth Regulating Factor (GRF) transcription factor, wherein the mutation disrupts the binding of miR396 to the mRNA that is produced by the at least one gene encoding the endogenous GRF transcription factor resulting in increased levels of the mRNA. In some embodiments, a mutation that disrupts the binding of miR396 to the GRF transcription factor mRNA and results in increased levels of the mRNA as described herein is a stable mutation, optionally wherein the mutation is in the miR396 binding site of the mRNA encoded by the GRF transcription factor gene (see, e.g., FIG. 3). In some embodiments, at least one non-natural mutation in at least one gene encoding an GRF transcription factor is a semi-dominant mutation and/or a hypomorphic mutation. In some embodiments, the endogenous GRF transcription factor that is mutated may include, but is not limited to, a GRF1, GRF2, GRF3, GRF4, GRF5, GRF6, GRF7, GRF8, GRF9, GRF10, GRF11, GRF12, GRF13, GRF14, GRF15, GRF16, GRF17, GRF18, GRF19 or a GRF20. The plant may be any plant as described herein. In some embodiments, the plant may be a wheat plant or a corn plant (see, e.g., Table 1). In some embodiments, GRF transcription factor may be from corn and may comprise a GRF6, GRF2, GRF1, GRF14, GRF15, GRF8, GRF4, GRF12, GRF10, GRF11, GRF5, GRF3, GRF9, GRF13, or a GRF7. In some embodiments, the corn plant may comprise a short stature/semi-dwarf phenotype (see, e.g., FIG. 6). In some embodiments, the at least one non-natural mutation in at least one gene encoding an endogenous GRF transcription factor in a wheat plant may be in the A genome, B genome, or D genome, or any combination thereof.

In some embodiments, a GRF transcription factor gene produces an mRNA that binds a corresponding miR396. In some embodiments, the corresponding miR396 may be a mir396a, a miR396b, a mir396c, a miR396d, a mir396c, a miR396f, a mir396g, and/or a miR396h (see, e.g., SEQ ID NOs:84-96). In some embodiments, a GRF transcription factor mRNA binds a miR396a. In some embodiments, a GRF transcription factor mRNA binds a miR396b. In some embodiments, reduced miR396 binding to a GRF transcription factor mRNA may be measured by measuring the levels of the GRF transcription factor mRNA.

In some embodiments, the at least one non-natural mutation in at least one endogenous GRF transcription factor gene may be a base substitution, a deletion and/or an insertion. In some embodiments, the at least one mutation may be a synonymous mutation in the portion of the GRF transcription factor gene that produces the miRNA binding site of the GRF transcription factor mRNA. In some embodiments, at least one non-natural mutation in at least one gene encoding an GRF transcription factor is a semi-dominant mutation and/or a hypomorphic mutation. In some embodiments, the portion of the GRF transcription factor gene that produces the miR396 binding site in the GRF transcription factor mRNA encodes a portion of the endogenous GRF transcription factor polypeptide, wherein the portion of the endogenous GRF transcription factor polypeptide comprises the amino acid sequence of NRSRKPVET (SEQ ID NO: 42). In some embodiments, the at least one mutation may be a non-synonymous mutation in the nucleotide sequence that encodes a portion of the endogenous GRF transcription factor amino acid sequence, said portion having the amino acid sequence of NRSRKPVET (SEQ ID NO:42). In some embodiments, the at least one mutation may be an in-frame deletion in the nucleotide sequence that encodes a portion of the endogenous GRF transcription factor amino acid sequence, said portion having the amino acid sequence of NRSRKPVET (SEQ ID NO:42). Thus, in some embodiments the invention provides a plant cell comprising a stable and targeted (e.g., non-natural) single nucleotide substitution in an endogenous gene encoding a GRF transcription factor. In some embodiments, a mutation in an endogenous gene encoding a GRF transcription factor does not result in a mutation in the polypeptide sequence of the GRF transcription factor. In some embodiments, a mutation in an endogenous gene encoding a GRF transcription factor produces a polypeptide comprising a mutation in the polypeptide sequence of the GRF transcription factor. In some embodiments, a mutated GRF transcription factor polypeptide may comprise the amino acid sequence of NRSRKPVKT (SEQ ID NO:43), NRSRKPIKT (SEQ ID NO:44), NRSKKPIKT (SEQ ID NO:45), NRSRKPVQT (SEQ ID NO:46), and/or NRSKKPVKT (SEQ ID NO:47).

In some embodiments, an endogenous GRF transcription factor gene that produces an mRNA having a miR396 binding site comprises: (a) a nucleotide sequence having at least 90% sequence identity to any one of the nucleotide sequences of SEQ ID NOs: 1-3, 145, 146, or 199-202; (b) a nucleotide sequence having at least 95% identity (e.g., about 95, 96, 70, 98, 99, or 100%) to any one of the nucleotide sequences of SEQ ID NOs:1-3, 145, 146, or 199-202; or (c) the nucleotide sequence set forth in any one of the nucleotide sequences of SEQ ID NOs:1-3, 145, 146, or 199-202. In some embodiments, a base substitution can be a substitution of any base to any different base, e.g., to an A, T, C, or G. In some embodiments, the endogenous GRF transcription factor gene may comprise a mutation in at least one of positions 11, 19, 21 and/or 22 of any one of the nucleotide sequences of SEQ ID NOs: 1-3, 145, 146, or 199-202. In some embodiments, a mutation at position 11 of any one of the nucleotide sequences of SEQ ID NOs: 1-3, 145, 146, or 199-202 may be from G to A, T, or C (e.g., G>A, G>T, G>C), a mutation at position 19 of any one of the nucleotide sequences of SEQ ID NOs:1-3, 145, 146, or 199-202 may be from G to A, T, or C, a mutation at position 21 of any one of the nucleotide sequences of SEQ ID NOs:1-3, 145, 146, or 199-202 may be from G to A, T, or C and/or a mutation at position 22 of any one of the nucleotide sequences of SEQ ID NOs: 1-3, 145, 146, or 199-202 is from G to A, T, or C. In some embodiments, a mutation at position 11 of any one of the nucleotide sequences of SEQ ID NOs:1-3, 145, 146, or 199-202 may be G>A. In some embodiments, a mutation at position 19 of any one of the nucleotide sequences of SEQ ID NOs: 1-3, 145, 146, or 199-202 may be G>A or G>C. In some embodiments, a mutation at position 21 of any one of the nucleotide sequences of SEQ ID NOs: 1-3, 145, 146, or 199-202 may be G>A or G>C. In some embodiments, a mutation at position 22 of any one of the nucleotide sequences of SEQ ID NOs: 1-3, 145, 146, or 199-202 may be G>A or G>C.

A mutation in an endogenous GRF transcription factor gene that comprises the nucleotide sequence of any one of SEQ ID NOs: 1-3, 145, 146, or 199-202 may or may not produce a change in the amino acid sequence. Thus, a mutation to an endogenous GRF transcription factor gene comprising the nucleotide sequence of any one of SEQ ID NOs:1-3 may result in the same amino acid sequence of NRSRKPVET (SEQ ID NO:42). In some embodiments, a mutation to endogenous GRF transcription factor gene comprising the nucleotide sequence of any one of SEQ ID NOs:1-3 may result in an amino acid sequence that can include, but is not limited to, the amino acid sequence of any one of NRSRKPVKT (SEQ ID NO:43), NRSRKPIKT (SEQ ID NO:44), NRSKKPIKT (SEQ ID NO:45), NRSRKPVQT (SEQ ID NO:46), and/or NRSKKPVKT (SEQ ID NO:47).

In some embodiments, the invention provides a plant cell, the plant cell comprising a base editing system comprising: (a) a sequence-specific binding domain (e.g., a CRISPR-associated effector protein); (b) a cytidine deaminase or adenosine deaminase; and (c) a guide nucleic acid (gRNA) having a spacer sequence with complementarity to an endogenous target gene encoding a GRF transcription factor. In some embodiments, an endogenous target gene encoding a GRF transcription factor may comprise at least about 90% sequence identity (e.g., about 90, 91, 92, 93, 94, 95, 96, 70, 98, 99, or 100%) to any one of the nucleotide sequences of SEQ ID NOs:1-3, 145, 146, or 199-202. In some embodiments, a guide nucleic acid may comprise a spacer sequence having the nucleotide sequence of, for example, any one of SEQ ID NOs:48-71.

In some embodiments, a guide nucleic acid useful with this invention may comprise spacer that is complementary to a fragment or portion of the nucleotide sequence of any one of SEQ ID NOs:4-18, 121-144, or 173-198, wherein the fragment or portion of any one of SEQ ID NOs:4-18, 121-144, or 173-198 may be from base pair position 5, 6, 7, 8, 9, 10, 11, 12, 13, 14, 15, 20, 21, 22, 23, 24, 25, 26, 27, 28, 29, 30, 31, 32, 33, 34, 35 to base pair position 36, 37, 38, 39, 40, 41, 42, 43, 44, 45, 46, 47, 48, 49, 50, 51, 52, 53, 54, 55, 56, 57, 58, 59, 60 of any one of SEQ ID NOs:4-18, 121-144, or 173-198. In some embodiments, a fragment or portion of any one of SEQ ID NOs:4-18, 121-144, or 173-198 to which a spacer for a guide nucleic acid may be complementary may comprise consecutive nucleotides from base pair position 1 to base pair position 21 or 22, from base pair position 5 to base pair position 21, 22, 23, 24, 25, 26 or 27, from base pair position 10 to base pair position 25, 26, 27, 28, 29, 30, 31, or 32, from base pair position 15 to base pair position 30, 31, 32, 33, 34, 35, 36, or 37, from base pair position 20 to base pair position 35, 36, 37, 38, 39, 40, 41, or 42, from base pair position 21 to base pair position 37, 38, 39, 40, 41, 42, 43, or 44, from base pair position 25 to base pair position 40, 41, 42, 43, 44, 45, 46, 47, or 48, from base pair position 30 to base pair position 45, 46, 47, 48, 49, 50, 51, or 52, from base pair position 35 to base pair position 50, 51, 52, 53, 54, 55, 56, or 57, from base pair position 40 to base pair position 55, 56, 57, 58, 59, 60, 61, or 62, from base pair position 42 to base pair position 57, 58, 59, 60, 61, or 62, and the like, of any one of the nucleotide sequences of SEQ ID NOs:4-18, 121-144, or 173-198 (counted from the 5′ end). In some embodiments, a spacer sequence for a guide nucleic acid may be complementary to consecutive nucleotides from base pair position 20 to base pair position 42 of any one of the nucleotide sequences of SEQ ID NOs:4-18, 121-144, or 173-198 (counted from the 5′ end). In some embodiments, a spacer sequence for a guide nucleic acid may comprise a spacer sequence having the nucleotide sequence of any one of SEQ ID NOs:48-71.

In some embodiments, a plant part or plant cell produced by the methods of this invention may be regenerated to produce a plant comprising a stable mutation in at least one gene encoding an endogenous GRF transcription factor, wherein the mutation results in reduced binding of a corresponding miR396 to the mRNA that is produced by the endogenous GRF transcription factor gene, optionally wherein the mutation is a semi-dominant mutation and/or a hypomorphic mutation.

In some embodiments, the invention further provides a plant cell comprising at least one non-naturally occurring genomic modification within a miR396 binding site of a GRF transcription factor gene that prevents or reduces binding of the miR396 to the GRF transcription factor mRNA transcribed from the GRF transcription factor gene comprising the at least one non-natural genomic modification, wherein the genomic modification is a substitution, insertion or a deletion that is introduced using an editing system that comprises a nucleic acid binding domain that binds to a target site in the GRF transcription factor gene having the nucleotide sequence of any one of SEQ ID NOs:4-18, 121-144, or 173-198.

In some embodiments, the invention provides a plant or part thereof comprising a mutated endogenous GRF transcription factor gene that produces an mRNA having reduced miR396 binding, wherein the mutated endogenous GRF transcription factor gene comprises a target gene (a target nucleic acid) that comprises a sequence having at least 80% sequence identity (e.g., about 80, 81, 82, 83, 84, 85, 86, 87, 88, 89, 90, 91, 92, 93, 94, 95, 96, 70, 98, 99, or 100%) to any one of the nucleotide sequences of SEQ ID NOs:4, 5 or 9-18, wherein each of SEQ ID NOs:4, 5 or 9-18 comprise a sequence having at least 90% sequence identity (e.g., about 90, 91, 92, 93, 94, 95, 96, 70, 98, 99, or 100%) to SEQ ID NO:1, and the target gene is modified (e.g., mutated) within the sequence having at least 90% sequence identity to SEQ ID NO:1. In some embodiments, the modification (e.g., mutation) within the sequence having at least 90% sequence identity to the nucleotide sequence of SEQ ID NO:1 may be introduced using an editing system that comprises a sequence-specific DNA binding domain that binds to a target site within a nucleotide sequence having at least 80% identity to any one of the nucleotide sequences of SEQ ID NOs:4, 5 or 9-18. In some embodiments, the modification within the sequence having at least 90% sequence identity to the nucleotide sequence of SEQ ID NO:1 may be introduced using an editing system that comprises a sequence-specific DNA binding domain that binds to a target site within the nucleotide sequence of any one of SEQ ID NOs:19-33.

In some embodiments, a plant or part thereof is provided, the plant or plant part thereof comprising a mutated endogenous GRF transcription factor gene that produces a mRNA having reduced miR396 binding and resulting in increased levels of mRNA produced by the endogenous GRF transcription factor gene, wherein the mutated endogenous GRF transcription factor gene comprises a target gene (a nucleic acid) that comprises a sequence having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:6 or SEQ ID NO:7, wherein each of the nucleotide sequence of SEQ ID NO:6 or SEQ ID NO:7 comprise a sequence having at least 90% sequence identity to the nucleotide sequence of SEQ ID NO: 2, and the target gene is modified (e.g., mutated) within the sequence having at least 90% sequence identity (e.g., about 90, 91, 92, 93, 94, 95, 96, 70, 98, 99, or 100%) to the nucleotide sequence of SEQ ID NO: 2. In some embodiments, the modification (e.g., mutation) within the sequence having at least 90% sequence identity to SEQ ID NO: 2 may be introduced using an editing system that comprises a nucleic acid binding domain that binds to a target site within the sequence having at least 80% identity SEQ ID NO:6 or SEQ ID NO:7. In some embodiments, the modification/mutation within the sequence having at least 90% sequence identity to SEQ ID NO: 2 may be introduced using an editing system that comprises a sequence-specific DNA binding domain that binds to a target site within the nucleotide sequence of any one of SEQ ID NO:21 or SEQ ID NO:22. In some embodiments, the mutation that is introduced by the editing system may be in at least one gene encoding an GRF transcription factor is a semi-dominant mutation and/or a hypomorphic mutation.

In some embodiments, a plant or part thereof is provided comprising at least one non-natural mutation in an endogenous gene encoding a Growth Regulating Factor (GRF) transcription factor, wherein the at least one non-natural mutation in the endogenous gene encoding a GRF transcription factor results in increased levels of mRNA produced by the endogenous gene. In some embodiments, the at least one non-natural mutation in the endogenous gene encoding a GRF transcription factor results in increased levels of mRNA produced by the endogenous gene and disrupts binding of miR396 to the mRNA produced by the GRF transcription factor gene.

In some embodiments, a plant or part thereof is provided, the plant or plant part thereof comprising a mutated endogenous GRF transcription factor gene that produces a mRNA having reduced miR396 binding and resulting in increased levels of GRF transcription factor mRNA, wherein the mutated endogenous GRF transcription factor gene comprises a target gene (a nucleic acid) that comprises a sequence having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:8, wherein the nucleotide sequence of SEQ ID NO:8 comprises a sequence having at least 90% sequence identity (e.g., about 90, 91, 92, 93, 94, 95, 96, 70, 98, 99, or 100%) to the nucleotide sequence of SEQ ID NO: 3, and the target gene is modified (e.g., mutated) within the sequence having at least 90% sequence identity to the nucleotide sequence of SEQ ID NO: 3. In some embodiments, the modification (e.g., mutation) within the sequence having at least 90% sequence identity to SEQ ID NO:3 may be introduced using an editing system that comprises a nucleic acid binding domain that binds to a target site within the sequence having at least 80% identity to SEQ ID NO:8. In some embodiments, the modification within the sequence having at least 90% sequence identity to SEQ ID NO:3 may be introduced using an editing system that comprises a sequence-specific DNA binding domain that binds to a target site within the nucleotide sequence of any one of SEQ ID NO:23.

In some embodiments, the invention provides a plant or part thereof comprising a mutated endogenous GRF transcription factor gene that produces a mRNA having reduced miR396 binding, wherein the mutated endogenous GRF transcription factor gene comprises a target gene (a nucleic acid) that comprises a sequence having at least 80% sequence identity (e.g., about 80, 81, 82, 83, 84, 85, 86, 87, 88, 89, 90, 91, 92, 93, 94, 95, 96, 70, 98, 99, or 100%) to any one of the nucleotide sequences of SEQ ID NOs:121-144, wherein each of SEQ ID NOs:121-144 comprise a sequence having at least 90% sequence identity (e.g., about 90, 91, 92, 93, 94, 95, 96, 70, 98, 99, or 100%) to any one of SEQ ID NOs: 145-146, and the target gene is modified (e.g., mutated) within the sequence having at least 90% sequence identity to any one of SEQ ID NOs: 145-146. In some embodiments, the modification (e.g., mutation) within the sequence having at least 90% sequence identity to any one of the nucleotide sequence of SEQ ID NOs:145-146 may be introduced using an editing system that comprises a sequence-specific DNA binding domain that binds to a target site within a nucleotide sequence having at least 80% identity to any one of the nucleotide sequences of SEQ ID NOs: 121-144. In some embodiments, the modification within the sequence having at least 90% sequence identity to any one of the nucleotide sequence of SEQ ID NOs:145-146 may be introduced using an editing system that comprises a sequence-specific DNA binding domain that binds to a target site within the nucleotide sequence of any one of SEQ ID NOs: 121-144.

In some embodiments, the invention provides a plant or part thereof comprising a mutated endogenous GRF transcription factor gene that produces a mRNA having reduced miR396 binding, wherein the mutated endogenous GRF transcription factor gene comprises a target gene (a nucleic acid) that comprises a sequence having at least 80% sequence identity (e.g., about 80, 81, 82, 83, 84, 85, 86, 87, 88, 89, 90, 91, 92, 93, 94, 95, 96, 70, 98, 99, or 100%) to any one of the nucleotide sequences of SEQ ID NOs:173-198, wherein each of SEQ ID NOs:173-198 comprise a sequence having at least 90% sequence identity (e.g., about 90, 91, 92, 93, 94, 95, 96, 70, 98, 99, or 100%) to any one of SEQ ID NOs: 199-202, and the target gene is modified (e.g., mutated) within the sequence having at least 90% sequence identity to any one of SEQ ID NOs:199-202, and the target gene is modified (e.g., mutated) within the sequence having at least 90% sequence identity to any one of SEQ ID NOs: 199-202. In some embodiments, the modification (e.g., mutation) within the sequence having at least 90% sequence identity to any one of the nucleotide sequence of SEQ ID NOs: 199-202 may be introduced using an editing system that comprises a sequence-specific DNA binding domain that binds to a target site within a nucleotide sequence having at least 80% identity to any one of the nucleotide sequences of SEQ ID NOs:173-198. In some embodiments, the modification within the sequence having at least 90% sequence identity to any one of the nucleotide sequence of SEQ ID NOs: 199-202 may be introduced using an editing system that comprises a sequence-specific DNA binding domain that binds to a target site within the nucleotide sequence of any one of SEQ ID NOs:174-198.

An additional aspect of the invention provides a plant comprising a Growth Regulating Factor (GRF) transcription factor gene that comprises a mutation in any one of the nucleotide sequences of any one of SEQ ID NOs:1-33 or 97-202. In some embodiments, the invention provides a plant comprising a mutated Growth Regulating Factor (GRF) transcription factor gene that comprises a nucleotide sequence of any one of SEQ ID NOs:34-41. In some embodiments, the mutated GRF transcription factor comprises a semi-dominant mutation and/or a hypomorphic mutation.

In some embodiments, the invention provides a corn plant comprising a Growth Regulating Factor (GRF) transcription factor gene that comprises a nucleotide sequence of any one of SEQ ID NOs:34-41.

In some embodiments, the invention provides a wheat plant comprising a Growth Regulating Factor (GRF) transcription factor gene that produces a mRNA having a mutated miR396 binding site such that the binding of miR396 to the miR396 binding site of the GRF transcription factor mRNA is reduced. In some embodiments, the invention provides a wheat plant having a mutation in the miR396 binding site of a mRNA produced by a Growth Regulating Factor (GRF) transcription factor gene that comprises a nucleotide sequence of any one of SEQ ID NOs: 147-198. In some embodiments, the Growth Regulating Factor (GRF) transcription factor gene that comprises a nucleotide sequence of any one of SEQ ID NOs: 147-198 is in the A genome, the B genome, the D genome, or any combination thereof, of a wheat plant.

In some embodiments, the invention provides a wheat plant comprising a Growth Regulating Factor (GRF) transcription factor gene that produces a mRNA having a mutated miR396 binding site such that the binding of miR396 to the miR396 binding site of the GRF transcription factor mRNA is reduced. In some embodiments, the invention provides a soybean plant having a mutation in the miR396 binding site of a mRNA produced by a Growth Regulating Factor (GRF) transcription factor that comprises a nucleotide sequence of any one of SEQ ID NOs:97-144. In some embodiments, the Growth Regulating Factor (GRF) transcription factor that comprises a nucleotide sequence of any one of SEQ ID NOs:97-144 is in the A genome, the B genome, the D genome, or any combination thereof, of a wheat plant.

In some embodiments, the invention provides a corn plant or plant part thereof comprising at least one non-natural mutation in at least one endogenous Growth Regulating Factor (GRF) transcription factor that is located in: (a) a chromosome interval defined by and including base pair (bp) position 211773221 to base pair position 211774543 on chromosome 5 (e.g., Gene ID No. GRMZM5G893117), optionally a chromosome interval defined by and including base pair (bp) position 211773897 to base pair position 211773957; (b) a chromosome interval defined by and including base pair (bp) position 200344009 to base pair position 200348833 on chromosome 5 (e.g., Gene ID No. GRMZM2G034876), optionally a chromosome interval defined by and including base pair (bp) position 200346611 to base pair position 200346671; (c) a chromosome interval defined by and including base pair (bp) position 13817620 to base pair position 13822440 on chromosome 5 (e.g., Gene ID No. GRMZM2G033612), optionally a chromosome interval defined by and including base pair (bp) position 138197933 to base pair position 13819853; (d) a chromosome interval defined by and including base pair (bp) position 177151736 to base pair position 177154443 on chromosome 4 (e.g., Gene ID No. GRMZM2G105335), optionally a chromosome interval defined by and including base pair (bp) position 177152804 to base pair position 177152864; (c) a chromosome interval defined by and including base pair (bp) position 257245490 to base pair position 257249295 on chromosome 1 (e.g., Gene ID No. GRMZM2G018414), optionally a chromosome interval defined by and including base pair (bp) position 257247225 to base pair position 257247285; (f) a chromosome interval defined by and including base pair (bp) position 25722859 to base pair position 25725873 on chromosome 9 (e.g., Gene ID No. GRMZM2G119359), optionally a chromosome interval defined by and including base pair (bp) position 25723770 to base pair position 25723830; (g) a chromosome interval defined by and including base pair (bp) position 60351912 to base pair position 60356302 on chromosome 6 (e.g., Gene ID No. GRMZM2G098594), optionally a chromosome interval defined by and including base pair (bp) position 60354489 to base pair position 60354549; (h) a chromosome interval defined by and including base pair (bp) position 196192921 to base pair position 196194872 on chromosome 5, optionally a chromosome interval defined by and including base pair (bp) position 196194175 to base pair position 196194235; (i) a chromosome interval defined by and including base pair (bp) position 140393394 to base pair position 140398705 on chromosome 10 (e.g., Gene ID No. GRMZM2G129147), optionally a chromosome interval defined by and including base pair (bp) position 140396272 to base pair position 140396332; (j) a chromosome interval defined by and including base pair (bp) position 155831622 to base pair position 155833517 on chromosome 4 (e.g., Gene ID No. GRMZM2G124566), optionally a chromosome interval defined by and including base pair (bp) position 155832749 to base pair position 155832809;
(k) a chromosome interval defined by and including base pair (bp) position 12201898 to base pair position 12208052 on chromosome 2 (e.g., Gene ID No. GRMZM2G041223), optionally a chromosome interval defined by and including base pair (bp) position 12204599 to base pair position 12204659; (1) a chromosome interval defined by and including base pair (bp) position 9818327 to base pair position 9820186 on chromosome 9 (e.g., Gene ID No. GRMZM2G067743), optionally a chromosome interval defined by and including base pair (bp) position 9819224 to base pair position 9819284; (m) a chromosome interval defined by and including base pair (bp) position 108477686 to base pair position 108480099 on chromosome 6 (e.g., Gene ID No. GRMZM5G850129), optionally a chromosome interval defined by and including base pair (bp) position 108478747 to base pair position 108478807; (n) a chromosome interval defined by and including base pair (bp) position 225827712 to base pair position 225832487 on chromosome 2 (e.g., Gene ID No. GRMZM2G099862), optionally a chromosome interval defined by and including base pair (bp) position 225830099 to base pair position 225830159; (o) a chromosome interval defined by and including base pair (bp) position 272414778 to base pair position 272420567 on chromosome 1 (e.g., Gene ID No. GRMZM2G178261); (p) a chromosome interval defined by and including base pair (bp) position 199377455 to base pair position 199381799 on chromosome 2; (q) a chromosome interval defined by and including base pair (bp) position 145016279 to base pair position 145019899 on chromosome 7; and/or (r) a chromosome interval defined by and including base pair (bp) position 8708791 to base pair position 8711617 on chromosome 5 (e.g., Gene ID No. GRMZM5G853392), wherein the mutation results in increased GRF transcription factor mRNA, and optionally, disrupts the binding of miR396 to the GRF transcription factor, and the chromosomal intervals of (a) to (r) correspond to the reference maize genome of B73.

In some embodiments, a corn plant may comprise a mutation on chromosome 5 (a) bp position 211773927, (b) bp position 200346641, (c) bp position 13819823, and (d) bp position 196194205, wherein the chromosomal position of (a) to (c) correspond to the reference maize genome of B73.

In some embodiments, a corn plant may comprise a mutation on chromosome 4 (a) bp position 177152834, and/or (b) bp position 155832779, wherein the chromosomal position of (a) and (b) correspond to the reference maize genome of B73.

In some embodiments, a corn plant may comprise a mutation on chromosome 1, bp position 257247255, wherein the chromosomal position of 257247255 corresponds to the reference maize genome of B73.

In some embodiments, a corn plant may comprise a mutation on chromosome 9 (a) bp position 25723800 and/or (b) bp position 9819254, wherein the chromosomal position of (a) and (b) correspond to the reference maize genome of B73.

In some embodiments, a corn plant may comprise a mutation on chromosome 6 (a) bp position 60354519 and/or (b) bp position 108478777, wherein the chromosomal position of (a) and (b) correspond to the reference maize genome of B73.

In some embodiments, a corn plant may comprise a mutation on chromosome 10, bp position 140396302, wherein the chromosomal position of 140396302 corresponds to the reference maize genome of B73.

In some embodiments, a corn plant may comprise a mutation on chromosome 2 (a) bp position 12204629, and/or (b) bp position 225830129, wherein the chromosomal position of (a) and (b) correspond to the reference maize genome of B73.

With regard to corn (Zea mays), markers of the present invention are described herein with respect to the positions of marker loci in the B73 corn genome “B73 RefGen_v3” (assembly aka B73 RefGen_v3, AGPv3) at the MaizeGDB internet resource (maizegdb.org/assembly).

The present invention provides methods and compositions for modifying GRF transcription factors involved in plant growth and development, in particular those GRF transcription factors that regulate biomass, yield, inflorescence size/weight, fruit yield, fruit quality, fruit size, seed size, seed number, foliar tissue weight, nodulation number, nodulation mass, nodulation activity, number of seed heads, number of tillers, number of flowers, number of tubers, tuber mass, bulb mass, number of seeds, total seed mass, rate of leaf emergence, rate of tiller emergence, rate of seedling emergence, length of roots, number of roots, size and/or weight of root mass, or any combination of these characteristics. Thus, in some embodiments of the invention, a plant comprising a mutated endogenous GRF transcription factor gene having reduced miR396 binding, may exhibit at least one of the following phenotypes of increased meristem size, increased seed size, increased biomass, increased leaf size, increased root size, increased nitrogen use efficiency, increased disease resistance, increased height and/or increased internode length as compared to a control plant that does not comprise the mutated endogenous GRF transcription factor gene having reduced miR396 binding.

In some embodiments, the present invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one of SEQ ID NOs:4-18, 121-144, or 173-198, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having at least 65% sequence identity (e.g., about 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 70, 98, 99, or 100%) to the nucleotide sequence of SEQ ID NO:9 or SEQ ID NO:14 and comprising a miR396 binding site sequence at bp position 21 to 42 of SEQ ID NO:9 (GRF8 60 nt cDNA or SEQ ID NO:14 (GRF5 60 nt cDNA), the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one of SEQ ID NOs:4, 5, or 9-18 and comprising a miR396 binding site sequence at bp position 21 to 42 of any one of SEQ ID NOs:4, 5, or 9-18, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, the present invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:6 or SEQ ID NO:7, and comprising a miR396 binding site sequence at bp position 21 to 42 of SEQ ID NO:6 or SEQ ID NO:7, the binding site sequence having at least 90% sequence identity (e.g., about 90, 91, 92, 93, 94, 95, 96, 70, 98, 99, or 100%) to SEQ ID NO:2, thereby generating an edit in the endogenous GRF transcription factor gene of the plant cell.

In some embodiments, the present invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO: 8, and comprising a miR396 binding site sequence at bp position 21 to 42 of SEQ ID NO:8, the binding site sequence having at least 90% sequence identity (e.g., about 90, 91, 92, 93, 94, 95, 96, 70, 98, 99, or 100%) to SEQ ID NO:3, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:4, and comprising a miR396 binding site sequence at bp position 21 to 42 of SEQ ID NO:4, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:5, and comprising a miR396 binding site sequence at bp position 21 to 42 of SEQ ID NO:5, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, the present invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:9, and comprising a miR396 binding site sequence at bp position 21 to 42 of SEQ ID NO:9, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:10, and comprising a miR396 binding site sequence at bp position 21 to 42 of SEQ ID NO:10, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, the present invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:11, and comprising a miR396 binding site sequence at bp position 21 to 42 of SEQ ID NO:11, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:12, and comprising a miR396 binding site sequence at bp position 21 to 42 of SEQ ID NO:12, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:13, and comprising a miR396 binding site sequence at bp position 21 to 42 of SEQ ID NO:13, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:14, and comprising a miR396 binding site sequence at bp position 21 to 42 of SEQ ID NO:14, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:15, and comprising a miR396 binding site sequence at bp position 21 to 42 of SEQ ID NO:15, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:16, and comprising a miR396 binding site sequence at bp position 21 to 42 of SEQ ID NO:16, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:17, and comprising a miR396 binding site sequence at bp position 21 to 42 of SEQ ID NO:17, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, the invention provides a method for editing a specific site in the genome of a plant cell, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NO:18, and comprising a miR396 binding site sequence at bp position 21 to 42 of SEQ ID NO:18, the binding site sequence having at least 90% sequence identity (e.g., about 90, 91, 92, 93, 94, 95, 96, 70, 98, 99, or 100%) to SEQ ID NO:1, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one of SEQ ID NOs:121-144 and comprising a miR396 binding site sequence at bp position 21 to 42 of any one of SEQ ID NOs: 121-144, the binding site sequence having at least 90% sequence identity (e.g., about 90, 91, 92, 93, 94, 95, 96, 70, 98, 99, or 100%) to SEQ ID NO: 145 or SEQ ID NO:146, thereby generating an edit in the endogenous GRF transcription factor gene of the plant cell.

In some embodiments, a method for editing a specific site in the genome of a plant cell is provided, the method comprising: cleaving, in a site specific manner, a target site within an endogenous GRF transcription factor gene in the plant cell, the endogenous GRF transcription factor gene comprising a sequence having about 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one of SEQ ID NOs:173-198 and comprising a miR396 binding site sequence at bp position 21 to 42 of any one of SEQ ID NOs: 173-198, the binding site sequence having at least 90% sequence identity (e.g., about 90, 91, 92, 93, 94, 95, 96, 70, 98, 99, or 100%) to SEQ ID NO:145 or SEQ ID NO:146, thereby generating an edit in the endogenous GRF transcription factor gene of the plant cell.

In some embodiments, the method may further comprise regenerating a plant from the plant cell comprising an edit in the endogenous GRF transcription factor gene to produce a plant comprising the edit in its endogenous GRF transcription factor gene. In some embodiments, the plant comprising the edit in its endogenous GRF transcription factor gene may have increased growth compared to a control plant that does not comprise the edit. In some embodiments, the edit results in a non-natural mutation.

In some embodiments, a method for making a plant is provided, the method comprising: (a) contacting a population of plant cells comprising an endogenous gene encoding a GRF transcription factor with an editing system comprising a nucleic acid binding domain that binds to a sequence having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of SEQ ID NOs: 1-3, 145, 146, or 199-202 or SEQ ID NOs:4-18, 121-144, or 173-198; (b) selecting a plant cell from said population comprising a mutation in at least one endogenous gene encoding a GRF transcription factor, wherein the mutation is a substitution of at least one nucleotide in the at least one endogenous gene, wherein the mutation reduces or eliminates the ability of miR396 to bind to a mRNA produced by the at least one endogenous gene encoding a GRF transcription factor comprising the mutation; and (c) growing the selected plant cell into a plant.

In some embodiments, the present invention provides a method for producing a plant or part thereof comprising at least one cell in which an endogenous GRF transcription factor gene is mutated, the method comprising contacting a target site in the GRF transcription factor gene in the plant or plant part with an editing system comprising a nucleic acid binding domain that binds to a target site in (or within) the GRF transcription factor gene having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one of SEQ ID NOs: 1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs: 19-33, 97-120, or 147-172, thereby producing a plant or part thereof comprising at least one cell having a mutation in the endogenous GRF transcription factor gene. In some embodiments, a mutated endogenous GRF transcription factor gene produces a mRNA that has reduced binding of its corresponding miR396 (e.g., the miR396 that naturally binds to the wild type GRF transcription factor mRNA).

In some embodiments, the present invention provides a method of producing a plant or part thereof that comprises a mutated endogenous GRF transcription factor gene that produces an mRNA having reduced miR396 binding, the method comprising contacting a target site in an endogenous GRF contacting a target site in an endogenous GRF transcription factor gene with an editing system comprising a nucleic acid binding domain that binds to a target site in the GRF transcription factor gene having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one of SEQ ID NOs:1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs: 19-33, 97-120, or 147-172, thereby producing a plant or part thereof comprising a mutated endogenous GRF transcription factor gene producing a mRNA having reduced miR396 binding. In some embodiments, the nucleotide sequence of any one of SEQ ID NOs:4-18, 121-144, or 173-198, or SEQ ID NOs: 19-33, 97-120, or 147-172 comprises a sequence having at least 90% sequence identity (e.g., about 90, 91, 92, 93, 94, 95, 96, 70, 98, 99, or 100%) to the nucleotide sequence of any one of SEQ ID NOs:1-3, 145, 146, or 199-202, the sequence having at least 90% sequence identity to the nucleotide sequence of any one of SEQ ID NOs:1-3, 145, 146, or 199-202 is modified.

In some embodiments, a plant comprising a mutated endogenous GRF transcription factor gene that produces a mRNA having reduced miR396 binding exhibits increased growth as compared to a control plant (e.g., a plant that does not comprise said modification or mutation in its endogenous GRF transcription factor gene or in which the plant's endogenous GRF transcription factor gene has not been contacted with the editing system). In some embodiments, a plant comprising a mutated endogenous GRF transcription factor gene that produces a mRNA having reduced miR396 binding exhibits at least one of the following phenotypes of increased meristem size, increased seed size, increased biomass, increased leaf size, increased root size, increased nitrogen use efficiency, increased disease resistance, increased height and/or increased internode length as compared to a control plant (e.g., a plant that does not comprise said modification or mutation in its endogenous GRF transcription factor gene or in which the plant's endogenous GRF transcription factor gene has not been contacted with the editing system).

In some embodiments, the present invention provides a method of producing a plant or part thereof having increased growth or an increased growth rate, the method comprising contacting a target site in an endogenous GRF transcription factor gene with an editing system comprising a nucleic acid binding domain that binds to a target site in the GRF transcription factor gene having at least 80% sequence identity (e.g., about 80, 81, 82, 83, 84, 85, 86, 87, 88, 89, 90, 91, 92, 93, 94, 95, 96, 70, 98, 99, or 100%) to the nucleotide sequence of any one SEQ ID NOs: 1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs: 19-33, 97-120, or 147-172, thereby producing a plant or part thereof comprising a mutated endogenous GRF transcription factor gene that produces a mRNA having reduced miR396 binding, thereby producing a plant or part thereof having increased growth or an increased growth rate. In some embodiments, the nucleotide sequence of any one of SEQ ID NOs:4-18, 121-144, or 173-198 or SEQ ID NOs:19-33, 97-120, or 147-172 comprises a sequence having at least 90% sequence identity (e.g., about 90, 91, 92, 93, 94, 95, 96, 70, 98, 99, or 100%) to the nucleotide sequence of any one of SEQ ID NOs: 1-3, 145, 146, or 199-202, the sequence having at least 90% sequence identity to the nucleotide sequence of any one of SEQ ID NOs:1-3, 145, 146, or 199-202 is modified.

An editing system useful with this invention can be any site-specific (sequence-specific) genome editing system now known or later developed, which system can introduce mutations in target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing a GRF transcription factor may comprise contacting a target nucleic acid (e.g., a nucleic acid encoding a GRF transcription factor) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid (see, e.g., FIG. 2). In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific DNA binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a GRF transcription factor may comprise contacting a target nucleic acid (e.g., a nucleic acid encoding a GRF transcription factor) with a sequence-specific DNA binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific DNA binding fusion protein to the target nucleic acid and the sequence-specific DNA binding fusion protein is capable of recruiting the deaminase fusion protein to the target nucleic acid via the peptide tag-affinity polypeptide interaction, thereby editing a locus within the target nucleic acid. In some embodiments, the sequence-specific DNA binding fusion protein may be fused to the affinity polypeptide that binds the peptide tag and the deaminase may be fuse to the peptide tag, thereby recruiting the deaminase to the sequence-specific DNA binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific DNA binding fusion proteins, deaminase fusion proteins and guides may be provided as ribonucleoproteins (RNPs).

In some embodiments, methods such as prime editing may be used to generate a mutation in an endogenous GRF transcription factor gene. In prime editing, RNA-dependent DNA polymerase (reverse transcriptase, RT) and reverse transcriptase templates (RT template) are used in combination with sequence specific DNA binding domains that confer the ability to recognize and bind the target in a sequence-specific manner, and which can also cause a nick of the PAM-containing strand within the target. The DNA binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantageous of the various methods of recruiting proteins for use in the editing to the target site, such methods including both non-covalent and covalent interactions between the proteins and nucleic acids used in the selected process of genome editing.

In some embodiments, the mutation or modification may be an insertion, a deletion and/or a point mutation. In some embodiments, a plant part may be a cell. In some embodiments, the plant or plant part thereof may be any plant or part thereof as described herein. In some embodiments, a plant useful with this invention may be corn, soy, canola, wheat, rice, cotton, sugarcane, sugar beet, barley, oats, alfalfa, sunflower, safflower, oil palm. sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, or a Brassica spp. In some embodiments, a plant comprising a mutated endogenous GRF transcription factor gene that produces a mRNA having reduced miR396 binding may comprise increased growth and/or an increased growth rate, optionally an increased growth and/or increased growth rate of its roots and/or fruit. In some embodiments, the plant may be corn and the fruit may be an ear, optionally wherein the corn plant comprises a short stature/semi-dwarf phenotype.

In some embodiments, a mutation that is introduced into an endogenous GRF transcription factor gene and resulting in the production of a mRNA having reduced miR396 binding may be a non-naturally occurring mutation. In some embodiments, a mutation that is introduced into an endogenous GRF transcription factor gene and resulting in the production of a mRNA having reduced miR396 binding may be a substitution, an insertion and/or a deletion. In some embodiments, a mutation that is introduced into an endogenous GRF transcription factor gene and resulting in the production of a mRNA having reduced miR396 binding may be a point mutation.

In some embodiments, a GRF transcription factor may include, but is not limited to, a GRF1, GRF2, GRF3, GRF4, GRF5, GRF6, GRF7, GRF8, GRF9, GRF10, GRF11, GRF12, GRF13, GRF14, GRF15, GRF16, GRF17, GRF18, GRF19 or a GRF20. In some embodiments, a GRF transcription factor may include, but is not limited to, a GRF6, GRF2, GRF1, GRF14, GRF15, GRF8, GRF4, GRF12, GRF10, GRF11, GRF5, GRF3, GRF9, GRF13, or a GRF7.

In some embodiments, the present invention further provides a method of producing/breeding a transgene-free base-edited plant, the method comprising: (a) crossing a plant of this invention comprising at least one mutation/modification in at least one endogenous GRF transcription factor gene that results in a mRNA having reduced binding of the corresponding miR396 with a transgene free plant, thereby introducing the at least one mutation/modification into the plant that is transgene-free; and (b) selecting a progeny plant that comprises the at least one single nucleotide substitution but is transgene-free, thereby producing a transgene free base-edited plant.

In some embodiments, a plant comprising a mutated endogenous GRF transcription factor gene that produces a mRNA having reduced miR396 binding may exhibit increased growth as compared to a control plant (e.g., a plant that does not comprise said modification or mutation in its endogenous GRF transcription factor gene or in which the plant's endogenous GRF transcription factor gene has not been contacted with the nuclease). In some embodiments, a plant comprising a mutated endogenous GRF transcription factor gene that produces a mRNA having reduced miR396 binding may exhibit at least one of the following phenotypes of increased meristem size, increased seed size, increased biomass, increased leaf size, increased root size, increased nitrogen use efficiency, increased disease resistance, increased height and/or increased internode length as compared to a control plant (e.g., a plant that does not comprise said modification or mutation in its endogenous GRF transcription factor gene or in which the plant's endogenous GRF transcription factor gene has not been contacted with the nuclease).

The present invention further provides a guide nucleic acid (e.g., gRNA) that binds to a target site in a GRF transcription factor gene, the target site comprising any one of the nucleotide sequences of SEQ ID NOs:4-18, 121-144, or 173-198, optionally wherein the GRF transcription factor may be a GRF1, GRF2, GRF3, GRF4, GRF5, GRF6, GRF7, GRF8, GRF9, GRF10, GRF11, GRF12, GRF13, GRF14, GRF15, GRF16, GRF17, GRF18, GRF19 or a GRF20. In some embodiments, a guide nucleic acid (e.g., gRNA) that binds to a target site in a GRF transcription factor gene is provide, the target site comprising any one of the nucleotide sequences of SEQ ID NOs:4-18, 121-144, or 173-198, optionally wherein the GRF transcription factor may be a GRF6, GRF2, GRF1, GRF14, GRF15, GRF8, GRF4, GRF12, GRF10, GRF11, GRF5, GRF3, GRF9, GRF13, or a GRF7.

In some embodiments, a guide nucleic acid of a gene editing system of this invention may comprise a spacer that is complementary to a fragment or portion of the nucleotide sequence of any one of SEQ ID NOs:4-18, 121-144, or 173-198, wherein the fragment or portion of any one of SEQ ID NOs:4-18, 121-144, or 173-198 may be from base pair position 5, 6, 7, 8, 9, 10, 11, 12, 13, 14, 15, 20, 21, 22, 23, 24, 25, 26, 27, 28, 29, 30, 31, 32, 33, 34, 35 to base pair position 36, 37, 38, 39, 40, 41, 42, 43, 44, 45, 46, 47, 48, 49, 50, 51, 52, 53, 54, 55, 56, 57, 58, 59, 60 of any one of SEQ ID NOs:4-18, 121-144, or 173-198. In some embodiments, a guide nucleic acid may comprise a spacer having complementary to consecutive nucleotides from base pair position 1 to base pair position 21 or 22, from base pair position 5 to base pair position 21, 22, 23, 24, 25, 26 or 27, from base pair position 10 to base pair position 25, 26, 27, 28, 29, 30, 31, or 32, from base pair position 15 to base pair position 30, 31, 32, 33, 34, 35, 36, or 37, from base pair position 20 to base pair position 35, 36, 37, 38, 39, 40, 41, or 42, from base pair position 21 to base pair position 37, 38, 39, 40, 41, 42, 43, or 44, from base pair position 25 to base pair position 40, 41, 42, 43, 44, 45, 46, 47, or 48, from base pair position 30 to base pair position 45, 46, 47, 48, 49, 50, 51, or 52, from base pair position 35 to base pair position 50, 51, 52, 53, 54, 55, 56, or 57, from base pair position 40 to base pair position 55, 56, 57, 58, 59, 60, 61, or 62, from base pair position 42 to base pair position 57, 58, 59, 60, 61, or 62, and the like, of any one of the nucleotide sequences of SEQ ID NOs:4-18, 121-144, or 173-198 (counted from the 5′ end). In some embodiments, a guide nucleic acid may comprise a spacer that is complementary to consecutive nucleotides from base pair position 20 to base pair position 42 of any one of the nucleotide sequences of SEQ ID NOs:4-18, 121-144, or 173-198 (counted from the 5′ end). In some embodiments, the guide nucleic acid may comprise a spacer having the nucleotide sequence of any one of SEQ ID NOs:48-71.

The present invention further provides an editing system comprising a guide nucleic acid of the invention and a CRISPR-Cas effector protein that associates with the guide nucleic acid. In some embodiments, an editing system of the invention may further comprise tracr nucleic acid that associates with the guide nucleic acid and a CRISPR-Cas effector protein, optionally wherein the tracr nucleic acid and the guide nucleic acid are covalently linked.

The present invention further provides an editing system comprising a CRISPR-Cas effector protein in association with a guide nucleic acid, wherein the guide nucleic acid comprises a spacer sequence that binds to a GRF transcription factor gene. In some embodiments, the GRF transcription factor gene may be a GRF1 gene, GRF2 gene, GRF3 gene, GRF4 gene, GRF5 gene, GRF6 gene, GRF7 gene, GRF8 gene, GRF9 gene, GRF10 gene, GRF11 gene, GRF12 gene, GRF13 gene, GRF14 gene, GRF15 gene, GRF16 gene, GRF17 gene, GRF18 gene, GRF19 gene, or a GRF20 gene. In some embodiments, the GRF transcription factor gene may be a GRF6 gene, GRF2 gene, GRF1 gene, GRF14 gene, GRF15 gene, GRF8 gene, GRF4 gene, GRF12 gene, GRF10 gene, GRF11 gene, GRF5 gene, GRF3 gene, GRF9 gene, GRF13 gene, or a GRF7 gene. In some embodiments, the GRF transcription factor gene of the gene editing system may comprise the nucleotide sequence of any one of SEQ ID NOs:1-3, 145, 146, or 199-202.

In some embodiments, a guide nucleic acid of the gene editing system may comprise a spacer sequence having the nucleotide sequence of any one of SEQ ID NOs:48-71.

The present invention further provides a complex comprising a CRISPR-Cas effector protein comprising a cleavage domain and a guide nucleic acid (e.g., gRNA), wherein the guide nucleic acid binds to a target site in a GRF transcription factor gene having the nucleotide sequence of any one SEQ ID NOs:4-18, 121-144, or 173-198, wherein the nuclease cleaves the target strand.

Further provided herein are expression cassettes comprising a (a) polynucleotide encoding CRISPR-Cas effector protein comprising a cleavage domain and (b) a guide nucleic acid that binds to a target site in a GRF transcription factor gene, wherein the guide nucleic acid comprises a spacer sequence that is complementary to and binds to a sequence having at least 80% sequence identity to at least a portion of any one of the nucleotide sequences of SEQ ID NOs:1-3, 145, 146, or 199-202 or a sequence having at least 80% sequence identity to at least a portion of any one of the nucleotide sequences of SEQ ID NOs:4-18, 121-144, or 173-198, as described herein. In some embodiments, the at least a portion may comprise at least one nucleotide or at least two (e.g., 2, 3, 4, 5, 6, 7, 8, 9, 10, 11, 12, 13, 14, 15, 16, 17, 18, 19, 20, 21, or 22) consecutive nucleotides of any one of the nucleotide sequences of SEQ ID NOs:1-3, 145, 146, or 199-202.

In some embodiments, the at least a portion may be a portion of the nucleotide sequence of any one of SEQ ID NOs: 1-3, 145, 146, or 199-202 and/or any one of SEQ ID NOs:4-18, 121-144, or 173-198, wherein the portion of any one of SEQ ID NOs:1-3, 145, 146, or 199-202 and/or any one of SEQ ID NOs:4-18, 121-144, or 173-198 may be from base pair position 5, 6, 7, 8, 9, 10, 11, 12, 13, 14, 15, 20, 21, 22, 23, 24, 25, 26, 27, 28, 29, 30, 31, 32, 33, 34, 35 to base pair position 36, 37, 38, 39, 40, 41, 42, 43, 44, 45, 46, 47, 48, 49, 50, 51, 52, 53, 54, 55, 56, 57, 58, 59, 60 of any one of any one of SEQ ID NOs:1-3, 145, 146, or 199-202 and/or any one of SEQ ID NOs:4-18, 121-144, or 173-198. In some embodiments, the at least a portion may comprise consecutive nucleotides from base pair position 1 to base pair position 21 or 22, from base pair position 5 to base pair position 21, 22, 23, 24, 25, 26 or 27, from base pair position 10 to base pair position 25, 26, 27, 28, 29, 30, 31, or 32, from base pair position 15 to base pair position 30, 31, 32, 33, 34, 35, 36, or 37, from base pair position 20 to base pair position 35, 36, 37, 38, 39, 40, 41, or 42, from base pair position 21 to base pair position 37, 38, 39, 40, 41, 42, 43, or 44, from base pair position 25 to base pair position 40, 41, 42, 43, 44, 45, 46, 47, or 48, from base pair position 30 to base pair position 45, 46, 47, 48, 49, 50, 51, or 52, from base pair position 35 to base pair position 50, 51, 52, 53, 54, 55, 56, or 57, from base pair position 40 to base pair position 55, 56, 57, 58, 59, 60, 61, or 62, from base pair position 42 to base pair position 57, 58, 59, 60, 61, or 62, and the like, of any one of the nucleotide sequences of any one of SEQ ID NOs:1-3, 145, 146, or 199-202 and/or any one of SEQ ID NOs:4-18, 121-144, or 173-198 (counted from the 5′ end). In some embodiments, the at least a portion may comprise consecutive nucleotides from base pair position 20 to base pair position 42 of any one of the nucleotide sequences of any one of SEQ ID NOs: 1-3, 145, 146, or 199-202 and/or any one of SEQ ID NOs:4-18, 121-144, or 173-198 (counted from the 5′ end). In some embodiments, a portion of any one of SEQ ID NOs: 1-3, 145, 146, or 199-202 and/or any one of SEQ ID NOs:4-18, 121-144, or 173-198 may be at least about 1, 2, 3, 4, 5, 6, 7, 8, 9, 10, 11, 12, 13, 14, 15, 16, 17, 18, 19, 20, 21, 22, 23, 24, or 25 or more consecutive nucleotides of any one of SEQ ID NOs: 1-3, 145, 146, or 199-202 and/or any one of SEQ ID NOs:4-18, 121-144, or 173-198.

Also provided herein is a nucleic acid encoding GRF transcription factor that produces a mRNA having a mutated miR396 binding site, wherein the mutated miR396 binding site comprises a mutation that results in increased levels of the mRNA, and optionally, disrupts (reduced or eliminates) miR396 binding to the mRNA. In some embodiments, the mutation may eliminate the binding of the corresponding miR 396 (e.g., zero detectable binding). In some embodiments, the mutation may reduce the ability of the corresponding miR396 to bind to the GRF transcription factor mRNA by at least about 75% (e.g., about 75, 76, 77, 78, 79, 80, 81, 82, 83, 84, 85, 86, 87, 88, 89, 90, 91, 92, 93, 94, 95, 96, 97, 98, 99% or more, or any range or value therein, of a reduction in miR396 binding, or any range or value therein). In some embodiments, the GRF transcription factor may be a GRF1, GRF2, GRF3, GRF4, GRF5, GRF6, GRF7, GRF8, GRF9, GRF10, GRF11, GRF12, GRF13, GRF14, GRF15, GRF16, GRF17, GRF18, GRF19 or a GRF20. In some embodiments, the GRF transcription factor may be a GRF6, GRF2, GRF1, GRF14, GRF15, GRF8, GRF4, GRF12, GRF10, GRF11, GRF5, GRF3, GRF9, GRF13, or a GRF7. In some embodiments, a plant or part thereof is provided that comprises a nucleic acid of the invention encoding a GRF transcription factor that produces a mRNA having a mutated miR396 binding site, wherein the mutated miR396 binding site comprises a mutation that results in increased levels of the mRNA, and optionally, disrupts miR396 binding. In some embodiments the plant is a corn plant, optionally wherein the corn plant comprises a short stature/semi-dwarf phenotype. In some embodiments, the plant is a wheat plant or part thereof, optionally wherein the nucleic acid encoding a GRF transcription factor that produces a mRNA having a mutated miR396 binding site may be comprised in the A genome, the B genome, the D genome or in any combination thereof. In some embodiments, the plant or part thereof that comprises a nucleic acid of the invention encoding a GRF transcription factor that produces a mRNA having a mutated miR396 binding site may comprise increased growth or an increased rate of growth. In some embodiments, the plant or part thereof that comprises a nucleic acid of the invention encoding a GRF transcription factor that produces a mRNA having a mutated miR396 binding site may exhibit at least one of the following phenotypes of increased meristem size, increased seed size, increased biomass, increased leaf size, increased root size, increased nitrogen use efficiency, increased disease resistance, increased height and/or increased internode length.

In some embodiments, the present invention further provides a guide nucleic acid that binds to a target nucleic acid in a GRF transcription factor in a corn plant, wherein the target nucleic acid is located in: (a) a chromosome interval defined by and including base pair (bp) position 211773221 to base pair position 211774543 on chromosome 5, optionally a chromosome interval defined by and including base pair (bp) position 211773897 to base pair position 211773957; (b) a chromosome interval defined by and including base pair (bp) position 200344009 to base pair position 200348833 on chromosome 5, optionally a chromosome interval defined by and including base pair (bp) position 200346611 to base pair position 200346671; (c) a chromosome interval defined by and including base pair (bp) position 13817620 to base pair position 13822440 on chromosome 5, optionally a chromosome interval defined by and including base pair (bp) position 138197933 to base pair position 13819853; (d) a chromosome interval defined by and including base pair (bp) position 177151736 to base pair position 177154443 on chromosome 4, optionally a chromosome interval defined by and including base pair (bp) position 177152804 to base pair position 177152864; (c) a chromosome interval defined by and including base pair (bp) position 257245490 to base pair position 257249295 on chromosome 1, optionally a chromosome interval defined by and including base pair (bp) position 257247225 to base pair position 257247285; (f) a chromosome interval defined by and including base pair (bp) position 25722859 to base pair position 25725873 on chromosome 9, optionally a chromosome interval defined by and including base pair (bp) position 25723770 to base pair position 25723830; (g) a chromosome interval defined by and including base pair (bp) position 60351912 to base pair position 60356302 on chromosome 6, optionally a chromosome interval defined by and including base pair (bp) position 60354489 to base pair position 60354549; (h) a chromosome interval defined by and including base pair (bp) position 196192921 to base pair position 196194872 on chromosome 5, optionally a chromosome interval defined by and including base pair (bp) position 196194175 to base pair position 196194235; (i) a chromosome interval defined by and including base pair (bp) position 140393394 to base pair position 140398705 on chromosome 10, optionally a chromosome interval defined by and including base pair (bp) position 140396272 to base pair position 140396332; (j) a chromosome interval defined by and including base pair (bp) position 155831622 to base pair position 155833517 on chromosome 4, optionally a chromosome interval defined by and including base pair (bp) position 155832749 to base pair position 155832809; (k) a chromosome interval defined by and including base pair (bp) position 12201898 to base pair position 12208052 on chromosome 2, optionally a chromosome interval defined by and including base pair (bp) position 12204599 to base pair position 12204659; (1) a chromosome interval defined by and including base pair (bp) position 9818327 to base pair position 9820186 on chromosome 9, optionally a chromosome interval defined by and including base pair (bp) position 9819224 to base pair position 9819284; (m) a chromosome interval defined by and including base pair (bp) position 108477686 to base pair position 108480099 on chromosome 6, optionally a chromosome interval defined by and including base pair (bp) position 108478747 to base pair position 108478807; (n) a chromosome interval defined by and including base pair (bp) position 225827712 to base pair position 225832487 on chromosome 2, optionally a chromosome interval defined by and including base pair (bp) position 225830099 to base pair position 225830159; (o) a chromosome interval defined by and including base pair (bp) position 272414778 to base pair position 272420567 on chromosome 1; (p) a chromosome interval defined by and including base pair (bp) position 199377455 to base pair position 199381799 on chromosome 2; (q) a chromosome interval defined by and including base pair (bp) position 145016279 to base pair position 145019899 on chromosome 7; and/or (r) a chromosome interval defined by and including base pair (bp) position 8708791 to base pair position 8711617 on chromosome 5, wherein the mutation results in increased levels of the mRNA, and optionally, disrupts the binding of miR396 to the GRF transcription factor, wherein the chromosomal intervals of (a) to (r) correspond to the reference corn genome of B73 (see e.g., Table 1).

In some embodiments, a sequence-specific nucleic acid binding domain (DNA binding domains) of an editing system useful with this invention can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein.

In some embodiments, a sequence-specific DNA binding domain may be a CRISPR-Cas effector protein, optionally wherein the CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csy1, Csy2, Csy3, Csel, Csc2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g, Cas9 nickase, Cas12a nickase.

A CRISPR Cas9 effector protein or CRISPR Cas9 effector domain useful with this invention may be any known or later identified Cas9 nuclease (see, e.g., SEQ ID NOs:234-250). In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. FIGS. 4A-4B show example base edits using Cas9.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (Sec, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (Sec, e.g., Hou et al, PNAS 2013, 1-6). In the aforementioned embodiments, N can be any nucleotide residue, e.g., any of A, G, Cor T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease (see, e.g., SEQ ID NOs:214-230, SEQ ID NOs:231-233). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

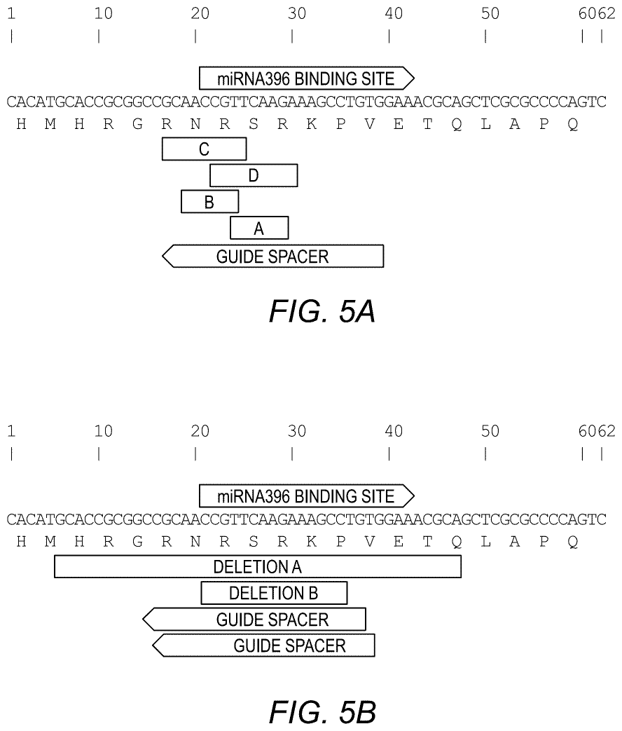

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity. FIG. 5A-5B show example base edits using Cas12a (Cpf1).

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally-occurring cytosine deaminase, including but not limited to a primate (e.g., a human, monkey, chimpanzee, gorilla), a dog, a cow, a rat or a mouse. Thus, in some embodiments, an cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:206, SEQ ID NO:207 or SEQ ID NO:208). In some embodiments, the cytosine deaminase may be an APOBEC1 deaminase having the amino acid sequence of SEQ ID NO:77. In some embodiments, the cytosine deaminase may be an APOBEC3A deaminase having the amino acid sequence of SEQ ID NO:78. In some embodiments, the cytosine deaminase may be an CDA1 deaminase, optionally a CDA1 having the amino acid sequence of SEQ ID NO:79. In some embodiments, the cytosine deaminase may be a FERNY deaminase, optionally a FERNY having the amino acid sequence of SEQ ID NO:80. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:77, SEQ ID NO:78, SEQ ID NO:79 or SEQ ID NO:80 (e.g., at least 80%, at least 85%, at least 90%, at least 92%, at least 95%, at least 96%, at least 97%, at least 98%, at least 99%, or at least 99.5% identical to the amino acid sequence of SEQ ID NO:77, SEQ ID NO:78, SEQ ID NO:79 or SEQ ID NO:80, SEQ ID NO:206, SEQ ID NO:207 or SEQ ID NO:208). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:81 or a polypeptide having about 70% to about 99.5% identity to the amino acid sequence of SEQ ID NO:81 (e.g., at least 80%, at least 85%, at least 90%, at least 92%, at least 95%, at least 96%, at least 97%, at least 98%, at least 99%, or at least 99.5% identical to the amino acid sequence of SEQ ID NO:81). For example, in some embodiments, a UGI domain may comprise a fragment of the amino acid sequence of SEQ ID NO:81 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:81. In some embodiments, a UGI domain may be a variant of a known UGI (e.g., SEQ ID NO:81) having about 70% to about 99.5% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally-occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:72. In some embodiments, a mutated/evolved E. coli TadA* comprises the amino acid sequence of SEQ ID NOs:73-76 or 209-213 (e.g., SEQ ID NOs:73, 74, 75, 76, 209, 210, 211, 212 or 213). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Csel, Csc2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2cl CRISPR Cas system, or a fragment thereof; a repeat of a CRISPR-Cas system of, for example, C2c3, Cas12a (also referred to as Cpf1), Cas12b, Cas12c, Cas12d, Cas12c, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csy1, Csy2, Csy3, Csel, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csfl, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPR finder offered through CRISPRdb (see, Grissa et al. Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g, protospacer) (e.g., consecutive nucleotides of any one of SEQ ID NOs:1-18) (e.g., SEQ ID NOs:48-71). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

| | RNA Spacer |
|---|---|
| (SEQ ID NO: 203) | |
| | 5′-NNNNNNNNNNNNNNNNNNN-3′ |
| | \|\|\|\|\|\|\|\|\|\|\|\|\|\|\|\|\|\|\| |
| | Target strand |
| (SEQ ID NO: 204) | |
| | 3′AAANNNNNNNNNNNNNNNNNNN-5′ |
| | \|\|\|\| |
| | Non-target strand |
| (SEQ ID NO: 205) | |
| | 5′TTTNNNNNNNNNNNNNNNNNNN-3′ |

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific DNA binding domains, CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases).

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together; e.g. dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

In some embodiments, the invention provides cells comprising one or more polynucleotides, guide nucleic acids, nucleic acid constructs, expression cassettes or vectors of the invention.

In some embodiments, a method of editing an endogenous GRF transcription factor gene in a plant or plant part is provided, the method comprising contacting a target site in the GRF transcription factor gene in the plant or plant part with a cytosine base editing system comprising a cytosine deaminase and a nucleic acid binding domain that binds to a target site in the GRF transcription factor gene having at least 80% sequence identity to the nucleotide sequence of any one of SEQ ID NOs:1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs: 19-33, 97-120, or 147-172, wherein the cytosine deaminase generates at least one C to T conversion in the nucleotide sequence of any one of SEQ ID NOs:1-3, 145, 146, or 199-202, thereby producing a plant or part thereof comprising at least one cell having a mutation in the endogenous GRF transcription factor gene.

In some embodiments, a method of editing an endogenous GRF transcription factor gene in a plant or plant part is provided, the method comprising contacting a target site in the GRF transcription factor gene in the plant or plant part with an adenine base editing system comprising an adenine deaminase and a nucleic acid binding domain that binds to a target site in the GRF transcription factor gene having at least 80% sequence identity to the nucleotide sequence of any one of SEQ ID NOs:1-3, 145, 146, or 199-202, having at least 80% sequence identity to the nucleotide sequence of any one of SEQ ID NOs:4-18, 121-144, or 173-198, or having at least 80% sequence identity to the nucleotide sequence of any one of SEQ ID NOs: 19-33, 97-120, or 147-172, wherein the cytosine deaminase generates at least one A to G conversion in the nucleotide sequence of any one of SEQ ID NOs:1-3, 145, 146, or 199-202, thereby producing a plant or part thereof comprising at least one cell having a mutation in the endogenous GRF transcription factor gene.

In some embodiments, a method of detecting a mutant GRF (a mutation in an endogenous GRF transcription factor gene) is provide, the method comprising detecting in the genome of a plant a base substitution in at least one of positions 11, 19, 21 and/or 22 of the nucleotide sequence of any one of SEQ ID NOs:1-3, 145, 146, or 199-202. In some embodiments, the substitution that is detected at position 11 of the nucleotide sequence of any one of SEQ ID NOs:1-3, 145, 146, or 199-202 may be from G>A. In some embodiments, the substitution that is detected at position 19 of the nucleotide sequence of any one of SEQ ID NOs:1-3, 145, 146, or 199-202 may be from G>A or G>C. In some embodiments, the substitution that is detected at position that is detected at position 21 of the nucleotide sequence of any one of SEQ ID NOs:1-3, 145, 146, or 199-202 may be from G>A or G>C. In some embodiments, the substitution that is detected at position 22 of the nucleotide sequence of any one of SEQ ID NOs: 1-3, 145, 146, or 199-202 may be from G>A or G>C.

In some embodiments, the present invention provides a method of detecting a mutation in an endogenous GRF gene, comprising detecting in the genome of a plant one or more of any one of the nucleotide sequences of SEQ ID NOs:34-41.

In some embodiments, the present invention provides a method of producing a plant comprising a mutation in an endogenous GRF transcription factor gene and at least one polynucleotide of interest, the method comprising crossing a plant of the invention comprising at least one mutation in an endogenous GRF transcription factor gene (a first plant) with a second plant that comprises the at least one polynucleotide of interest to produce progeny plants; and selecting progeny plants comprising at least one mutation in the GRF transcription factor gene and the at least one polynucleotide of interest, thereby producing the plant comprising a mutation in an endogenous GRF transcription factor gene and at least one polynucleotide of interest.

The present invention further provides a method of producing a plant comprising a mutation in an endogenous GRF transcription factor gene and at least one polynucleotide of interest, the method comprising introducing at least one polynucleotide of interest into a plant of the present invention comprising at least one mutation in a GRF transcription factor gene, thereby producing a plant comprising at least one mutation in a GRF transcription factor gene and at least one polynucleotide of interest.

In some embodiments, the present invention provides a method of producing a plant comprising a mutation in an endogenous GRF transcription factor gene and at least one polynucleotide of interest, the method comprising introducing at least one polynucleotide of interest into a plant of the invention comprising at least one mutation in an endogenous GRF transcription factor gene, thereby producing a plant comprising at least one mutation in a GRF transcription factor gene and at least one polynucleotide of interest.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, disease resistance, increased yield, increased nutrient use efficiency or abiotic stress resistance.

In some embodiments, a method of producing a plant comprising a mutation in an endogenous GRF transcription factor gene and having a dwarf or short stature phenotype is provided, the method comprising crossing a plant of the invention (a first plant) having at least one mutation in an endogenous GRF transcription factor gene with a second plant that comprises the dwarf or short stature phenotype to produce progeny plants; and selecting progeny plants comprising the at least one mutation in the GRF transcription factor gene and the dwarf or short stature phenotype, thereby producing the plant having a dwarf or short stature and comprising at least one mutation in an endogenous GRF transcription factor gene.

The present invention further provides a method of controlling weeds in a container (e.g., pot, or seed tray and the like), a growth chamber, a greenhouse, a field (e.g., a cultivated field), a recreational area, a lawn, and/or a roadside, comprising applying an herbicide to one or more (a plurality) plants of the present invention growing in the container, growth chamber, field or greenhouse, thereby controlling the weeds in the container, growth chamber, greenhouse, field, recreational area, a lawn, and/or a roadside in which the one or more plants are growing.

In some embodiments, a method of reducing insect predation on a plant (or a plurality of plants) is provided, comprising applying an insecticide to one or more (a plurality) plants of the present invention, thereby reducing the insect predation on the one or more (a plurality) plants. In some embodiments the one or more plants may be growing in a container, a growth chamber, a field, a recreational area (e.g., playing field, golf course), a lawn, roadside, or a greenhouse.

In some embodiments, the present invention provides a method of reducing fungal disease on a plant, comprising applying a fungicide to one or more (a plurality) plants of the present invention, thereby reducing fungal disease on the on the one or more (a plurality) plants. In some embodiments the one or more plants may be growing in a container, a growth chamber, a field, a recreational area (e.g., playing field, golf course), a lawn, a roadside, or a greenhouse.

The nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

A target nucleic acid of any plant or plant part (or groupings of plants, for example, into a genus or higher order classification) may be modified (e.g., mutated, e.g., base edited, cleaved, nicked, etc.) using the polypeptides, polynucleotides, ribonucleoproteins (RNPs), nucleic acid constructs, expression cassettes, and/or vectors of the invention including an angiosperm, a gymnosperm, a monocot, a dicot, a C3, C4, CAM plant, a bryophyte, a fern and/or fern ally, a microalgae, and/or a macroalgae. A plant and/or plant part that may be modified as described herein may be a plant and/or plant part of any plant species/variety/cultivar. In some embodiments, a plant that may be modified as described herein is a monocot. In some embodiments, a plant that may be modified as described herein is a dicot.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, cars, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, collenchyma cells, sclerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

Non-limiting examples of plants that may be modified as described herein may include, but are not limited to, turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, or sunflower.

In some embodiments, a plant that may be modified as described herein may include, but is not limited to, corn, soy, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, or a Brassica spp (e.g., B. napus, B. oleraceae, B. rapa, B. juncea, and/or B. nigra.

In some embodiments, a plant that may be modified as described herein is corn (i.e., maize, Zea mays, optionally wherein the corn plant comprises a short stature/semi-dwarf phenotype.

In some embodiments, a plant that may be modified as described herein is wheat (e.g., Triticum aestivum, T. durum, and/or T. compactum). In some embodiments, a wheat plant may comprise at least one non-natural mutation in an endogenous Growth Regulating Factor (GRF) transcription factor in its A genome, in its B genome, and/or in its D genome.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

Modification of Endogenous Maize GRF Transcription Factor Family with Base Editing

This example demonstrates the use of DNA base-editing to modify maize GRF transcription factors resulting in an increase in plant height and car length.

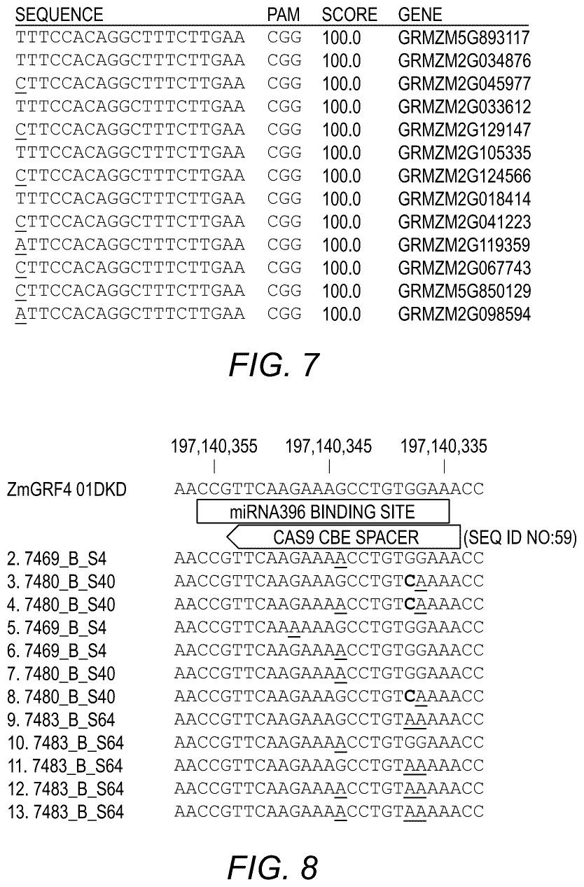

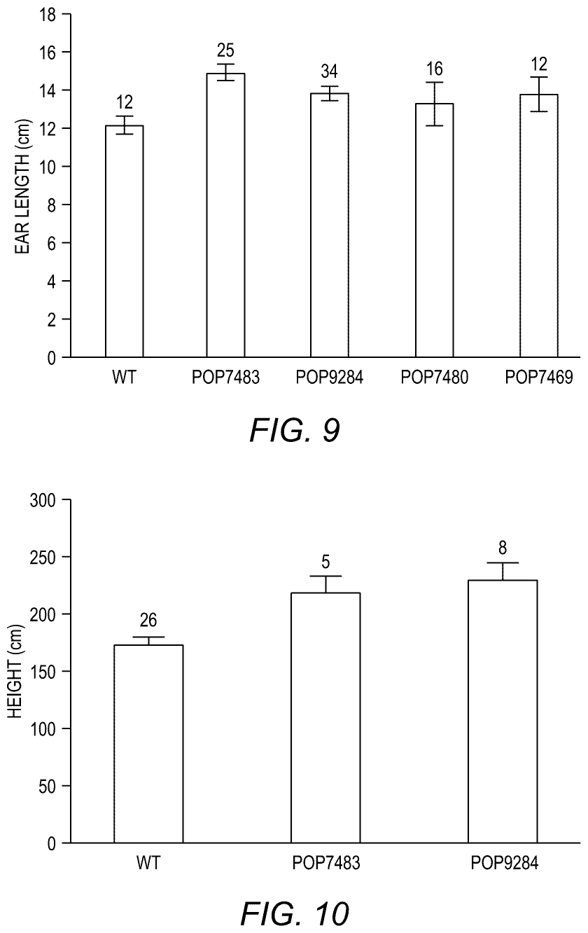

The spacer sequence comprising SEQ ID NO:58 was selected for use in a Cas9 base editing approach targeting the conserved miR396 region of several maize GRF transcription factors. GRF6 and four other GRF loci were targeted with 100% match to the spacer sequence, FIG. 7. A vector encoding the spacer as well as a mutated Cas9 effector fused to a cytosine deaminase were introduced into maize dry-excised embryos (DEEs) using Agrobacterium. After transformation, we assayed regenerating plants (E0 generation) for edits at the GRF6 locus with standard next-gen-sequencing (NGS) methods and identified several families with base-edit mutations in the miR396 targets site, FIG. 8. We grew four different populations for trait testing in the next generation, three derived from selfed E0 plants that showed editing at GRF6 (Population 7483, 7480, 7469), and a fourth with seed derived from a backcross of plant 7469 to the transformation germplasm (Population 9284). These four populations were grown in controlled environment and transgene-free plants were selfed and subjected to trait testing. Our results show the progeny of E0 base-edited plants have increased car length FIG. 9, and plant height FIG. 10. These results show that base editing within the miRNA396 target site of one of these GRF transcription factors results in increased maize plant growth.

### Example 2

Increasing the mRNA Level of Maize GRF6 Transcription Factor with Cpf1 In-Frame Deletion Gene Editing.

This example demonstrates the modification of an endogenous maize GRF6 transcription factor, GRMZM2G034876 (GRF6) to disrupt miRNA396 action and increase GRF6 mRNA levels using a Cpf1 cutting nuclease.

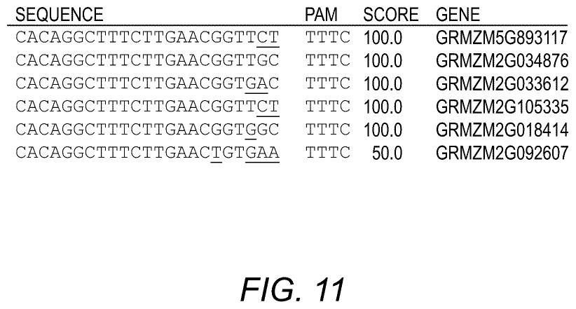

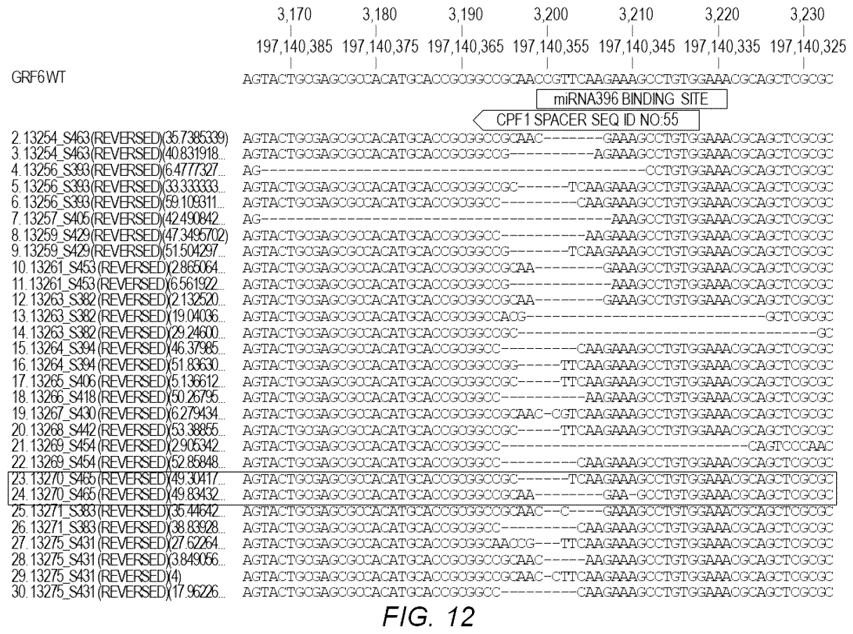

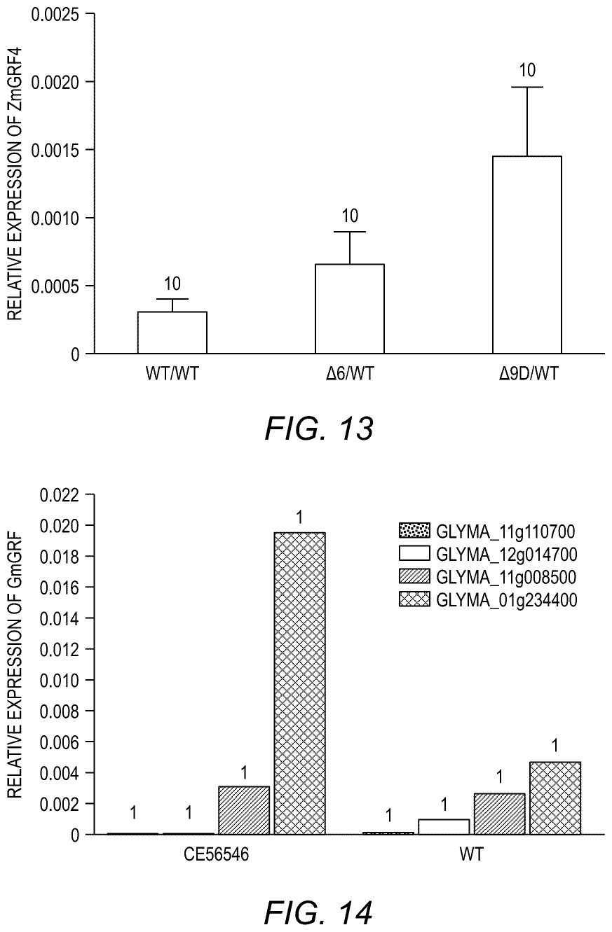

A spacer sequence comprising SEQ ID NO:55 with high specificity to the GRF6 target locus was designed. In this case only GRF6 was targeted with 100% specificity (FIG. 11). A vector encoding the spacer and a Cpf1-based nuclease were introduced into maize dry-excised embryos (DEEs) using Agrobacterium. After transformation, we assayed regenerating plants (E0 generation) for edits at the GRF6 locus with standard NGS methods and identified several families with in-frame deletions in the miR396 targets site, FIG. 12. Plant 13270 had ˜50% reads for a 6 bp in-frame deletion and ˜50% reads 9 bp in-frame deletion and was selected for further analysis. Plant 13270 was selfed and the progeny grown another generation (E1). In E1, a transgene-free plant containing both 6 bp and 9 bp deletions was backcrossed to the transformation germplasm (01DKD2). The progeny from this cross were grown, assayed for edits at GRF6, and subjected to trait testing. The GRF6 mRNA levels in leaf tissue of heterozygous 6 bp deletion, heterozygous 9 bp deletion, and a wildtype control plants were compared using standard quantitative PCR (qPCR) methods and we observed an increase in GRF6 mRNA levels in both heterozygous edit populations, FIG. 13. This example shows that in-frame deletion within the maize GRF6 miRNA396 target site using gene editing can increase GRF6 mRNA levels, even when the edit is in the heterozygous state. No statistical differences in plant phenotypes were observed, including, for example, plant height, ear length and 100 kernel weight. These results suggest it is one of the other GRFs and not GRF6 that explain the increased plant height and ear length resulting from the base edits in Example 1.

### Example 3

Increasing the mRNA Level of Soy GRF Transcription Factor with Cpf1 In-Frame Deletion Gene Editing.

This example demonstrates the modification of an endogenous soy GRF transcription factor (GLYMA_01 g234400) to disrupt miRNA396 action and increase GRF mRNA levels. A spacer simultaneously targeting four different GRF transcription factors (GLYMA_01 g234400 SEQ ID NO:97, GLYMA_11 g008500 SEQ ID NO:110, GLYMA_12 g014700 SEQ ID NO:108, GLYMA_11 g110700 SEQ ID NO:112) was designed (TGATTCCACAGGCTTTCTTGAAC (SEQ ID NO:251)). A vector expressing the spacer and a Cpf1-based nuclease was transformed into Soy dry-excised embryos (DEEs) using Agrobacterium. Regenerating plants were assayed for deletions at all four target loci using standard NGS methods. A plant (CE56546) was identified with a 6 bp in-frame deletion in the miR396 target site in locus GLYMA_01 g234400 at high % reads. Leaf tissue was sampled from this plant and the mRNA level of all four GRF loci were compared to wildtype controls using standard qPCR methods (FIG. 14). Only GLYMA_01 g234400 showed an increase in mRNA levels consistent with the high % editing of this locus. The other GRFs assayed showed relatively low % edit and no increase in mRNA levels. These results show that in-frame deletions within the miR396 target of Soy GRFs can also increase mRNA levels in this species.

TABLE 1
Maize GRF transcription factor gene names as provided in the V1-V4 B73 corn strain gene models along with corresponding GRF
name as use herein. Also provided are the chromosome positions of the GRF genes and miRNA binding sites (based on B73 RefGen v3)

| | | | | | | | v3_Binding |
|---|---|---|---|---|---|---|---|
| | | | | GRF | Chromo- | | Site Start |
| v1_gene_model | v2_gene_model | v3_gene_model | v4_gene_model | Name | some | v3_Coordinates | Position |
| GRMZM2G178261 | GRMZM2G178261 | GRMZM2G178261 | Zm00001d033876 | GRF1 | 1 | 1:272414778 . . . 272420567 | |
| GRMZM2G119359 | GRMZM2G119359 | GRMZM2G119359 | Zm00001d045533 | GRF2 | 9 | 9:25722859 . . . 25725873 | 25723800 |
| GRMZM2G048993 | GRMZM5G893117 | GRMZM5G893117 | Zm00001d018260 | GRF3 | 5 | 5:211773221 . . . 211774543 | 211773927 |
| GRMZM2G018414 | GRMZM2G018414 | GRMZM2G018414 | Zm00001d033396 | GRF4 | 1 | 1:257245490 . . . 257249295 | 257247255 |
| GRMZM2G129147 | GRMZM2G129147 | GRMZM2G129147 | Zm00001d026240 | GRF5 | 10 | 10:140393394 . . . 140398705 | 140396302 |
| GRMZM2G034876 | GRMZM2G034876 | GRMZM2G034876 | Zm00001d017742 | GRF6 | 5 | 5:200344009 . . . 200348833 | 200346641 |
| GRMZM2G098594 | GRMZM2G098594 | GRMZM2G098594 | Zm00001d035965 | GRF7 | 6 | 6:60351912 . . . 60356302 | 60354519 |
| GRMZM2G041223 | GRMZM2G041223 | GRMZM2G041223 | Zm00001d002429 | GRF8 | 2 | 2:12201898 . . . 12208052 | 12204629 |
| GRMZM2G124566 | GRMZM2G124566 | GRMZM2G124566 | Zm00001d051456 | GRF9 | 4 | 4:155831622 . . . 155833517 | 155832779 |
| GRMZM2G105335 | GRMZM2G105335 | GRMZM2G105335 | Zm00001d052112 | GRF10 | 4 | 4:177151736 . . . 177154443 | 177152834 |
| | GRMZM5G850129 | GRMZM5G850129 | Zm00001d037117 | GRF11 | 6 | 6:108477686 . . . 108480099 | 108478777 |
| GRMZM2G067743 | GRMZM2G067743 | GRMZM2G067743 | Zm00001d000238 | GRF12 | 9 | 9:9818327 . . . 9820186 | 9819254 |
| GRMZM2G033612 | GRMZM2G033612 | GRMZM2G033612 | Zm00001d013555 | GRF13 | 5 | 5:13817620 . . . 13822440 | 13819823 |
| | GRMZM5G853392 | GRMZM5G853392 | Zm00001d013346 | GRF14 | 5 | 5:8708791 . . . 8711617 | |
| GRMZM2G099862 | GRMZM2G099862 | GRMZM2G099862 | Zm00001d007465 | GRF15 | 2 | 2:225827712 . . . 225832487 | 225830129 |

The foregoing is illustrative of the present invention and is not to be construed as limiting thereof. The invention is defined by the following claims, with equivalents of the claims to be included therein.

What is claimed is:

1. A corn plant or plant part thereof comprising a mutated gene that comprises a nucleic acid sequence of any one of SEQ ID NOs:34-41 or 276-303, and
wherein the corn plant or plant part thereof has increased levels of mRNA compared to a control corn plant or plant part thereof that is devoid of the mutated gene.

2. The corn plant or plant part thereof of claim 1, wherein the corn plant comprises a short stature/semi-dwarf phenotype.

3. The corn plant or plant part thereof of claim 1, wherein at least one non-natural mutation in an endogenous gene encoding a Growth Regulating Factor (GRF) transcription factor results in the mutated gene.

4. The corn plant or plant part thereof of claim 3, wherein the endogenous GRF transcription factor gene comprises a sequence having at least 90% sequence identity to the nucleotide sequence of SEQ ID NO:304.

5. The corn plant or plant part thereof of claim 3, wherein the at least one non-natural mutation is located in a region of the endogenous GRF transcription factor gene, the region comprising the sequence of SEQ ID NO:275 and the at least one non-natural mutation resulting in the nucleic acid sequence of any one of SEQ ID NOs:276-303.

6. The corn plant or plant part thereof of claim 3, wherein the at least one non-natural mutation is located in a region of the endogenous GRF transcription factor gene, the region comprising the sequence of SEQ ID NO:254 and the at least one non-natural mutation resulting in the nucleic acid sequence of any one of SEQ ID NOs:34-41.

7. The corn plant or plant part thereof of claim 3, wherein the at least one non-natural mutation is a base substitution, a deletion and/or an insertion.

8. The corn plant or plant part thereof of claim 3, wherein the endogenous gene encoding the GRF transcription factor comprises a miR396 binding site sequence that, when optimally aligned to SEQ ID NO:252, comprises the at least one non-natural mutation in two or more positions 11, 19, 21 and 22 corresponding to the nucleotide sequence of SEQ ID NO:252.

9. The corn plant or plant part thereof of claim 3, wherein the endogenous gene encoding the GRF transcription factor comprises a miR396 binding site sequence that, when optimally aligned to SEQ ID NO:252, comprises the at least one non-natural mutation at position 11 corresponding to the nucleotide sequence of SEQ ID NO:252, and the least one non-natural mutation is a base substitution from G to A.

10. The corn plant or plant part thereof of claim 3, wherein the endogenous gene encoding the GRF transcription factor comprises a miR396 binding site that, when optimally aligned to SEQ ID NO:252, comprises the at least one non-natural mutation at positions 21 and 22 corresponding to the nucleotide sequence of SEQ ID NO:252, and the least one non-natural mutation is a base substitution from G to A.

11. The corn plant or plant part thereof of claim 3, wherein the endogenous gene encoding the GRF transcription factor comprises a miR396 binding site sequence that, when optimally aligned to SEQ ID NO:252, comprises the at least one non-natural mutation at position 19 corresponding to the nucleotide sequence of SEQ ID NO:252, and the least one non-natural mutation is a base substitution from G to A or C.

12. The corn plant or plant part thereof of claim 3, wherein the mutated gene encodes an amino acid sequence of one of SEQ ID NOs:43-47, 255, and 256.

13. The corn plant or plant part thereof of claim 1, wherein the mutated gene produces a mRNA having reduced miR396 binding compared to a control mRNA produced from a gene comprising a sequence of one of SEQ ID NOs:1-3.

14. The corn plant or part thereof of claim 1, wherein the corn plant and/or a corn plant regenerated from the part thereof exhibits increased growth and/or an increased growth rate compared to the control corn plant that is devoid of the mutated gene.

15. The corn plant or part thereof of claim 14, wherein the increased growth and/or increased growth rate is in roots and/or fruit of the corn plant and/or the corn plant regenerated from the part thereof.

16. The corn plant or plant part thereof of claim 1, wherein the corn plant exhibits increased growth as compared to the control corn plant that is devoid of the mutated gene.

17. The corn plant or plant part thereof of claim 16, wherein the corn plant exhibits at least one of the following phenotypes of increased meristem size, increased seed size, increased biomass, increased leaf size, increased root size, increased nitrogen use efficiency, increased disease resistance, increased height and/or increased internode length as compared to the control corn plant that is devoid of the mutated gene.

18. A guide nucleic acid that binds to a target site in a Growth Regulating Factor (GRF) transcription factor gene, the target site comprising the nucleotide sequence of any one of SEQ ID NOs:4-18, 121-144, or 173-198, optionally, wherein the guide nucleic acid comprises a spacer having the nucleotide sequence of any one of SEQ ID NOs:48-71.

19. The guide nucleic acid of claim 18, wherein the GRF transcription factor is a GRF1, GRF2, GRF3, GRF4, GRF5, GRF6, GRF7, GRF8, GRF9, GRF10, GRF11, GRF12, GRF13, GRF14, GRF15, GRF16, GRF17, GRF18, GRF19 or GRF20.

20. A system comprising the guide nucleic acid of claim 18 and a CRISPR-Cas effector protein that associates with the guide nucleic acid.


SEQUENCE LISTING (304 sequences)

SEQ ID NO: 1 - DNA, 22 bp, Zea mays
  source 1..22: mol_type = genomic DNA
ccgttcaagaaagcctgtggaa

SEQ ID NO: 2 - DNA, 22 bp, Zea mays
  source 1..22: mol_type = genomic DNA
tcgttcaagaaagcatgtggaa

SEQ ID NO: 3 - DNA, 22 bp, Zea mays
  source 1..22: mol_type = genomic DNA
ccgttcaagaaagcatgtggaa

SEQ ID NO: 4 - DNA, 62 bp, Zea mays
  source 1..62: mol_type = other DNA
cacatgcaccgcggccgcaaccgttcaagaaagcctgtggaaacgcagctcgcgccccagtc

SEQ ID NO: 5 - DNA, 62 bp, Zea mays
  source 1..62: mol_type = other DNA
cacatgcaccgtggccgcaaccgttcaagaaagcctgtggaatccaagaccgcctcctcgcc

SEQ ID NO: 6 - DNA, 62 bp, Zea mays
  source 1..62: mol_type = other DNA
cacatgaaccggggacgccatcgttcaagaaagcatgtggaaggccagcctggccatgccgc

SEQ ID NO: 7 - DNA, 62 bp, Zea mays
  source 1..62: mol_type = other DNA
cacatgaaccggggacgccatcgttcaagaaagcatgtggaagaccagcctggccatgccgc

SEQ ID NO: 8 - DNA, 62 bp, Zea mays
  source 1..62: mol_type = other DNA
cacataaaacgtggttgccaccgttcaagaaagcatgtggaaggccgaaaggcaacaccgac

SEQ ID NO: 9 - DNA, 62 bp, Zea mays
  source 1..62: mol_type = other DNA
cacatgcaccgcggccgcaaccgttcaagaaagcctgtggaagcgcagctcgtgcccccgcc

SEQ ID NO: 10 - DNA, 62 bp, Zea mays
  source 1..62: mol_type = other DNA
cacatcaataggaaccgccaccgttcaagaaagcctgtggaaaaccaacctagaaaggccac

SEQ ID NO: 11 - DNA, 62 bp, Zea mays
  source 1..62: mol_type = other DNA
cacatccaccgcgggaagagccgttcaagaaagcctgtggaagtgacctcccccgccgccta

SEQ ID NO: 12 - DNA, 62 bp, Zea mays
  source 1..62: mol_type = other DNA
cacatgcaccggggcaagaaccgttcaagaaagcctgtggaaatgtccttggccacgccggc

SEQ ID NO: 13 - DNA, 62 bp, Zea mays
  source 1..62: mol_type = other DNA
cacattcaccgcgggaagagccgttcaagaaagcctgtggaagtgacctcctcccccgccgc

SEQ ID NO: 14 - DNA, 62 bp, Zea mays
  source 1..62: mol_type = other DNA
cacatgcaccgcggccgcaaccgttcaagaaagcctgtggaagcgcagctcgcgcccccgcc

SEQ ID NO: 15 - DNA, 62 bp, Zea mays; identical to SEQ ID NO: 12
  source 1..62: mol_type = other DNA

SEQ ID NO: 16 - DNA, 62 bp, Zea mays
  source 1..62: mol_type = other DNA
cacgtccaccgcggccgcggccgttcaagaaagcctgtggaagccgccgcggcaccggcggc

SEQ ID NO: 17 - DNA, 62 bp, Zea mays
  source 1..62: mol_type = other DNA
cacatcaacagaaaccgtcaccgttcaagaaagcctgtggaaaatcaacctaagaagaccac

SEQ ID NO: 18 - DNA, 62 bp, Zea mays
  source 1..62: mol_type = other DNA
cacatgcaccgtggccgcaaccgttcaagaaagcctgtggaatccaagaccgcctcgtcgtc

SEQ ID NO: 19 - DNA, 1646 bp, Zea mays
  source 1..1646: mol_type = other DNA
gagcgaagcaaagcacatcacgagcccagcctgcgcctgcggagggagggggctcattaaagagggggcgcgagcgcgaccggccgcggggagcaagcagcgcgcgagagagacaggttgagatggcgatgccgtatgcctctctttccccggcaggcgccgccgaccaccgctcctccacagccacggcgtccctcgtccccttctgccgctccactccgctctccgcgggcggcgggctgggcgaggaggacgcccaggcgagcgcgaggtggccggccgcgaggccggtggtgccgttcacgccggcgcagtaccaggagctggagcagcaggcgctcatatacaagtacctggtggcgggcgtgcccgttccgccggatctcgtggttccaatccgccgcggcctcgactccctcgcaacccgcttctacggccaacccacactcgggtacggaccgtacctggggaggaaactggatccggagcccggccggtgccggcgaacggacggcaagaagtggcggtgctccaaggaggccgccccggactccaagtactgcgagcgccacatgcaccgcggccgcaaccgttcaagaaagcctgtggaaacgcagctcgcgccccagtcccaaccgcccgccgccgcagccgtctccgccgctccgcccctggcagccgccgccgccgccaccaccaacggcagcggcttccagaaccactctctctacccggccatcgccggcagcactggtggtggaggaggagttggcgggtccggcaatatctcctccccgttctcctcgtcgatggggggatcgtctcagctgcacatggacagtgctgccagctactcctacgcagctcttggtggtggaactgcaaaggatctcaggtacaacgcttacggaataagatctctggcggacgagcacaaccagctgatcgcagaagccatcgactcgtcgatagagagccagtggcgcctccccagctcgtcgttcccgctctcgagctacccacatctcggggcgctgggcgacctgggcggccagaacagcacggtgagctcgctgccgaagatggagaagcagcagccgccctcgtccttcctagggaacgacaccggggccggcatggccatgggctccgcctccgcgaagcaggagggccagacgctgcggcacttcttcgacgagtggcccaaggcgcgggactcctggccgggcctctccgacgagaccgccagcctcgcctcgttccccccggcgacccagctgtcgatgtccatacccatggcgtcctccgacttctccgtggccagctcccagtcgcccaacgatgactaatggtgcgtggatcgtcgcgttctggccctttgtctgtcttccctccagtcctccacccaccgcgcagtagtagctgcggaaacagcccatgctcctgtatatttgtcggtcattttccgtgtcagatctgtgtaccaaaccaagcggcggcggccgtcgtctctccccgcctccgcctcgcctcatgtgggtgggtgaacatgagaccgttttgcttaggctcaaagctttgtgtgaccccactgatcggttgtctgcgtactacctgaaagcctgctgcttttgtagcattactgt

SEQ ID NO: 20 - DNA, 1718 bp, Zea mays
  source 1..1718: mol_type = other DNA
aacccccacctgaccttctcttcctccctcccccgaccgcctgtctgtcctccgcgcatccgttctctatcgaaagggaggaggaggagcgcgcgggagtgggctgggggcccaccgatgctgagctcggcgtcctcggccggggcggccatggggatgggcggcgggtaccaacaccagccgctgccactgccgcagcgcggggcggcggccgcggtcttcaccgccgcgcagtgggcggagctggagcagcaggcgctcatctacaagtacctcatggccggcgtccccgtcccgcccgatctcctccgccccgccccccacgccgccgccttctccttcgccagccccgccgcgtcgcccttctaccatcaccaccaccaccacccgtccctgagttactacgcctactacgggaagaagctggacccggagccgtggcggtgccgccgcaccgacggcaagaagtggcggtgctccaaggaggcgcaccccgactccaagtactgcgagcgccacatgcaccgtggccgcaaccgttcaagaaagcctgtggaatccaagaccgcctcctcgccgccccagctgtccaccgtcgtcaccaccaccaccacccgggaggccgccgccgcgacgccccttgagtccctcgcgggggcggggggtaaggctcacggcctgtccctcggcggcggggctggctcgtcgcacctcagcgtcgacgcttcgaacgctcactttcgctatggcagcaagtaccctcttggagctaaatccgatgctggcgagctgagcttcttctcaggagcaccagggaactccaggggcttcaccattgattctccagcagataactcttggcactccctgccatccaacgtgcccccgtttacactgtccaagggcagagactctggcctcctgcccggtgcctactcctactcccacattgaaccaccgcaggtgctcggccaggtcaccatcgcctcgctgtcacaggaccaggagcgccaccagtcgtcgttcagcagcggcggggccggcgctggtggattgctggggagcgtgaagcaggagaaccagccgctgaggcccttcttcgacgagtggcctgggacgcgggactcgtggtcggagatggacgacgcgaggtccagtaggacctccttctcgacgacccagctctccatctccattccgatgcccagatgtgattgagaacgaagctcgtgcgcggccagtaggtggcgttgtcgcatgacgcgcacccagcatcgcgctcgctgctagcgagccaccgtcacccggtgattgatgccctcatcctagctatttgtttcgctgaatcgcattaagaacagaactctatgtaagagcaggatccttcctcatgtatcaatgggggcaatgaactccattctgtttctatggttgtagtaccgaccatgctttgtatttgttgtacccagaccgagaatgaaaaccgcccagtcgtcaggcgttgtcgtcgctgcatcagatgatccaagtgtattgtgtgctttccaggctgtgtaagatctgatggcgaatctgttactgcagtgtaggtgtgtagcatctgtggtatggtaccttttgttgtgctactggatctggattgatgcaaaatctggtgctgccgtaggcccctcttccttttctctactgctataataataatacatcttttttttatg

SEQ ID NO: 21 - DNA, 3536 bp, Zea mays
  source 1..3536: mol_type = other DNA
accgagcacacatacacactcaccagcaggcagcagcagctagccaagctataccactagaaccagagcacaactcaaagcaatgccgccgcctcctcctcctccgccccactcccacgcttcccatcccgcgccgcatggacgcgagcagcactagcgcgggggcacacagcacggcgagcgggcgggggcgcgacgatggacctgggcgggtcggtggtgatggacgcgggcgcctcggagctgggcctgctcggcggcggatctagcaggctgctcaagcacgggcgggggcccaatgcggcggggggagacgacgacgtgtggggcggcggcagggccaagcagcccaggacagggacagcgcccgtcgtcgccggggacgtggcggctgccaccaaggcggcggcggcgccgttcctgctgggctcgtgcagtcccggccacggcggggagcagatgctcagcttctcgtccgccgccgccgccgccgcggcctcgtgcgcctcctccaccgctgcggctgatggcggcggggccatgccgctgtactacgggaccccggcctcttgctcaggtgagcgcgagcacggcgacggcgtacccgtgctactgcttgttgtggcggtgccgcttgctgctgcaaagcttacttctttttttcccgttgcttgttgattggattttggtgattgaaaagcttgctatccacttcttctccagtattattgcttttcggttcgattcgattcaccctgctttctctctttgctcttggtctcgtagctacagatttatttatttttacttggttatgtctactgactgactactgcttatccttaagctgatctgtgccctcttccgtttcttctcctcttcttctttcttctagcttcagttatttagtgcatctactctagattcatggggttttcatgaccagtgttgactcttttaacctgccaatgatctttgtatggggttttcctctctcttttcgtcttctggagatttagattagtgacgactgtaaaagttcttccacctctggaaaaaagaaatggttttaaatagtattttaaaaaaagctttattgaatcatccgtgatatgtatcgacataagtttgcacttctttttgctgacatccgccaacgcttttcttgtcctcaacggcgccgcgatcgtgtggattcctttttgtcacgccgcctgctgataatacgactactagttcttattagaggaagaatgaaaaagaatggcgtgatctacctcggacctataccttcacccctttctcactcagaaacctaaaaaccaagaaacagctaaaagagcatgaatgcacgcaagaaagtctcaagaatacaatggagcctcgcgattcaagttcactgtcgggacccaacagtgtggtccttggtcggcttgctatggacatcacctataaatctgtgctgcttatcaggtttctgatccttgcatgcaggcgctccgttccaggtttttgtgtctttttttgcttgccttttatcctgccctaggatcgtgtttaggaaacgcttgcatctcggcggtcatctctatgaagcagtacacatgtttttggctgcgccttgttggcttccaccgttttgctgctcatctctcctaactatgcctgcctgtttgctccccgcaggattgagctcggtgagcttgagcaccagcatgcagggggcaatggccagggtgagggccccattcacgccgtcccagtggatcgagctcgaacaccaggccctgatctacaagtacctggccgcgaacagccccatacctcacagcctcctcatccccatcaggaggagcctcgcagcgtctccctacccgccttcatacttcggcacgagcacattggggtggggctctttccagctgggctactccggcaatgcggatctggagcctgggcggtgccgccggacggacggcaagaaatggcggtgctccagggacgcggtcgccgaccagaagtactgcgagcgacacatgaaccggggacgccatcgttcaagaaagcatgtggaaggccagcctggccatgccgcgaaagccatgtccgcggtggcggcggcggctgctgctgctgcccagcctgctgctctggccggcgccggagctcctgctgctggcctcactgtcaatcagcaccagctgccggcgaagagctacgccacgggtgcgactgacccttgctctctgcaatataacagggaactggtgaacaaacagaatgagagcgaaaacatgcaggactccgacaacctctcgatgctgacttccatgaacacaggaaacacaggcagcgtgttcccattctcgaaacaaaacaccaatccttttgaagtgaccagctcaaggctagagttcggcctcgtttcatcggattcactgatgagttcccctcacagttccttggagaacgtcaacctgctcggctcacagagtatgaacgaacaccagaacccagcctccctgcaacacttcgtggactggcccaggacgcctgcccagggaggcctttcatggcccgaagctgaagacatgcaggctcagaggtcacagctctccatatctgctcccatggcatcctccgagctatcgtcggcctccacatcccccatccacgagaagctgatgctgtcgcctctgaagctgagccgcgagtacagcccgactggtctcggcattgcggcgaacagagacgaggctagccagctagaagcaacctgggcgacgatgttccgtgactcttcgatgggcggtccgttgggggaggttctggccaagaacggcaacgtggaagcgagaaattacctgtcggcgcctctgaaccttctcaccgactactgggattcaagccacgggatggagtcctcccctgtgggcgttctgcagaagacgaccgccttcggatcggtctccagcagtacagggagcagccctagggttgagaaccacggcgcctacgacggtttcggcaacctgcgggatgatctcggctcaattgtagtgcgccatccaagcatccgccttgtgtgagctttctgagttctgaccatggaaaactgcaacaagacggcggcccgcagccatagaagtgggcatatgggctactatgctaatcctcaaagcgtgaggggggggcatttacctcgtagtttgtcatttgagactgatctcctgttttttgtcacttcagttcttcttttgcttccctttttaacttggctttgtaactttcaccttccttggatcgtgaaaagcgctgtggtgtgtggtgtggatggatttggtgcgccgctgatgcatatcatcatgtgatagaagaagccagtatccgtcgaggattactgcgagtatcgcctgtttgttgttcatacgtcatactgctgcactgcacttcactgtttgtgtgcccgttgttgcattgtatggttcggttatattaatttacaaacaggtcgtgaggctgcacatttagattcg

SEQ ID NO: 22 - DNA, 2086 bp, Zea mays
  source 1..2086: mol_type = other DNA
cgagcaccgcgcgcgcacagacacacactcaccagcaggcagcagctagctagctctctagcaacgagcacaaaacgagcaatgccgctagttccgtcccactccctcgcttcccatcccgcaccccatggaagggagcagcactagctagcgtgggacagggcggggtggtgtcgtggtgatggaagcgggcggcggcgcctcggaactcggcctgctcggcgccggatctggcaggctgctcaagcacgggcggggcgacgcggcggggggagaggaccacgggtgggtcgtcgtcgtcggcggcggcggcagggccaagcaggccaggaccgcgtccgtcggcaccggggacgcggcggccacggcggccgtgccgttcctgctgggctcctgcggcggccccggccacggcggggaacggatgctcagcttctcgtccgccgccgccgccgccccggatgatggcggtgccatgccgccgctgtactacgggaccccggcctcttgctcaggattgatgagctcggcgagggtgagaggccccttcacgccgccgcagtggatcgagctggagcaccaggccctgatctacaagtacctgtccgcgaacagccccgtacctcacagcctcctcgtccccatcaggaggagcgggagcctcgcatcgtctccctacccgcctccctacttcggcacgagcacattcgggtggggctccttccagctgggctactccggcaatgcggatctggagcctgggcggtgccgccggacggacggcaagaagtggcggtgctccagggacgcggtcgccgaccagaagtactgcgagcgacacatgaaccggggacgccatcgttcaagaaagcatgtggaagaccagcctggccatgccgcgaaagccatgtccgcggtggccgtggcggctgctgctgctgctgctacccagcatgctgctgctctggccggcgccggagctactgctgctgctggcctcgctgtcaatcggcaccggcagccggggaagagctatgccacgggtgcgactgatccatgctctctgcaatatagcagggaactggcgaacaaactgtcgaacgagagcgaaaacatgcaggactctgacaatgtctcgatgctgacttccatgaacacaggaaacactgcaggcagcgtgtttccgttctcgaaacagaacactgctcttttcgaagtgagcagctcaaggccagagtttggccttgttccatcggattcactgacgagttcacctcatagttccttgaagagtgtcaaaaatctgctcagctcacggagtctgagtgagcaccagagctccgcctccctgcaacactttgtggactggcccaggacgccacctgcccagggaggtctttcatggcccgaggcagaggacatgcccagagctcagaggtcccagctctcagtatccgctccaatggcatcctccgagctatcatcggcctccacatctcccatccacgaccacgagaagctgattctgtcacctctcaagctgggccgcgagtataccccatctggcgtcgtcagcgctgcagcgaacagatacgaggttagccagctagaagcgacctgggcgacgatgttccgcgactcttcgacgggcggcccgttgggggaggttctggccaagaacaacagcaacgcggaagcaagaggttgcccgtcggcggcgccacctctaaccgactactactgctgggattcaggccacggcatgcagtcgtcccccgtgggagttctgcagaggacgaccgccttcggatcggtctccagcagcacgggcagcagccctagggtcgagaaccacggcgcctgctgcgatggtaccagcaacctgatggatgatctcggttcagttgtagtgagacatcccagcatccgcctcttgtgagcccgctgggttctggccatggaaaaagcgtgagggggagcatttaccacgtagtttgtcatttgagactgatctcctgtttctgtcacctgagttcttcttttgttgcccttatacctggctttgtaactctcaccttcgttggat

SEQ ID NO: 23 - DNA, 2328 bp, Zea mays
  source 1..2328: mol_type = other DNA
aggggcaagctataattttgttctgttgtccagaacagccaacaggtgtggggctgtgggcattcaattgcttttgcttcctccgttcccccatctggccatctccccttcccctgctcccccgaagcagcaagccagcctgcccacccgcagccatcacctccgccgctctccaccatgaatcccatccaccagcacggcatcgtacccaatccttcgtgactgttgcctccgcgcatctccgggagcaatggaaggaggccgagatgtgttcttaggtgcggcggcaagggcgccgccgccgccgccgtcttgcccgtttcacggatccgctaccgccacccgctccggtggagcgcagatgctcagcttctcctccaatggcgtagcagggttgggtctgtgctcaggtgccagcaagatgcagggtgtgttgtcgagggtgaggaggcccttcactccgacgcagtggatggagctggagcaccaggccctgatctacaagcacttcgctgtgaatgcccctgtgccgtccagcttgctcctccctatcaaaagaagcctcaatccatggagcagccttggctccagctcattgggatgggcaccatttcgttccggctctgctgatgcagaaccaggaagatgccgccgcacagatggcaagaagtggcggtgctctagagatgctgtcggggaccaaaaatactgtgagcgacacataaaacgtggttgccaccgttcaagaaagcatgtggaaggccgaaaggcaacaccgaccactgcagatccaaccatggctgtttctggtggttcattgttgcacagccatgctgttgcttggcagcagcagggcaaaagctcagctgctaatgtgactgatccattctcactagggtccaacaggtgaagtcacctgatcgtctgatcctgcattgttatactcatttctgccacttaagtttgtgaatttttattttctcaacatttttttctcgaacacgcaggagaactgtgcatcatatattaaaagaagtaaaaaagtctaaagaagaccaaaatctgccataagaggcagaaagagataggaagtgggaggggcaccagcacactatattttctcaacataaatgacaatctatggttgatacatgtgccagtctagcagtcagggttcaggacgtaccaaaacattgaaaatgttatatcctttagacattgaaattttgtccatagccattcatagttgaaaattttgttcttgatttctttcaactggtcagtgatttcttcatgtaacctgtcattgatgcattccatcttttgccccgtgcccattcctatatataagattttattattatgtttgtttgttcaaacaattatggtaagcatgtgcaaagaaaggcagtaggaagcactataaataactgaaatgatatggtacttatgtcagtacaaactttagagataggtgaattctgttcatgatgaccctttcaaattttgacgacttactatagtcagcagtgaaagaaaaacaggacagtgtttccttttccttccatgaacttgaaatgttgactctggtttgcaggaatttgctggataagcagaatctaggtgaccagttctctgtatccacttccatggactcctttgacttctcatcatcacattcttccccaaaccaagccaaagttgcattttcaccggtggccatgcagcacgaacatgatcagctgtatcttgtgcatggagccggcagctcagcagaaaacgttaacaagtctcaggatggtcagctgctagtctcgagggaaacaattgacgacggacctctgggcgaggtgttcaagggcaagagttgccagtcagcatccgcagacatcttaactgaccattggacttcgactcgtgacttgcgtcctccaaccggagtcctacaaatgtctagcagcaacacagtgccagcagagaatcacacgagtaacagtagctatctcatggcgaggatggcgaattctcagaccgtcccaacactccactgagtgttcatcaggctggtctttgttgggaccacaaaataactgaagccatgttgatgtcctgagtttgctgatactaggttttcagtcgagtcttgtaactcctgttttagagctgttatatgttcacgtcatgtcgcctttcattttcggttcattcagatgggtgttctaataatttctttccttcttacctgtgaaggatttgagttccaatctgagacgtgggtttgttctaacttggaggtatttatgaatattaggcacttctgatttccat

SEQ ID NO: 24 - DNA, 2279 bp, Zea mays
  source 1..2279: mol_type = other DNA
gagagagagagaactagaagcatatatggcgatgccctttgcctccctgtctccggcagccgaccaccgcccctcctccctcctcccctactgccgcgccgcccctctctccgcggtgggagaggacgccgccgcgcaggcgcaacagcagcagcagcacgctatgagcggcaggtgggcagcgaggccgccggcgctcttcaccgcggcgcagtacgaggagctggagcaccaggcgcttatatacaagtacctcgtcgccggcgtgcccgtcccgccggacctcctcctccccctacgccgaggcttcgtctaccaccaacccgcccgtaagcaagcacggcccccgcgccgcctccgcaccccttcacactcacacgcacgtttaaccgcttttgcactgcacaaccccggccgcccggcggcggcgtccgtgccttgatctggttgtttactcggatcgagggattcagatgtcctctccgtccgtttgttaatcggctccggtcatttcttaatctcgtcctggattcggtcacgaaaagctagaggtcaagattttgctctcgattactatatccttgcctcatgttctaatggagtttattttattggtctgatgtgattagataggatgctagccaggcttgtctccggccaaaagcggcggtttagtttattgatgattgcttctttccttgggggatttattcctgtctggttgttgggagcctaaccacgctcctattgctgctgcggtttactaaccatctgcgccagtacacctactccatggaccccaaaatacagttcttccaaccattccccccctccatctgctttctcgcgggcaaataaaaacgtgtagaacgacggtgtagtaggcagatctactccttgtgccgctacgctagcccgctaccgaagatcgggcccgtttcaaccggttcgttggtctgagcggagctaagatggggcgcatttcattttttggtcctttcgtctgattggagaagtgcccattccggtatcgctccccggcctccaaatacgcaccgacacagaacgtgttcgtacgcacgtacacatggtatgcgcaccgtgctgctggccatagccgttgactcaccgggattcactcctctctcgcgtgtgtgtgtgtggcttccttgcagttgggtacgggccctacttcggcaagaaggtggacccggagcccgggcggtgccggcgtacggacggcaagaagtggcggtgctccaaggaggccgccccggactccaagtactgcgagcgccacatgcaccgcggccgcaaccgttcaagaaagcctgtggaagcgcagctcgtgcccccgccgcacgcccagcagcagcagcagcagcaggcccccgcgcccaccgctggcttccagagccaccccatgtacccatccatcctcgccggcaacggcggcggcggcggcggggtaggtggtggtgctggtggcggtggcacgttcggcctggggcccacctctcagctgcacatggacagtgccgctgcttacgcgactgctgctggtggagggagcaaagatctcaggtactctgcctacggggtgaagtctctgtcggacgagcacagccagctcttgtccggcggcggcggcatggacgcgtcaatggacaactcgtggcgcctgttgccgtcccaaaccgccgccacgttccaggccacaagctaccctctgttcggcgcgctgagcggtctggacgagagcaccatcgcctcgctgcccaagacgcagagggagcccctctccttcttcgggagcgacttcgtgaccccgaagcaggagaaccagacgctgcgccccttcttcgacgagtggcccaagtcgagggactcgtggccggagctgaacgaggacaacagcctcggctcctcggccacccagctctccatctccatccccatggcgccctccgacttcaacaccagctccagatcgccgaatggaataccgtcaagatgaacctgagtaaccatgcggaccccaacatctcagagctgacgactctttgctgctggcctggcctcatcgtaccttgaggcgtcaaggaatacttcattaccactagtatcatgctcctggattttcgaacaatatatatatgcttatgtaccgctatttctctcatcttttacacttctttacccgtttggaattgtatgttctgcgtggcacggttgttcatttgacctttttggatttgattgaaagctccgtttcttgc

SEQ ID NO: 25 - DNA, 1790 bp, Zea mays
  source 1..1790: mol_type = other DNA
ccaccacacaaacggacaaacccctgcctcccaccagaaaagcaagcagaccacacccacccgcctctaaaccaaccagcctgtccccattccccgtccccggcgatgcacgccagcatccactccctcccgtcagcctcttcttctccccctgatgagcgctgagttctgtgctgccgctgctggtgctgtggccatggagctcggagtcggggatgtgatggggctgcagcaaggcatcgccgccgccaccgggccatcgtccggagacagcgacctgggtcttctcaagcgagcaggcctcgcccaggcagccacctcctacccctcccctttcctcgaccaacagaagatgctcaggttctccaaggcggcggcggctcacacgtcgccctcaggcctagacttcggaggaggcccgagcgagcaggctttcctgctgtccaggaccaagcggccgttcactccgtcgcagtggatggagctggagcaccaggctctcatatacaagtatctcaatgccaaggcccccataccttccagcctgctcgtttccatcagcaagagcttcaggtcatccaacagagagcttgacagactctctaacgcagtgagctggaggcctctttaccaaggctacgcaaacgcagactccgacccagaacctgggaggtgccggcggacagacggaaagaagtggcggtgctccaaggaggcgatgcctgatcacaagtactgcgagcgccacatcaataggaaccgccaccgttcaagaaagcctgtggaaaaccaacctagaaaggccaccaaggaggtgactaccgctgctgctggctcgttgccgtgtgccgggccacaaggtagcttgaagaaggcaaaagttaatgactccaagccaggcactggcagctattggacagatagcttaaacaggacaatgctgagcagagagaaggcaaacaaaccgacggacgacgagtctttgctgcttagttcgacgaagaacagtcagcccaccttgtccctgctcactcaactgaagcagcagaacaaaccagataagttaggtcccacaccggaaaatgagccgaactcggacacgatgttgaaagcctggggtggcagccaccacaagagcatttcctccacacagcgccatgacgctgaatccctccaatcagtcctccaaaatttcagcctagcccagaatgacaggttggagtcagaaaagaacagatattctgattccgtgctagtctcatcggctttctattctgcagacggtccacaaactacctgccttacacctaacatgacacaagtgcagcaggactgcatatcaagctcctgggagatgcctcaaggtggacctctaggcgagatcttaacgaactccaagattagtgaggacttaagcaagtgtggatctaggtcatatggttggctattgaatcttgaccatgcaccatgattcctcaatccatggagagcttgacatagatatctcaccacggatgcaaacaatgagtcaggaaaaaagaaggtccaaatgaccgtactgtttaccccatgcatgctcgttatctacatttgtacttctatttttgtagtattcagctgttgaattatcagttcttctgaatctggctgtatcttaaacaaattctagtttccgtcaaatgatatcttgcttgctagatgcttaatgtttccttttcagcagaaacttcagtggtccattttaatcaaaagaaaactgtttcaagatcaagctctagtaagcaaagttc

SEQ ID NO: 26 - DNA, 1725 bp, Zea mays
  source 1..1725: mol_type = other DNA
gccgactgcaccccacaccacccccccacctcagctcaccttgcgctgcccctgtgcctgctgcatgcctgacaccggcgccatatgcatgatagcggatgatgatactagcacgtcttcccgttcccgagctgcctctgacaccagcacaaacctggagactactactagtattggagtcccctccatttccacctcccttgccactgaagcgagagctctcggagccgtcgtcctctgtctctcatccttcttcgttgttgagcaaagcgggctcgaggaggagatgatgctgagcgggcacggcggcgggaggcgcctgttcacggcgtcgcagtggcaggagctggagcaccaggcgctcatcttcaaatacatggcctccggcgcgcccgtgccgcacgacctcgtcctgccgctccgcctcgccaccggcgtcgacaccgcgccctccctcgccttcccgccccagccttcgccgtcgctggcgtactggggctgctatggcgcgggggcgccgttcggccgcaaggcggaggacccggagcccgggcggtgccggcggacggacggcaagaagtggcgatgctccagggaggcccacggagactccaagtactgcgagaagcacatccaccgcgggaagagccgttcaagaaagcctgtggaagtgacctcccccgccgcctaccgcccgtccgcgttctccatctcgccgcctcgcgcggccgacgcgccgccgccgccgccgggcctcggccacccgcagcagcagcatctccgccacggcgctctctctccagcaggccgcgcccacgccgctggcgctctccagctccacctcgactcgagcctgcacgcggcgtcgccgccgccgtcctaccacaggtacgcccactcccacgctcactacacgccgccgccgccgccgtcgctctacgactacgggcagtccaaggagcttcgggaggcggcggagctcaggcggcggcacttccacgcgctcggggccgacctgagcctcgacaagccgctggccgacgccggggccgcggagaagcccctgcggcgtttcttcgacgagtggccgcgggagagaggcgacacgaggccgtcgtgggcgggggcggaggacgcgacgcagctctccatctccatccccgcggcttcgccctcctctgaccacgctgcctctgccgccgcgcgatgccacaacgatgggagtgatcggtgcatctcctagctgcaactgcaatgcaagcctgcaaccgcgtggattgttgttgattggtgtagtttcctagctgcaattcaagcctgcaacagcgagcagtgagcagcaaatgcgtggggagggcacgcagctcaggctgatgcgcaaaatccgaagcgagtcaagcagcaataggactctaggtctatgatttgatcttcctttgtagcagtacgttaccaaaatgttagctcgttgttgttcggtgtgacattttcgttcaggttgctcctattggctgctgcaactgcaacgctagctctgtggcttttgtgtgctttcagtctgcggtgcgagccttgtcagctcttttccggtgacccacgcctttgattcctttctctgattcctctgcgggctctgtcattttgatcgtttgtcacatccgtggattcttcctcgcgggactactcccgtcacgaaatataaacaaacgcacatggctttctttc

SEQ ID NO: 27 - DNA, 2475 bp, Zea mays
  source 1..2475: mol_type = other DNA
cacaatcacacgcgacgcggcaggcgtacgccgtcgccgcgtctccgtccgtacccgccgctggtctcgtggaccgtgcggtgggcgggtgcgacgcgactgtgagagcagcgggggcccggtcggctctcgtcgcgcggctgcagccagcactgcatgcaggcacagcagcagccgcggacacggcagattctgcagcgcgcaccctgcatgctgtcacggctgtgaatggcctgcgcagcgctggcagcctgtgccgccggagaactgtcccagacactgaccggtacatgtggagaatcacgtccaaggcactgaccggtcgcgctgccgcgcgtgccctccttgcttcgctcacctcacacacgtcgtacgccccacccgcatgctgcacggcacgtcccgtctgctcctgccaggctcccatgatgacaccgagcgagcggtacggaacacagcctactacagtgtctgacgcctgacggacgggttagcgtaagcagccagattcgcgtcccggagctctcgcctttgttgacgaggtgaggggggactgggcacggctgctgctcggtcgtgctgcccgttacagtagttggaggaacgcagcagggccgttgggtcggagcaccggtgcccgtcgttcgtgtggtcgatccaatggcccaaatctcttccgcggcccaacaaacccacacacgcaggcgcctgcgaggcttgctcgtgcgacccacagcagcctgctgccgggcgccgcccactacaaataaaaccccttccccatggacgtcgcccaagagactcactgctactcgcgctcttgccaccgtctactctccaacccttctgccatccctcggcaccagctcgctgacgctaccgccacccctaccgccaggccgcatagccgtgctccgctcaccttctctcgcgctacagtctcaagggtagctagccaagctaccaagctcgtcaggagcgagagaaagaggccgccggcggtgcgcggggatgatgatgatgagcagcggccgggcgggcggcggggccaccgcggggcggtacccgttcacggcgtcgcagtggcaggagctggagcaccaggcgctcatctacaagtgcctggcgtccggcaagcccatcccttcctacctcatgccgccgctccgccgcatcctcgactccgccctcgccacgtcgccgtccctcgcctacccgccgcaaccctcactgggctggggctgcttcgggatgggcttcacccggaaggccgacgaggacccggagcccgggcggtgccggcgcacggacggcaagaagtggcgctgctccaaggaggcgtacccggactccaagtactgcgagaagcacatgcaccggggcaagaaccgttcaagaaagcctgtggaaatgtccttggccacgccggccccggcgccggcccccgccgccgccacaaccgccaccgccacctcatccccggcgccgtcctaccaccgcccggcccacgacgccacgccgtctccgtaccacgcgctgtatggaggcggcggcggcggcggcggtagcccttactcggcgtcggcacgcccaggagcaaccggaggcggcggcgcgtaccaccacgcgcagcatgtgagccccttccacctccacctcgagaccacccacccgcacccgccgccgccctacaactactccgccgaccagagggactacgcgtacgggcacgcggccgccaaggaggtcggcgagcacgccttcttctcggacggcgcgggcgagcgggtcgaccggcaggccgcggcggggcagtggcagttcaggcagctcggggtggagacgaagccgggccccacgccgctgttccccgtcgccgggtacgggcacggcgcggcgtcgccgtacggcgtggagatgggcaaggacgacgacgagcaggaggagaggcgccgccagcactgcttcgttcttggagccgacctgcggctggagcggccgtcgtcgggccatggccatggccatgaccatgacgacgccgccgccgcgcagaagccgctccggcccttcttcgacgagtggccgcaccagaagggggacaaggccgggtcgtggatggggctcgacggcgagacgcagctctccatgtccatccccatggccgccaccgacctccccgtcacctcccgcttccgtaacggtgggcactacgagtgatgccacatcaaacctggcgctggaaactcggaacgtatggtgctgatgctgaccatgagatgtaccgtttcgctcaaggccggccgtcatcaccacggccacatcaaaccggtttgaagaacttgggaaggccgaatgaaacctccagaagaatgatctgtcctctttttctccattttcttttttcttttagtattactctgtattgcttcgcgtgtaatatgttttcctgtaatatcaagtgagggcgtttctgaa

SEQ ID NO: 28 - DNA, 1539 bp, Zea mays
  source 1..1539: mol_type = other DNA
atgatgctgagcgggcacggcggcgggaggcgcctgttcacggcgtcgcagtggcaggagctcgagcaccaggcgctcatcttcaagtacatggcctcgggcgcgcccgtgccgcacgacctcgtcctaccgctccgcctcgccaccggcgtcgacaccgcgccctccctcgccttcccgccgcagccttcgccgtcgcgtacgacctgcgcgcttctctcctcccgacccgacctcccccgtcctcgcctcatgtttcactctgatcatcgacagccatcagcccatcacatcatcgctgatgactttgtacgtgtgaccgtgcatgcagtggcgtactggggctgctacggcgcgggggcgccgttcggccgcaaggcggcggaggacacggagccggggcggtgccggcggacggacggcaagaagtggcggtgctccagggaggcccacggcgactccaagtactgcgagaagcacattcaccgcgggaagagccgttcaagaaagcctgtggaagtgacctcctcccccgccgccggccccgctgcggcgtaccgaccgtccgcgatctccaccatctcgccgccccgcgcggccgacgcgccgccgccgagcctcgcctacccgcagcagcatctcctccacggcgcctcctccgcagcaggagcagcagcccgcgtccccgctggcgctctccagctccacctcgacgcgagcctgcacgcggcggcggcggcggcgtcgccatcgccgccgccgtcctaccacaggtacgcccactacacaccgccagcgtcgtcgctcttcccgggcggcggctacggctacgactacgactacgggcagtccaaggagctcaggcgacggcacttccacgcgctcggggccgacctgagcctcgacaagccgctgcccgagcccgacaccggctccgacgagaagcagcccctgcggcgtttcttcgacgagtggccgcgggagagcggcgacatggccgcggacgacgcgacgcagctttccatctccatccccgcggcttcgccctccgacctcgctgctacctccgcctccgccgccgccgcgcgattccacaacggggagtgatcggtccatctcctagctgcagccctgcaacagcgtggattgaccgctgcatttcctggctgcaatgcaagcctgcaacagcgagcagtaagccagtgacgtggatgcatctcgtagcggcaaaccctgcttctgcctctgcggtctgcgctgctttgcgcgggacggctcaggggctgaggctcaaaaaagtctgaaccgagtcaagcagtactagggactctatgacttggtcttttgttcttttgtagcagtacgttactcgtaccaaatgttgtttattttttgtgtgacattttcatttcttccagttccagtctgtgatgcaagctttgtcgcttttgtcagctctttttcggtgacccacgccggccttcgtgatcgattcccttatacttatacctgattcctctgccatattgatcgtttgtc

SEQ ID NO: 29 - DNA, 1619 bp, Zea mays
  source 1..1619: mol_type = other DNA
ggaaccctcccgtgtcgacctcaccccaccgcgcagccctgcacggccgcggtcgcctataaggctagcccagccagtggtagcacgcacagcacagcagccgccctgctcggcgtaggagcagcccagagcgagcgagacagagggaaagagacacggccagccaggtaaggcaaaagagagagggcggaagcagcggcagagcggagagggagagagaagagcatatatgggcatggcgatgccctttgcctccccgtctccggcagccgaccaccgcccctcctccctcctccccttctgccgcgccgcccctctctccgcggcgggggaggacgccgcgcagcagcacgcgatgagcggcaggtgggccgcgaggccggcgctcttcaccgcggcgcagtacgaggagctggagcaccaggcgctcatatacaagtacctcgtcgccggcgtgcccgtcccgccggacctcctcctccccctgcgccgaggcttcgtcttccaccagccacccgcccttgggtacgggccctacttcggcaagaaggtggacccggagcccgggcggtgccggcgtacggacggcaagaagtggcggtgctccaaggaggccgccccggactccaagtactgcgagcgccacatgcaccgcggccgcaaccgttcaagaaagcctgtggaagcgcagctcgcgcccccgccgcacgcccagccgcagcagcagcagcaggcccccgcgcccgctgctggcttccagaaccactcgctgtacccgtcgatcctcaccggcaacggcggcggcggggtaggtgctggtgctggtggtggcacgttcggcctggggcccacctctcagctgcacatggacagtgccgctgcctacgcgactgctgccggtggagggagcaaatatctcaggtactctgcatacggggtgaaatctctgtcggacgagcacagcacgctcttgtcgggcggcatggatccgtcgatgatggacaactcgtggcgcctgctgccatcccaaaccaacacattccaagccacaagctaccctgtgttcggcacgctgagtgggctagacgagagcaccatcgcgtcgctgccgaagacccagagggagcccctctctttcttcggcagcgacttcgtaaccgccgccaagcaggagaaccagacgctgcgccctttcttcgacgagtggcccaagtcgagggactcgtggccggagctgggcgaggacagcagcctcggcttctcggccacccagctctccatctccattcccatggcgacctccgacttctccaacaccagctccagatcgccgggtggaataccgtcgagatgaacgagtaccgtgcatgtggatcccagcgtcttagggttgacgactcttcggtgctggcctcgtcgtatcatgctcctaaattttcgaacgatatatgccttatgtaacgctatttctctcattgttacaacaccctttacccgtttggaattgtgttgaagtggatggtctgcgttgctcggttgttcattcgaccttttttcaatttggaaactccgtttctttgtactcagtgatctgtgcaagtgcaatgattcggtccatg

SEQ ID NO: 30 - DNA, 1750 bp, Zea mays
  source 1..1750: mol_type = other DNA
cgcgatccaagcctgccactacaaataaaactcccttccccatgaagctcacctaagtgcttcactcactcctgtcctctcctctcgaccagctgacctcgctgacggagcacacagccacgccgcagcgccgcgtgggtgtctgtctatagccttgtaggtcgttcgcaggtaggtaaccgtaacctagctagctcgtcgggatgatgatgatgagcggtcgagcggccaccgcggggcggtacccgttcacggcgtcgcagtggcaggagctggagcaccaggcgctcatctacaagtgcctggcgtccggcaagcccatcccgtcctacctcatgccaccgctccgccgcatcctcgactccgccctcgccacgtcgccgtcgctcgccgccttcccgccgcaaccctcgctggggtgggggggctgcttcgggatgggcttcagcaggaagcccgccgacgaggacccggagcccgggcggtgccggcgcacggacggcaagaagtggcgctgctccaaggaggcgtacccggactccaagtactgcgagaagcacatgcaccggggcaagaaccgttcaagaaagcctgtggaaatgtccttggccacgccggcgccggcctcctccgccacaagcgccgccgccgccgccacctcctcgtcccaggcgccgtcctaccacagcccggcccccgccgtgccgtaccacgcgccctacggcgccgcgtaccatcacacgcagacgcaggtgacgatgagccccttccacctcctccacctcgagaccacccacccgcacccgccgccgccgccgccgccgccctactactacgcggaccagagggactacgcctacggcaaggaggtcggcgagcgcgccttcttctccgacggcgcgggcgagagggaccggcagcagcaggccgcggggcagtggcagttcaagcagctcgggacgatggaggcgacgaagcagccgtgcaccacgccgctgctcgtccccgccgccgggtacggccacggcgcggcgtcgccgtacggcgtcggtcaggccaaggaagacgaggaggaggaggaaacgcggcggcagcagcagcactgcttcgttcttggcgccgacctgcggctggcggagcggccgtcgggggcacatgacgccgccgcgcagaagccgctccggcatttcatcgacgagtggccgcacgagaaggggagcaataaggcggggtcgtggatgggggggctcgacggcgagacgacgcagctctccatgtctatcccgatggcggccgctgccgacctccccgtcacctcccgctaccgtacgtgatggcggcgcgcactatgagtgattgccagcaagaaatgttttgtggttgctgcgacctcgaggtggcgaggccacatgaaaaaaaaatactggagctggaacacgagtggttctttcctgtgccgttttgctcagggccggtcggcacggtttgaagaagttgggaagagaagtaaaataaacctccttgggagtgttacgtttcctgtaattgcttgcttcctttgctgtacctgtgagacgacgcttagtgctagaagtgagggcctttcctgatctcagctctggtctggatttgccttttcccgctgtcggattcctctgcctgtctgtgatggtttgtctgtgatggtttgtgctcagagcctgcagcgaatactaaaccgcagtaattaatcaccatgcttcacccgaca

SEQ ID NO: 31 - DNA, 852 bp, Zea mays
  source 1..852: mol_type = other DNA
cttcttccctcctctcgagaagctgcctgctacccgcgcgctgcctctcccttccactccataccataccgccacgacaacgtcgacaagcgcaataccggagccctccgaaatggccgaggacaaggagaccgactcgccgcagccgccggccaagctgccccgcctctcccgcgccgacacgagcgccggagaggtgaccatggcggcctcgtcgccgctggttcttgggctggggctgggcctcggcgggcgcggcggcggcggcgagcgagacgcggatgtgtcccccgcgacggtgactcccaagaggccgtcggcgctgacgttcatgcagcagcaggagctggagcaccaggtgctcatctaccgctacttcgccgcgggcgcgccggtgccggtgcacctggtgctccccatctggaagagcgtcgccgcgtcctccttcggcccgcagcgctttccctcgctgatgggcctgggcagcctgtgcttcgactaccgcagcagcatggagccggagcccgggcggtgccggcgcacggacggcaagaagtggcggtgctcccgcgacgtggtgccgggccacaagtactgcgagcggcacgtccaccgcggccgcggccgttcaagaaagcctgtggaagccgccgcggcaccggcggcggcggccggcggcagcagccccgtcctccgggtcgccgcgccccagcacgtgctcggcctctcctcgcccaccagcgtcctcctcgcccacggcgccgcgcgtgccacgtgagtagcgcgccgccgcgggccgaccgatggtgccgtggtctactggtcttgggcctcggaatcacgaggggcaaa

SEQ ID NO: 32 - DNA, 1725 bp, Zea mays
  source 1..1725: mol_type = other DNA
gccgcccagcgcccacccacacccctgcctcccaggctcccactacctgtccccaacaatgcacaccaacatccactctctcgtcaggctcctctcccccaaaaatgagcgctgagttctgcgctgctgccggtgtcgtggccatggagctcggagtcggagatgtgctggggctgcagcaaggcatcgcaatcaccgcgccatcgcccagggacagcgacctgggtcttctcaagcgagcaggcctcacccaggctgcggctgctgccccctacccctcccccttccttgacgaggagaagatgctcaggttctccaaggcggctcacacattgcactcaggcttggattttggaggcccaggtgagcaggctttcctgctgtccaggaccaagatgccatttactccctcgcagtggatggagctggagcaccaggctctgatatacaagtacctcaatgcaaaggcccccccataccttccagcctgctcatttcaatcagcaagagcttcagatcatccaatagagagcttgacagacttctctaacgcagtgagctggaggcctctgtatcaaggctacacaaatgcagactctgacccagaacctgggagatgccgacgaacggatggaaagaagtggcggtgctccaaggaagcaatggctgatcacaagtactgtgagcggcacatcaacagaaaccgtcaccgttcaagaaagcctgtggaaaatcaacctaagaagaccaccaaggaggtgcctgctgctgctggctcattgccatgtgctgggccacaaggtagcttgaagaaggcaaaagttaatgactccaagccaggcactgtcagctattgggcagatagtttaaacaggacaatgttgagcagagagaaagcaaacaaaccgacggaagatagctctttgctgcttacttctacgaacagccaacccaccttgtccctgctctctcagctgaagcagcaaaacaaaccagataagttaggccccacactggaaaatgagtcaaacccagacacaatattgaaagcctggggtggcaaccagcctagccacaagagcatttcctctacagagcgccatgatgctgaatccctccaatcagtccttcaaaatttcagcctagcccagaatgagaagatggagtcagaaaagaacaaatattctgattccgtgctagtttcgtcgactttctattctgcagacggtccaagagctacctgccttacacctaacatgacacaggtgaagcaggattgcatatcaagctcttgggagatgcctcaaggtggacctctaggcgaaatcttaacgaactccaagaatagcaaggacttaagcaagtgtaaaccaaggtcatatggttggttgttgaatcttgaccatgcaccatgattcctcaatccatgaagagcttgacatagatatcccatcatgtaggcaaacaatggtcagaaaaaggttatgaccacattgcttgccccatgcatgcttgctatctacatttgtatttctgttacgtagcatttagctagttgaattatcagttcttctggatacggctgtatcttaaacaagttctagtttgtgtcagatgatatcttgcttgctagatgtttcatgttccactttcaacaggaacttcagacaaccactttgattgacagaaaactgtttgaagaaccagctctagtaaacaaggttc

SEQ ID NO: 33 - DNA, 1668 bp, Zea mays
  source 1..1668: mol_type = other DNA
aaatccaaagcgagcagatcccccgcccctaaccgactcccctcgcttccattccattaatggccgccaccaagagccctccaacacacacctgacctccccttcccccctctctccgccgcccgttccccgcgcctccgcccgtacgtcccgttcccggtcggccggccggtccaaagggaggggaggaggaggaacgcgggagtcggggcccgcaccgatgctgagctcggcatcctcggccgcgggagcggccatggggatgggcggcggcgggtacgcgcaccagcccccgccacagcgcacggtcttcaccgccgcgcagtgggcggagctggagcagcaggcgctcatctacaagtacctcatggccggcgtccccgtcccgcccgacctcctcctccccgtccgccccggccccgccgccgccttctccttcgccggccccgccgccgcgtcgcccttctaccaccaacaccacccgtccctgagctactacgcctactacggcaagaaactggacccggagccgtggcggtgccgccgcaccgacggcaagaagtggcggtgctccaaggaggcgcaccccgactccaagtactgcgagcgccacatgcaccgtggccgcaaccgttcaagaaagcctgtggaatccaagaccgcctcgtcgtcgtcgcccgcgcacccgtcgccgccccagctgtccaccgtcaccaccaccgcgcctctcgagccccttgcagcggcggggggcaaggtccacggcctgtccctcggcggcggcgctgctggctcgtcgcacctcggcgtcgatgcttcgaatgctcactatcgttatggtagcaacaggtaccctctcggagctaaaccggacggcggcgagttgagcttcttctcaggagcgtcatcggggaacaactcgaggggcggcttcaccatcgactctccatcagataacaactcgtggcactccgccctggcgtccagcgtgcccccgttcacgctgtcgacgaagagcggggactccggcctcctgcccggcgcctacgcctcctactcccagtcccactcccacatggagccgccgcgggagctcgggcaggtcaccatcgcctcgctggcgcaggagcaggagcgccagcagccgttcagtggtgggatgctcgggaacgtgaagcaggagaaccagaaccagccgctgcggcccttcttcgacgagtggcccgggacgcgggcggactcgtggccgccggagatggacggcgcgccgcgggccggcaggacctccttctcctcctccaccacccagctctccatctccatcccgatgcccagatgcgactgacgaggaaccgtcgatcgggcggccactagacggtggacgctcacgctcactagtgcgctgtcgcctggagtggagatcgatcgtcacgccgccgccgcctacgccctcgtcctacctatttgttttgccgtagtgtattgtaccgacctcattaagaacagaacagaacagaagtctctgtgtaagagccggctccttcctgttcctatggtttgtactagtagcgtgctttgtatttgttgtacccctcaagaccgagaatgaaacccgcccgccgtcagtctgtctttgcgttttagtagatgatgcgagtgtttcgtgtg

SEQ ID NO: 34 - DNA, 27 bp, synthetic construct
  misc_feature 1..27: note = Synthetic binding site of GRF5
  source 1..27: mol_type = other DNA
aaccgttcaagaaagcctgtaaaaacg

SEQ ID NO: 35 - DNA, 27 bp, synthetic construct
  misc_feature 1..27: note = Synthetic binding site of GRF6
  source 1..27: mol_type = other DNA
aaccgttcaagaaagcctataaaaacg

SEQ ID NO: 36 - DNA, 27 bp, synthetic construct
  misc_feature 1..27: note = Synthetic binding site of GRF6
  source 1..27: mol_type = other DNA
aaccgttcaagaaagcctgtcaaaacg

SEQ ID NO: 37 - DNA, 27 bp, synthetic construct
  misc_feature 1..27: note = Synthetic binding site of GRF6
  source 1..27: mol_type = other DNA
aaccgttcaaaaaagcctataaaaacg

SEQ ID NO: 38 - DNA, 27 bp, synthetic construct
  misc_feature 1..27: note = Synthetic binding site of GRF6
  source 1..27: mol_type = other DNA
aaccgttcaagaaagcctctcaaaacg

SEQ ID NO: 39 - DNA, 27 bp, synthetic construct
  misc_feature 1..27: note = Synthetic binding site of GRF6
  source 1..27: mol_type = other DNA
aaccgttcaagaaagcctgtacaaacg

SEQ ID NO: 40 - DNA, 27 bp, synthetic construct
  misc_feature 1..27: note = Synthetic binding site of GRF6
  source 1..27: mol_type = other DNA
aaccgttcaaaaaagcctgtggaaacg

SEQ ID NO: 41 - DNA, 27 bp, synthetic construct
  misc_feature 1..27: note = Synthetic binding site of GRF6
  source 1..27: mol_type = other DNA
aaccgttcaaaaaagcctgtaaaaacg

SEQ ID NO: 42 - protein, 9 aa, Zea mays
NRSRKPVET

SEQ ID NO: 43 - protein, 9 aa, synthetic construct
  REGION 1..9: note = Mutant GRF miRNA binding site
NRSRKPVKT

SEQ ID NO: 44 - protein, 9 aa, synthetic construct
  REGION 1..9: note = Mutant GRF miRNA binding site
NRSRKPIKT

SEQ ID NO: 45 - protein, 9 aa, synthetic construct
  REGION 1..9: note = Mutant GRF miRNA binding site
NRSKKPIKT

SEQ ID NO: 46 - protein, 9 aa, synthetic construct
  REGION 1..9: note = Mutant GRF miRNA binding site
NRSRKPVQT

SEQ ID NO: 47 - protein, 9 aa, synthetic construct
  REGION 1..9: note = Mutant GRF miRNA binding site
NRSKKPVKT

SEQ ID NO: 48 - DNA, 20 bp, synthetic construct
  misc_feature 1..20: note = Synthetic spacer
  source 1..20: mol_type = other DNA
caaccgttcaagaaagcctg

SEQ ID NO: 49 - DNA, 20 bp, synthetic construct
  misc_feature 1..20: note = Synthetic spacer
  source 1..20: mol_type = other DNA
aaccgttcaagaaagcctgt

SEQ ID NO: 50 - DNA, 20 bp, synthetic construct
  misc_feature 1..20: note = Synthetic spacer
  source 1..20: mol_type = other DNA
cgcaaccgttcaagaaagcc

SEQ ID NO: 51 - DNA, 20 bp, synthetic construct
  misc_feature 1..20: note = Synthetic spacer
  source 1..20: mol_type = other DNA
ctttcttgaacggttgcggc

SEQ ID NO: 52 - DNA, 20 bp, synthetic construct
  misc_feature 1..20: note = Synthetic spacer
  source 1..20: mol_type = other DNA
ttcttgaacggttgcggccg

SEQ ID NO: 53 - DNA, 20 bp, synthetic construct
  misc_feature 1..20: note = Synthetic spacer
  source 1..20: mol_type = other DNA
tcttgaacggttgcggccgc

SEQ ID NO: 54 - DNA, 20 bp, synthetic construct
  misc_feature 1..20: note = Synthetic spacer
  source 1..20: mol_type = other DNA
ttgaacggttgcggccgcgg

SEQ ID NO: 55 - DNA, 23 bp, synthetic construct
  misc_feature 1..23: note = Synthetic spacer
  source 1..23: mol_type = other DNA
cacaggctttcttgaacggttgc

SEQ ID NO: 56 - DNA, 20 bp, synthetic construct
  misc_feature 1..20: note = Synthetic spacer
  source 1..20: mol_type = other DNA
ttgaacggttgcggccgcgg

SEQ ID NO: 57 - DNA, 20 bp, synthetic construct
  misc_feature 1..20: note = Synthetic spacer
  source 1..20: mol_type = other DNA
cacaggctttcttgaacggt

SEQ ID NO: 58 - DNA, 20 bp, synthetic construct
  misc_feature 1..20: note = Synthetic spacer
  source 1..20: mol_type = other DNA
tttccacaggctttcttgaa

SEQ ID NO: 59 - DNA, 20 bp, synthetic construct
  misc_feature 1..20: note = Synthetic spacer
  source 1..20: mol_type = other DNA
tgcgtttccacaggctttct

SEQ ID NO: 60 - DNA, 20 bp, synthetic construct
  misc_feature 1..20: note = Synthetic spacer
  source 1..20: mol_type = other DNA
ttcaagaaagcctgtggaaa

SEQ ID NO: 61 - DNA, 20 bp, synthetic construct
  misc_feature 1..20: note = Synthetic spacer
  source 1..20: mol_type = other DNA
aacggttgcggccgcggtgc

SEQ ID NO: 62 - DNA, 20 bp, synthetic construct
  misc_feature 1..20: note = Synthetic spacer
  source 1..20: mol_type = other DNA
aagaaagcctgtggaaacgc

SEQ ID NO: 63 - DNA, 20 bp, synthetic construct
  misc_feature 1..20: note = Synthetic spacer
  source 1..20: mol_type = other DNA
tgaacggttgcggccgcggt

SEQ ID NO: 64 - DNA, 20 bp, synthetic construct
  misc_feature 1..20: note = Synthetic spacer
  source 1..20: mol_type = other DNA
ttgaacggttgcggccgcgg

SEQ ID NO: 65 - DNA, 20 bp, synthetic construct
  misc_feature 1..20: note = Synthetic spacer
  source 1..20: mol_type = other DNA
acaggctttcttgaacggtt

SEQ ID NO: 66 - DNA, 20 bp, synthetic construct
  misc_feature 1..20: note = Synthetic spacer
  source 1..20: mol_type = other DNA
cacaggctttcttgaacggt

SEQ ID NO: 67 - DNA, 20 bp, synthetic construct
  misc_feature 1..20: note = Synthetic spacer
  source 1..20: mol_type = other DNA
agaaagcctgtggaaacgca

SEQ ID NO: 68 - DNA, 20 bp, synthetic construct
  misc_feature 1..20: note = Synthetic spacer
  source 1..20: mol_type = other DNA
gaaacgcagctcgcgcccca

SEQ ID NO: 69 - DNA, 20 bp, synthetic construct
  misc_feature 1..20: note = Synthetic spacer
  source 1..20: mol_type = other DNA
caggctttcttgaacggttg

SEQ ID NO: 70 - DNA, 20 bp, synthetic construct
  misc_feature 1..20: note = Synthetic spacer
  source 1..20: mol_type = other DNA
acaggctttcttgaacggtt

SEQ ID NO: 71 - DNA, 20 bp, synthetic construct
  misc_feature 1..20: note = Synthetic spacer
  source 1..20: mol_type = other DNA
agaaagcctgtggaaacgca

SEQ ID NO: 72 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 73 - protein, 166 aa, synthetic construct
  REGION 1..166: note = Synthetic tRNA-specific adenosine deaminase Tad
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 74 - protein, 166 aa, synthetic construct
  REGION 1..166: note = Synthetic tRNA-specific adenosine deaminase Tad
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 75 - protein, 166 aa, synthetic construct
  REGION 1..166: note = Synthetic tRNA-specific adenosine deaminase Tad
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 76 - protein, 166 aa, synthetic construct
  REGION 1..166: note = Synthetic tRNA-specific adenosine deaminase Tad
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 77 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 78 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 79 - DNA, 621 bp, Petromyzon marinus
  source 1..621: mol_type = other DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 80 - protein, 160 aa, synthetic construct
  REGION 1..160: note = Synthetic polypeptide
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 81 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 82 - DNA, 1592 bp, Medicago truncatula
  source 1..1592: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 83 - DNA, 2000 bp, Zea mays
  source 1..2000: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 84 - RNA, 22 nt, Zea mays
  source 1..22: mol_type = other RNA
ttccacaggctttcttgaactg

SEQ ID NO: 85 - RNA, 21 nt, Zea mays
  source 1..21: mol_type = other RNA
ttccacagctttcttgaactt

SEQ ID NO: 86 - RNA, 21 nt, Zea mays
  source 1..21: mol_type = other RNA
gttcaagaaagctgtggaaga

SEQ ID NO: 87 - RNA, 21 nt, Zea mays
  source 1..21: mol_type = other RNA
gttcaataaagctgtgggaaa

SEQ ID NO: 88 - RNA, 21 nt, Zea mays
  source 1..21: mol_type = other RNA
ttccacagctttcttgaactg

SEQ ID NO: 89 - RNA, 21 nt, Zea mays
  source 1..21: mol_type = other RNA
ttccacagctttcttgaactt

SEQ ID NO: 90 - RNA, 21 nt, Zea mays
  source 1..21: mol_type = other RNA
gttcaataaagctgtgggaaa

SEQ ID NO: 91 - RNA, 21 nt, Zea mays
  source 1..21: mol_type = other RNA
tcccacagctttattgaactg

SEQ ID NO: 92 - RNA, 21 nt, Zea mays
  source 1..21: mol_type = other RNA
ggtcaagaaagctgtgggaag

SEQ ID NO: 93 - RNA, 21 nt, Zea mays
  source 1..21: mol_type = other RNA
tcccacagctttattgaactg

SEQ ID NO: 94 - RNA, 21 nt, Zea mays
  source 1..21: mol_type = other RNA
ggtcaagaaagccgtgggaag

SEQ ID NO: 95 - RNA, 21 nt, Zea mays
  source 1..21: mol_type = other RNA
ttccacagctttcttgaactg

SEQ ID NO: 96 - RNA, 22 nt, Zea mays
  source 1..22: mol_type = other RNA
ttccacaggctttcttgaactg

SEQ ID NO: 97 - DNA, 957 bp, Glycine max
  source 1..957: mol_type = other DNA
atgaataagagtgggggaggagagatgagtaagtggccgttcacaatgtctcagtggcaggaactggaacatcaagctttgatttacaaatacatggtggctggtcttcctgtccctcctgatctagtccttcccattcagaacagcttccactccatttcccaaaccttcttgcaccatccctctaccaccatgagttattgttccttctatgggaagaaggtggacccggagccaggacgatgcaggaggactgatgggaaaaagtggaggtgctccaaggaagcctacccagactccaagtactgtgagcgacacatgcaccgtggccgcaaccgttcaagaaagcctgtggaatcacaaactatgactcagtcatcatctaatgtgtcatcattgactgtaactgctggcagcagcagcagtgcaactggaaatttccataaccttaccaccacaaatgcatatggtaatccccaagggactggttctggatcagaccaaacccactatcacatggattccattccctatgggatcccaagtaaagaatacaggtattttcaaggacctaaatctgagggaggtgaacatagtttcttgtccaaaactttaggaagcaacaaggttcttcacatggagtcacagatggataacactttgatgccaaccagtggagttgcctcattctctacattgagatcaaataataattccatgttgcagggtgattatctgcagccttctttcttatctagtgagtatgcctcagcagaaactgtgaagcaagagggtcagttccttcgacctttctttgatgaatggcctaaaaggagggactcatggtctggtctggaagatgagggatccaaccacactcaactctcaatatccattcctatgtcagcatcaaatttctctacaactagctctcattccccacatggtgagatttaa

SEQ ID NO: 98 - DNA, 1377 bp, Glycine max
  source 1..1377: mol_type = other DNA
atggaagctaagcctcttcgaactgttccctcttcacacaacacttctggaggtggaccccagaagaagattgacatgggacacaagatggtaggtgatgttggtgttgttgttgttgatgaaagaaagagggttgttgtggtcaaggaagagaaagagaacacttcttcctacagtgttgaacttcatctcggggttgatgcttctcattctgctccacaagaaataaaccatgtgatcactgaagctcagaggcgtgagcttcaccatcaagttttcatcttcaaccatttggcatataatcttcctcctccttatcaccttgtgcaattcccgagcaacatgtcagaatacagtttcctgggttttgatcatgggagtatggtggatccagagcctcataggtgtagaagaactgatggaaagaaatggaggtgtggtaagaatgtggtgcctaaccagaagtactgtgaaagacacatgcatagaggtcgaaatcgttcaagaaagcctgtggaaacatctcaaattaactctcatctggcaacaaagccatctagcaagtcacacaacaaacccgcctcaaggacacaatttgaaatttcaaatccaaaccttatggccattcgacatagtgacacatcaagtaccccatcaaggagtctcagtgttgccaattgctcttctgctaataataggtggaaaaatagtgcaagttatgctgactacctcacatcgttttcttctgcgtctgcagtgtcccctggatccactcttgccactccagttgctcctaagatggccaccttcagcagtgtgacatccattgcttcagatagaggaagttgcctaaatatatgccagaaagataacaagtccaagagctgtatcagcaacaacatcagtgttaaaagtggtgggaaaggaagaattgttggtgacactaatggcatttctactggaataggcttctccccaacgagtgttcttcaagtttctggttgcaacccttcatacctgaatgacagaaccaacatagaatctgcatccggtaggtgccggagaacagatggtaagaagtggcaatgcaagagtgctgttcttccgggtcagaagtactgtgccacacacatgcacagaggtgctaaaaagcggctcacaagccatgaaccggcagttaccattgcgcggttgcctaactcttcagtcacaaccaaaatgcagaaagcgcattgtgcaattccaaacacaaatctttccatgtcagtcccagcaagtgaaccattcatacaatgtaatgagaaaagtcaaagtaccagtgacactgataccaccatcagtgacaccttgaatgagtgtagctatgcttctttctga

SEQ ID NO: 99 - DNA, 1008 bp, Glycine max
  source 1..1008: mol_type = other DNA
atgagcgttcctccgccgtatgcggcggcgatgtggacgccgttcacggcggcacagtggcacgagctggagcaccaggccctcatcttcaagtacctcaaggcgggtctctcggttccccctgacctccttctccccattcgcaagagcctccagttgatgtctcatccatctctggggttttatgggaagaagatagaccctgaaccgggtcggtgcaggagaaccgatgggaaaaagtggaggtgttcgagggatgcgcaccctgactccaagtactgcgaccgccatatgatacggcggcgttaccgttcaagaaagcctgtggaatcatctcaaactcactcttcttcttcttcttcttcttcttcttctgctactgctgcttcttcaaattcaaatcctgttgctggtggtggtggtggttctgttgctaatgtttctggaactgcaactgctgcaaaaacccttcacacccttcccttgcacaccaatggtgcaagggaaggtttcactttcacccttgggaacaacaccaacaccaccctcccacaccttcacatgaaccccctcactctttcagctgacaataccaagaaaacttacaggtttggactgaactcagaagcagatgagcacaacgtcttacagaaagatctgggaagtgtgaggtatcaaggttatgactttacctcagatgccatgtggtctcaaatgtctcacattccatcaaatactgtttcggaatcaagaagtggttctactatgctgggcaactgcttccaacatcaaacaatgcgagatgctgagctgttgaacctggaaacggcaaggaccaaagatcttgtctttagtggccagttaagttcagctgggggtttgaaacaggaatatcagtctccccagtctctcttcagtgactgggactggaagaaggatttaagttcctctgcccttgagtataagcctaacaaggattttaactgcaacccagatgccaatgtttga

SEQ ID NO: 100 - DNA, 1155 bp, Glycine max
  source 1..1155: mol_type = other DNA
atgaatggaaggaacgttaacacaaacaggttccctttcactccttcccagtggcaagagcttgaacaccaagctctcatctacaaatacatggcttcaggtatctccatcccccctgaccttctcttcaccatcaaaagaacaacccacttggactcctcaagactattgcctcaccaccctcaacactttggatggaactatttgccaatggggttggggaggaaaatagacccggagccagggaggtgtagaagaacagatggaaagaaatggaggtgctcaaaggaggcgtatccagattcaaagtactgtgagaggcacatgcacagagggaaaaatcgttcaagaaagcctgtggaagttttgaaaacaacaccaacgacagcagcagtggcaacaaacacagatgcctcaaccccaacaacaatcttatcaatcaccaaaaacagtcctgcacatgcactctccccaaccactcattctctctctcatgacacttaccatcatcatcatcatcaccctcaccctcagcaacattcctcccactccttcctctatcatcattcttcgaggccctcttccggtgctgctttgtcatttcaagacaacagtgcccccttgtttctcgacactgcttcttgctctcagaataacaacaacactgactgcaggaacaggtatgtgtatggactgaaggaggaggtggacgagcatgccttcttcacagaaccttctggaactatgagaagcttctctgcttcctcaatggaagattcatggcaactcacaccactgactataagctcctcttcctcttcgaaacagaggaactgctctggtttatccaatgacaacaacgagtactcctacttgcaacttcagagcctcaatggcaacaactcaaaacagccacaacaagatcaaggttgctacatatcaggcagtgatgtcaagtgcgaaacattcatgaaacttgggaaagaagaacctcagaaaaccgttcatcgcttcttcgatgaatggccccctaaaagcagaggatcgtggcttgatttggatgataaatcatccaccacccagctttcaatttccattccaacttctactcatgattttgcaactttcaattcaacaacccaacgagatggttga

SEQ ID NO: 101 - DNA, 1098 bp, Glycine max
  source 1..1098: mol_type = other DNA
atgatgaatggaagaaacaggtttccctttaccccatcacagtggcaagagcttgaacaccaagctctcatctacaagtacatggcttcaggcatttccattccccctgatcttctcttcaccataaaaaggagctattttgattcccctctctcctcaaggcttttgcctaaccagccacaacactttggatggaactaccttcagatgggtttgggaagaaaaatagaccctgagccaggtaggtgtagaagaactgatggcaagaaatggagatgctcaaaagaagcatacccagattctaagtactgtgagaggcacatgcacagagggaagaatcgttcaagaaagcctgtggaagttttgaaaacaacaacaccatcatcaacaatgacaacaacaaacacaaactcaaatgcttcttcaacacaacaagcaatctcatcaatcaccaaaattaatactctttcacctcttgcttcatctgagactcaccaacaccaccctcaacactatggctcctttctctatcatcatcaccctccttcaaggtcctctggcattggcttgtcctttgaagataatagggctcccttgtttcttgacactggctcttgctctcagtccaacacagactgcaggagtaggtatgtttatggagagaaagaggaggtggatgagcatgctttcttcacagaaccttgtggtgttatgaaaagcttctctgcttcctctatggatgactcatggcaactcacaccattgactatgagttcctcatcttcttcttccaaacagaggagttcctttggcttgtccagtgattactcttgcttgcaacttcagagccactcaaagcagcagcaacaagagcatcatcaagatcagggttgctacatgtttggtggtggtcaagttgtgaaagaagaacctcagaaaacggttcatcgcttctttgatgaatggccccacaaaggaagagaagggtcttggcttgatttggatgacaaatcttccacaacccaactttcaatttccatccccacatgttctcatgattttccaactttcagttctagaaaccaccacgatggttga

SEQ ID NO: 102 - DNA, 1014 bp, Glycine max
  source 1..1014: mol_type = other DNA
atgagcgttcctccgccgtctacggcggcgatgtggacgccgttcacggcagcgcagtggcacgagctggagcaccaggccctcatcttcaagtacctcaaggcgggtctctcggttccccctgacctccttctccccattcgcaagagcctccaattgatatcgtctcacccatcgatggggtattatgggaagaagatagaccctgaaccgggtcggtgcaggagaaccgatgggaaaaagtggaggtgttcgagggatgcacaccctgactccaagtactgcgaccgccatatgatacgacgtcgttaccgttcaagaaagcctgtggaatcatctcaaactcactcttcttcttctgcttctgctgcttcttcaaattcaaaccctgttgctgctggtggtggtggtggttctgttgctaatgtttctgcaactgctgcaaaaaaccttcacacacttcccttgcacatcaatggcgcaagggaaggtttcactttcacccttgggaacaacaccagcaccaacaccaacaccaacaccaacaccctccctcaccttcacatgaaccccctcactctttcagctgacaataccaagaaaacttacaggtttggactgaactcagaagcagatgagcacaacgtcttacagaaagatctgggaagtgtgaggtatcaaggttatgactttacctcagatgccatgtggtctcaaatgtctcacattccatcaaatactgtttcggaatcgagaatcggttctactatgctgggcaactgcttccagcatcaaacaatgcgagatgctgagctgttgaatctggaaacggcgaggaccaaagaccttgtctttagtggccagctaagttctgcagggggtatgaaacaggaatatcagtctccccagtctctcttcagtgactgggactggaagaaggatttaagttcctctgcccttgagtataggcccaacaaggattttaactgcaacccagatgtcaatgttgattaa

SEQ ID NO: 103 - DNA, 1803 bp, Glycine max
  source 1..1803: mol_type = other DNA
atggatcttggggtggtgggtttggagggggtggtgggttcagaaagtggttgtgtgtttggttcttctcttgtttcagatcctgagacaaagcacaagtggtacggatctggtttgctcaagcaagagagatctgccatagctactgaagatgatgagtggagaatttccaaagttgctaaaactgatcatgacatgtcttcagcctccaaagcaatgctctttcagcaaagaaacaactctttgttgagatctaataatgcaactctcttctctgatggtcatcaccaatcacaaatgttgagcttctcttctccaaagtcagattctttgttgatagataaggcttcctcaaatgccacattgcctttttcttcccaccaattgtctagctacaccagaaatacaggttacaattcaggaagcataagcatgcatggggctttggctagtgtgagagggccattcactccatcacagtggatggagcttgaacaccaagccttgatctacaagtacatcacagcaaatgttcctgtgccaactcatcttctcattcccatcagaaaagcacttgattctgttggcttctgcaacttctcagccggactcctcagacccaactcattgggatggggaggtttccatctaggattctcgaacaatacagaccctgagccagggaggtgtaggagaacagatggaaagaaatggcgatgttcaagagatgccgtagtagatcagaagtattgcgagcggcacatgaaccgaggacgccatcgttcaagaaagcctgtggaaggccaatcaggccatgccctcaccaccaccaccagtaatacacctaatgcttcctccaactctgtggtgcctggcaacaacaacaacacctttgcacacaacaatgtgcaccaccctattcctcctcattcctctccggtcaacaccatcactaggatgtttacaagcaacaaagagaataataacagtaccagtgagaggatgcaggaccctgcacttcccatgcttcctcccactcttgagctgaaaccaaaggagaacaatcctttcatgattcataaacaccaaatcccatctgatgaatactcaagtaggaacaacaatgagtttgggcttgtcacttctgattctttgcttaacccctcagagaaaagaagctttacttcttcacaaaagaatgattcttctgagtcccaacaacaacattccctcaggcacttcattgatgactctcccaaaccacagtctaatcatcatcatcgttcgtcgtctatatggcctgaacttgacaacatgcagtcagacaggactcagttatcaatctccataccaatatcttcctcagatcacttcatgtcattcactacttccttgccctcgaacgagaaactcacgttgtcaccacttaggctttcaagggagttagaccccattcaaatggggttgggagtgggaagtgcccccaatgaagcaaacactaggcaagccaattggattccaatcacttgggagagttcaatgggtggtcctcttggagaggttttgaaccttagtaacaataacaacagcaatgctagtgatcaatgtggcaagaacaacaacaacacttcagctctcaacctcatgaaagatggatgggacaataatcctccatcagggtcatccccaactggggtgcttcaaaaatctgcatttggatcactttccaatagcagtgctgggagcagtccaaggggggcagagaacaacaaagaaggtgccaccttgtgcaatgccttgtaa

SEQ ID NO: 104 - DNA, 1134 bp, Glycine max
  source 1..1134: mol_type = other DNA
atgatgaatggaaggaacgttgacacaaacaggttccccttcactccttcccagtggcaagagcttgaacaccaagccctcatctacaaatacatggcttcaggtatttccatcccccctgaccttctcttcaccatcaaaagaacaacccacttggactcctcaagactcttgcctcaacactttgggtggaattatttgccaatgggattggggagaaaaatagacccagagccagggaggtgtagaagaacagatgggaaaaaatggaggtgctcaaaggaggcgtatccagattcaaagtactgtgagaggcacatgcacagagggaaaaaccgttcaagaaagcctgtggaagttttgaaaacaacaccaatgacagcagcaacaaacacagatgcctcaaccccaacaacaatcttatcaatcaccaaaaacagtcctgtactcttcccaaccactcattctctgtctcatgacacttaccatcatcatcaccatcatcactctcaccctcagcaacattcctcccactccttcctctatcattcttcgaggccctcttccggtggtgtttctttgtcatttcaagacaacagtgcccccttgtttctcgacactgcttcatgctctcagaataacaacaacactgactgcaggtatgtgtatggactaaaagaggaggtggatgagcatgcctttttcacagaaccttctggaactatgagaagcttctctgcttcctcaatggaagattcatggcaactcacaccactgactataagctcctcttcctcttcaaaacagaggagttgctctggtttatccaatgacagcaatgagtactcctacttgcaacttcagagcctcagtggcaacaactcaaagcatccacagcaagatcaaggttgctacatatcaggcagtgatatcaagtgcgaaacattcatgaaacttgggaaagaagaacctcagaaaaccgttcatcgcttcttcgatgaatggcctcccaaaagcagaggatcatggcttgatttggatgataaatcatccacaacccagctttcaatttccattcccacttctactcatgattttgcaactttcagttcaacaacccaacgagatggttga

SEQ ID NO: 105 - DNA, 1830 bp, Glycine max
  source 1..1830: mol_type = other DNA
atggatcttggggtggtgggtttggagggggtggtgggttcagaaagtggttgtgtgtttggttcttctcttgcttcagatcctgagacaaagcacaagtggtacggatctggtttgctcaagcaagagagatctgccatagctactgaagatgatgagtggagaatttccaaagttgctaaaactgatcatgacatgtcttcagcctccaaagcaatgttctttcagcaaagaaacaactctttgttgagatctaataatgcaactcttttctctgatggtcatcaccaatcacaaatgttgaacttctcttctccaaagtcagagactttattggtagataaggctttctcaaatgccacattgcctttttcttatcatcaattgtctagttacagcagaaatacaggttacaattcgggaagcataagcatgcatggggctttggctagtgtgagagggccattcactccatcacagtggatggagcttgaacaccaagccttgatctacaagtacatcacagcaaatgttcctgtgccaactcatcttctcattcccatcagaaaagcacttgattccgttggcttctgcaacttctcatctggactcctcagatccaactcgttgggatggggaggcttccatttgggattctccaacagcacagaccctgagccagggaggtgtaggagaacagatgggaaaaaatggcgatgctcaagagatgctgtagtagatcagaagtattgcgaacggcacatgaaccgaggacgccatcgttcaagaaagcctgtggaaggccaatcaggccatgccctcaccaccactaacacacctaatgcttcctcaaactcaaccgtggtgcccggcaaccacaacaacaacacctttgcacacatcaatgtgcaccaccctcttcctcctcattcctctccagccaataccatcaataggatgtttatgagtaataataaagagaacaacaacacgagtgagaggatgaaggatggccttgcccttcccatgctacctcctactcttgagctgaaaccaaaggacaacaacaactctttcatggttcataaacaccaagaaccatatgatgaatcctcaaggaacaagaatgagtttggactcgtcacttctgattccttgcttaacccttcacagaaaagaagctttgattcttcttcttcttcttcacaaaaggatgattcttctgagtcccaacaacaacattccctcaggcacttcattgatgactctcccaaaccacagtctcatcataatcataaccatcgttcgtcgtcatctatttggcctgaactcgacaacatgcagtcagacaggactcagttatcaatctccataccaatatcttcctcagatcacttcatgtcatttgcaacttcctcgccctcgaatgaaaaactcacattgtcgccactaaggctttcgagggagttcgaccccattcaaatgggattaggagtgggaagtgcctccaatgaagcaaacactaggcaagccaattggattccaatcacttgggagagttcaatgggtggtcctcttggagaggttttgaaccttagtaacaataataataacagcaatgcaagtgatcaatgtggcaagaacaacaacaacacttcagctctcaacctcatgaaagatgatgacgatgatgatgggtgggacaatagtcctcctccaatagggtcatcaccaactggggtgcttcaaaaaacagcatttggatcactttccaacagcagtgctgggagcagtccaagaggggcaccagagaacaacaacaaagaatag

SEQ ID NO: 106 - DNA, 1800 bp, Glycine max
  source 1..1800: mol_type = other DNA
atggatcttggggtggtgggtttggagggggtggtgggttcagaaagttgttgtgtgtttggttcctctcttgcttcagatcctgagacaaagcacaaatggtacggatctggtgtgctcaagcaagagagatctgccatagcaagtgaagatgatgagtggagaacttctaaagtggccaaaactgatcatgacatgtcttctgcctccaaagcaatgctctttcagcaaagaaacaactctttgttgagatctaatgctacccttttctctgatggccatcatcaccaatcacaaatgttgagcttctcttctccaaagtcagagtctttgttggtagataaggcttcctcaaatgccacattgcctttttcttaccaccaattgtctagcaacaccagaaatgcaggttacagttcaggaagcataagcatgcatggggctttggctggtgtgagagggccattcactccgtcacagtggatggagcttgaacaccaagccttgatctacaaatacatcacttcaaacgtgcctgtgccaacccatctcatacccattagaaaagcacttgattctttgggcttctgcaacttttcaactggactcctaaggcccaacgcattgggatggggaggtttccatctgggattctcgaacaacacagaccctgagccaggcaggtgtaggagaacagatggaaaaaaatggcgatgctcgagagatgccgtagtggatcaaaagtattgcgagcggcacatgaatagaggacgccatcgttcaagaaagcctgtggaaggccaattaggccatgccctcaccaccaccaccaccaccaatacacctaatgcttcctccaactctaccgtggtgccccgcaacaccaccaccaccaccacctttgcacacaacaatgtgcaccaccctcttccacctcattcgtctcaggccaacaccatcaataggatgtttacaagcaacaaagagaataacagtaccagtgagaggctgcaggaccctgctcttcccatgctgcctcccactcttgagctgaaaccaaaggagaacaatcctttcatgattcataaacaccacatcccagctgatgaatactcaaggaacagcaatgagtttggactcgtcacttctgattccttgcttaaccctacacagaaaagaagctttacttcttcacaaaaggatgattctgagtcccaacaacaacattccctcaggcacttcattgatgactctcccaaaccacagtctcataatcatcaccaccatcatcattcgtcgtctatttggcccgaactggacagcatgcaatcagacaggactcagttatcaatctccataccgatatcttcctcagattttatgtcattcactacttcctcaccctccaacgagaaactcacgttgtcaccattggggcattcgagggagttagaccccattcaaatggagttgggaatgggaagtggtgcctccaatgaagcaaacaccaggcaagccaattggattccaatcacttgggagagttcaatgggtggtcctcttggagaggtattgaaccttagcaataacagcaatgcaagtgatcaatgtggcaagaacaacaacacttcagctctcaacctcatgaaagatgggtgggacaataatcctccactagggtcatccccaactggggtgcttcaaaaatcagcatttggatcactttccaatagcagtgctgggagcagtccaagggcagaaaacaacaaagaaggtgccaccttgtgcaatgccttgtaa

SEQ ID NO: 107 - DNA, 1785 bp, Glycine max
  source 1..1785: mol_type = other DNA
atggatcttggggtagtgggtttggagggggtggtgggttcagaaagtggttgcgtgtttggttcctctcttgcttcagatcctgagacaaagcacaagtggtacggatctggtgtgctcaagcaagagagatctgccatagcaagtgaagatgatgagtggggaacttataaagtggccaaaattgatcatgacatgtcttctgcccctaaagcaatgatctttcagcaaagaaacaactctttgttgagatctaataatgctactcttttttctgacggccatcaccaatctcaaatgttgagtttctcttctccaaagtcagagactttgttggtagatacagcttcctcaaatgccacattgcctttttcttaccatcaattgtctagttacagcagaaatacaggttacagttcaggaagcataagcatgcatggggctttggctggtgtgagagggccattcactccatcacagtggatggagcttgaacaccaagccttgatctacaaatacatcactgcaaacatgcctgtgccaactcatcttctcatacccatcagaaaagcacttgattctgttggcttctgcaacttctcaactggactcctcagatccaactcattgggatggggaggtttccatctgggattctcgaacagcacggaccctgagccagggaggtgcaggagaacagatggaaagaaatggcgatgctcaagagatgccgtagtagatcagaagtattgtgagcggcacatgaacagaggacgccatcgttcaagaaagcctgtggaaggccaatcaggccatgccctcaccaccaccaccaccactactaacacacttaatgcttcctcaaactcaaccgtggtgcccggcaacaacacctttgcacacaacaaagtgcaccaccctcttccacctaattcctctccagccaataccatcaataggatgtttatgaataataaagagaataacagcacgagtgagaggatggaggattgctctgcccttcccatgctacctcccactcttgagctgaaaccgaaggagaaaaacaatttcatgattcataaacaccaagacccctatgatgaatcctcaaggaacaacaatgagtttggactcgtcacttctgattccttgcttaaccctacacagaaaagaagctttgattcttcttcttcacaaaaggatgattctgagtcccaacaacaacattccctcaggcacttcattgatgactctcccaaaccaccatctcataatcatcatcgttcgtcgtccatttggcccgaactcgacaacatacagtcagataggactcagttatcaatctccataccaatatcttcctcagacttcatgtcattcactacttcctcaccctccaacgagaaactcacgttgtcgccacttaggctttcgagggcgttagaccccattcaaatggggttgggagtgggaagcggtgcctccaatgaagcaaatcctaggcaagccaattggattccaatcacttgggagagttcaatgggtggccctcttggagaggttttgaaccttagcaataacagcaatgcaagtgatcaatgtggcaagaacaacacttcagctctcaacctcatgaaagatgatgatgatgatgggtgggacaatagtcctccactagggtcatcaccaactggggtgttgcaaaaaacagcatttggatcactctccaacagcagtgctgggagcagtccaaggggggcaccagagaacaacaaagaatag

SEQ ID NO: 108 - DNA, 1002 bp, Glycine max
  source 1..1002: mol_type = other DNA
atgaacatcagtggcggaggaggaactgtgatgggtttcagtagtaatgggaggtccccattcacagtgtctcagtggcaggaactggagcaccaagctttgattttcaagtacatggttgcgggtcttcctgtgcctcccgatctcgtcctccccattcagaagagcttcgactctactctctctcacgctttctttcaccatcccacactgagttattgttccttctatgggaagaaggtggaccctgagccaggacgatgcaggaggactgatggaaaaaagtggaggtgctccaaggaagcatacccagactccaagtactgcgagcgccacatgcaccgtggccgcaaccgttcaagaaagcctgtggaatcacaaactatgactcactcgtcttcaactgtcacatcactcactgtgactggtggtggtgacagtaatggaactgtaaacttccaaaaccttcccacaaatgcctttggtaatctccagggtactgattctggaactgaccgcacgaattatcatctagattccattccctatgcgattccaagtaaagaatacaggtgtcttcaaggacttaaatctgagggtggtgaacactgcttcttttctgaagcttctggaagcaacaaggttctccaaatggagtcacagctggaaaacacatggccttcgatgtcaaccagagttgcctctttttctacatcaaaatcaagtactgattccctgttgcatagtgattatccccaacattcgtttttatctggtgaatatgcatcgggagaacacgtgaaggaggagggccagcctcttcgacctttttctaatgaatggcctaaaagcagggagtcatggtctggtctggaagatgatatatccaaccaaacagccttctccacaactcaactctcaatatccattcctatgtcttccgatttctctgcaacgagctctcagtccccacatggtgagaatgagattcaatttaggtaa

SEQ ID NO: 109 - DNA, 1737 bp, Glycine max
  source 1..1737: mol_type = other DNA
atgggtgaattatttggcgttggaaaaagaaggaacatcagcagcagcaacaacaacaacaacagttctagtagtagcgttcttgggttggatgtgaaggtgcaacagagccctgaagcattattccataataggatgatgatgatggcgcatcataatcatcaccaccgtccattgtcatcaccctttgataataatggtgatggcgatggtcccacgacttacatgtcttttactaatcatataaaccttgttagtggtgcttcttctgttcttggtcctgctattgatgctggctgtggtgctgctcctcctgcacctgtaagaactttgcagccttttgacatttcttcttatacttcttctcccaccaccactaccacaaccttcaacttcaaacccccttcagcaggtgtgatggcggcttcgttggggtttcctttcacaagtgcacaatggagggagcttgaaagacaagctatgatatacaagtacatgatggcttctgttcctgttccacatgatctcctcacaccctcttctcgctcttcctgcatggatggtggtttcaatctgaggttggcaaatagcactgaccctgagccaggtaggtgtagaagaacagatggtaaaaaatggagatgttcaagagatgtggctcctaaccacaagtactgtgagcgccatatgcatagaggccgtccccgttcaagaaagcctgtggaagttaacaccaacagcaccaccactcccactagcgtcaacaataacaaccatcaaatcaaaaaggctcgccatgagtgtaataataatccttttgctacacctgacgttactgcggctatttccaaccccacatccagaaaaaatggatcttctccccattttcttgggtctactaccactcagccataccttgattcttccctctcccttgataactttggtctaaaagctgctagttttgactccgtggcttctgtttctgctaataaggaacccaggggtttagagtggatgctgaatggagatcctatttccctgggtgcttctgactcacaatggcagtctctgatgcacaataaagatggaatgaccagtgttagttcctgtaacaccaccgagtctcagtatctgaattcattagcactatataactctggactagaacaacagaatagacgccatcctttgttcctgaaccctcttgttgttcccatggaaaatctccaaccggagaaaccaaggggttttattgatgcttggtctaacgctgaaagcaatgccaacaccaacaccaccaacaagaactctgctgcatcaattggtaaattatccctttcttctcttgatctatcaatggggggtgctgctgtgaatgaagatgtgggtaatgttaacatgggtttgggcctaatggagcctaatggaaaaacgcacactggtactaaaatttctctctccaattggcaaaacccagcaccttgggtggcttcatcacttgggggtccactagctgaagttctaaggtcaagcacagtcactgccaccaccaccaccaatgaagcaacctccaacacaccctcgccagccaccactacacatgctgaatctccatctggggtgttgcagaaaacgcttgtttcattgtctgatagcagtaacaatagcagcccaagggttgcatcatcaagggccaattctgagatggccttgctaaggtttcaatcaaattaa

SEQ ID NO: 110 - DNA, 927 bp, Glycine max
  source 1..927: mol_type = other DNA
atgagtaagtggcctttcacaatatctcagtggcaggaactggaacatcaagctttaatttacaaatacatggtggctggtcttcctgtgcctcctgatctagtcattcccattcagaacagcttccactccatttcccaaaccttcttgcaccatccctctaccaccatgagttattgttccttctatgggaagaaggtggacccggagccaggacgatgcaggaggactgatgggaaaaagtggaggtgctccaaggaagcctacccagactccaagtactgtgagcgacacatgcaccgtggccgcaaccgttcaagaaagcctgtggaatcacaaactatgacacagtcatcatccaatgtgtcatcattgactgtaactgctggcagcagcaccagtgcaactggaaatttccagaacctttccaccacaaatgcatatggtaatccccaagggactgcttctggaacagaccaaacccactatcacatggattccattccctatgggatcccaagtaaagaatacaggtattttcaaggatctaaatctgaggaacatagtttcttgtccaaaactttaggaagcaacagggttctacacatggagccacagatggacaacactttgatgccaaccggtggagttgcctcattctctacattgagatcaaataataattccatgttgcagggtgattatctgcagccttctttcttatctagtgaatatgcctcggcagaaactgtgaagcaagagggtcagtcccttcgaccgttctttgatgaatggcctaaaagcagggactcatggtctggtctggaagatgagagatccaatcacactcaactctcaatatccattcctatgtcatcgtcaaatttctctgcaactagctctcattccccacatggtgagatttaa

SEQ ID NO: 111 - DNA, 1008 bp, Glycine max
  source 1..1008: mol_type = other DNA
atggacttgcagttgaagcaatggagtaaccggcatgagtcagaacaagaacattattccccaaacatgccaaaatttctccctcaacaccacccaccaccatctccctctgcactccctctctttgtacctgaacaacccaacaccaaagtctgcaccctgtcagcattttctgattccacactaccttctgctcccagattttccaggatggagagttgcttcagctttgcacagtggcaagagcttgagttgcaggctctgatattcaggtacatgctggccggtgctcctgttcctcctgagctccttctaccaatcaagaaaagcttccttcaactttataaccctcctttgttggaatcagggtactactggggaagagcagcgctggatccggagccggggcggtgccggaggaccgacggcaagaagtggcggtgctcgaaggacgcggtggcgggtcagaagtactgcgaccgccacatgcatcgtggccgaaaccgttcaagaaagcctgtggaacaacgtgatggatctctttctgctatagcctccgtttcttcttcaccctcttcttcatttaatctccttcacctcagtgaaagttcctctggggccaagagtgacaacaagagcttctttgaaaaccatgatcatgtggatggggatggaaattcagccaaatctgatggccatgtcttgaggcatttctttgatgattggccaaggacactgcaagagcctgacaatggtgaaagcaatggttgccagaacaacaactcaggaacatgtctttctatgtcaacaccaggaatcacttcctcggatgtgtcgttgaaattgtccactggccatggagaggatgcgtgccacgcggcctcaatgggaggaccacttgcagaggcattaagatcatccaccaccagctccacttcttcaccaaccagtgttctgcttcagttgcctcctagttctgcttctgaggctagtttcattagcacctaa

SEQ ID NO: 112 - DNA, 1026 bp, Glycine max
  source 1..1026: mol_type = other DNA
atgaacaacagcagtggcggaggaggacgaggaactttgatgggtttgagtaatgggtattgtgggaggtcgccattcacagtgtctcagtggcaggaactggagcaccaagctttgatcttcaagtacatgcttgcgggtcttcctgttcctctcgatctcgtgttccccattcagaacagcttccactctactatctcgctctcgcacgctttctttcaccatcccacgttgagttactgttccttctatgggaagaaggtggaccctgagccaggacgatgcaggaggactgatggaaaaaagtggaggtgctccaaggaagcatacccagactccaagtactgcgagcgccacatgcaccgtggccgcaaccgttcaagaaagcctgtggaatcacaaactatgactcactcatcttcaactgtcacatcactcactgtcactgggggtagtggtgccagcaaaggaactgtaaatttccaaaacctttctacaaatacctttggtaatctccagggtaccgattctggaactgaccacaccaattatcatctagattccattccctatgcgattccaagtaaagaatacaggtatgttcaaggacttaaatctgagggtggtgagcactgctttttttctgaagcttctggaagcaacaaggttctccaaatggagtcacagctggaaaacacatggcctttgatgtcaaccagagttgcctctttttctacgtcaaaatcaagtaatgattccctgttgcatagtgattatccccggcattcgtttttatctggtgaatatgtgtcgggagaacacgtaaaggaggagggccagcctcttcgacctttttttaatgaatggcctaaaagcagggagtcatggtctggtctagaagatgagagatccaaccaaacagccttctccacaactcaactctcaatatccattcctatgtcttccaatttctctgcaacgagctctcagtccccacatggtgaagatgagattcaatttaggtaa

SEQ ID NO: 113 - DNA, 1155 bp, Glycine max
  source 1..1155: mol_type = other DNA
atggacttccatctgaagcaatggagaaaccagcacgagtcagaggaacaacattctacaaagatgccaaaacttctccctgaatcccatcaacaacaacagccatcagcctctgcactccctttgtttgtacctgaacccaacagcagcaaagtcagcaccctgtcagattcaacattagcttctgctacaatgacttctaccaccactaacagattatttcccaggatggggagctacttcagcttgtctcagtggcaggagcttgagttgcaggctttgatattcaggtacatgttggctggtgctgctgttcctcctgaactccttcaaccaatcaagaaaagccttcttcattctccacactattacctccatcaccctctccaacattaccaaccttctgctttgttgcaatcagggtattggggtagaggagcgatggatccggagccagggcggtgccggagaaccgacggcaagaagtggcgctgttcgagggacgtggtggctgggcaaaagtactgtgagcgccacatgcaccgtggaagaaaccgttcaagaaagcctgtggaactacccacaccaactagtgctattaacaattgtggtgtaactggagttggatccctaggaccaggtgcttcatcatcttccatttgttcaccacccttagcttctgcttcattcaaatctccttttgatcttcatcttgatgaacgttcctctgggaccaagaatgaagacgaagatcatgtgggtggggatggcagatcaggtggaggtggtggccatatgctgaggcatttcttcgatgattggccacgatcactccaagactctgacaacgttgaaaacaatgctgctgctggccgtagcctctctatttcaatgcccggtgcttcctcggatgtgtcattgaaattgtccacgggctatggagaggactcgggcccaggaaatgagaatgtaagcctcgagccagagcagctgcagttgaattgggccggaggatgggcctcgtctaatcaagtggcttcgatgggaggtccacttgctgaggcactcagatcatctacttcaacctcatctcccactagtgttttgcatcgtcacttgcctcgtggatctgagaccagctttattagcacctga

SEQ ID NO: 114 - DNA, 1407 bp, Glycine max
  source 1..1407: mol_type = other DNA
atggaagctaagcctcttcgaactgttccctcttcacacaacacttctggggtaggaggtggaccccagaagaagattggcatggaacacaagatggaaagtcgtgttgttgatgagaggaagagggttgttatggtcaaggaagagaacagcacttcttctcccaacagtgttgaacttcatctcgggtttgatgcttctcattcgactccacaagaaataaaccatgtgatcactgaagctcagaggcgtgagcttcaccatcaagttttcatcttcaaccatttggcttataatcttcctcctccttatcaccttgtgcaattccctagcaacatgtcagaatacagtttcctgggttttgatcatgggattatggtggatccagaacctcataggtgtagaagaactgatggaaagaaatggaggtgtggtaagaatgtggtgcctaaccagaagtactgtgaaaggcacatgcacagaggtcgaaatcgttcaagaaagcctgtggaaacatctcaagttaactctcctttggcaacaaagccttgtagcaagtcacacaccaaaccatcctcaaagacacaatttgaaatttcaagtccaaaccttatcgccattccacatattgacacatcaagtaccccatcaaggagcctcagtgtcaccaattgctcttctgctaataataggtcgaaaaatagggcaggttatgctgactacctcatgtcgttttcttccgcgtccacagtgtcccctggaaccactcttgccactccagttgcccctaagatagctgccttcagcagtgcgacatccgttgttgcttcagatagcaaaagttgcctaaagatatgccagaaagataaccagtccaagagctgtatcagcaacaacatcggtgttaaaagcggtgggaaaggaagcattgttggtgatactaatactaatggcatctctactggaataagcttctccccaacgagtgttcttcaagtttcagtttctggttgcaacccttcatacctgaatgacggaaccaacatagaagctgcatccggtaggtgccggagaacagatggtaagaagtggcagtgcaagagtgctgttcttcctggtcagaaatactgtgccacacacatgcatagaggtgccaaaaagcgtctcacaagccatgaaccagcagctactgctaccagttccgccgttaccgttgcgcggttgccttactcttcagccactaccaacatacagaaagcgcattgtgcaattccaaacacaaatctttccatgtcagtccaagccagggaagcattcatacaatgtaatgagaaaagtcaaagtagcagtgacactgatactactatcagtgacaccttgaatgagtatagctatgcttctttctga

SEQ ID NO: 115 - DNA, 1083 bp, Glycine max
  source 1..1083: mol_type = other DNA
atgatgagtgcaagtgcaggtgcaagaaataggtctccgttcacacaaattcagtggcaagagcttgagcaacaagctcttgtttttaagtacatggttacaggaacacctatcccaccagatctcatctactctattaaaagaagtctagacacttcaatttcttcaaggctcttcccacatcatccaattgggtggggatgttttgaaatgggatttggcagaaaagtagacccagagccagggaggtgcagaagaacagatggcaagaaatggagatgttcaaaggaggcatatccagactcaaagtactgtgaaagacacatgcacagaggcagaaaccgttcaagaaagcctgtggaagtttcttcagcaacaagcaccgccacaaacacctcccaaacaatcccatcatcttataccagaaacctttccttgaccaataacagtaaccccaacataacaccaccaccaccaccctcttctttccctttctctcatttgccctcttctatgcctattgatcagtcccaacccttttcccaatcctaccaaaactcttctctcaatcccttcttctactcccaatcaacctcctctagacccccagatgctgattttccaccccaagatgccaccacccaccacctattcatggactctgctggctcttattctcatgatgaaaagaattatagcaggcatgttcatggaataagggaagatgtggatgagagagctttcttcccagaagcatcaggatcagctaggagctatacagactcgtaccaacaactatcaatgagctcctacaagtcctattcaaactccaactttcagaacattaataatgatgccaccaccaacccaagacagcaagagcagcaactacaacaacaacaacactgttttgttttagggacagacttcaaatcaacaaggccaagcaaagagaaagaagctgagacaacaacaggtcagagaccccttcaccgtttctttggggagtggccaccaaagaacacaacaacagattcctggctagatcttgcttccaactccagaatccaaaccgatgaatga

SEQ ID NO: 116 - DNA, 1098 bp, Glycine max
  source 1..1098: mol_type = other DNA
atgatgaatggaaggaacaggttcccctttaccccatcacagtggcaagagcttgaacaccaagctctcatctacaagtacatggcttcaggcatttccattccacctgatcttctcttcaccataaaaaggagctattttgattcccctctgtcctcaaggcttttgcctaaccagccacagcactttggatggaactaccttcagatgggtttgggaagaaaaatagaccctgagccaggtaggtgtagaagaactgatggcaagaaatggagatgctccaaagaagcatacccagattcaaagtactgtgagaggcacatgcacagagggaagaatcgttcaagaaagcctgtggaagttttaaaatcaacaacaacaccatcatcatcaacaacaaactcaaatgcttcttctacacaacaagcaatctcatcaatcaccaaaattaatagcactctctcacctcttgcatcatctgagactcaccaacaccaccactatcctcaacactatggctcctttctctatcatcatcaccctccttcaaggtcctctggcattggcttgtcttttgaagacaacagtgctcccttgtttcttgacactggctcatgctctcagtccaacacagactgcaggagtaggtatgtttatggagagaaagaggaggtggatgagcatgctttcttcacagaaccttgtggtgttatgaaaagcttctctgcttcctctatggatgactcatggcaactcacaccattgactatgagctcctcatcttcatcttccaagcagaggagttcctttggcttgtccagtgattactcttgcttgcaacttcagagccactcaaagcagcagcagcaagagcatcatcaagatcagggttgctacatgtttggtgctggtcaagttgtgaaagaagaacctcagaaaacggttcatcgcttctttgatgaatggccacacaaaggaagagaaggctcttggcttgatttggatgacaaatcttccacaacccaactttcaatttccatccccacatcttctcatgatttttcaactttcagttccagaacccaccatgatggttga

SEQ ID NO: 117 - DNA, 1038 bp, Glycine max
  source 1..1038: mol_type = other DNA
atgatgagtgcaagtgcaagaaataggtctcctttcacgcaaactcagtggcaagagcttgagcatcaagctcttgtttttaagtacatggttacaggaacacccatcccaccagatctcatctactctattaaaagaagtctagacacttcaatttcttcaaggctcttcccacatcatccaattgggtggggatgttttgaaatgggatttggcagaaaagtagacccagagccagggaggtgcagaagaacagatggcaagaaatggagatgctcaaaggaggcatatccagactccaagtactgtgaaagacacatgcacagaggcagaaaccgttcaagaaagcctgtggaagtttcttcagcaataagcaccgccacaaacacctcccaaacaatcccatcttcttatacccgaaacctttccttgaccaaccccaacatgacaccaccctcttccttccctttctctcctttgccctcttctatgcctattgagtcccaacccttttcccaatcctaccaaaactcttctctcaatcccttcttctactcccaatcaacctcctctagacccccagatgctgattttccaccccaagatgccaccacccaccagctattcatggactctgggtcttattcgcatgatgaaaagaattataggcatgttcatggaataagagaagatgtggatgagagagctttcttcccagaagcatcaggatcagctaggagctacactgaatcataccagcaactatcaatgagctcctacaagtcctattcaaactccaactttcagaacatcaatgatgccaccaccaacccaagacagcaagagcagcaacaacaacaacactgctttgttttggggacagacttcaaatcaacaagaccaactaaagagaaagaagctgagacagctacgggtcagagaccccttcaccgtttctttggggagtggccaccaaagaacacaacagattcatggctagatcttgcttccaactccagaatccaaaccgatgaatga

SEQ ID NO: 118 - DNA, 1167 bp, Glycine max
  source 1..1167: mol_type = other DNA
atggacttccatctgaaggaatggagaaaccagcatgagtcagaggaacaacaacattctacaaagatgccaaaacttctccctgaatcccatcatcaacagccatctgccactgcactccctttgtttgtacctgaacccaacagcagcagcaaagtcagcaccctgtcagattcaacattagcagctgaaactgaaacaatgaccactacaaccactaacagattatttcccaggatggggagctacttcagcttgtctcagtggcaggagcttgagttgcaggctttgatattcaggtacatgttggctggtgctgctgttcctcctgaactccttcaaccaatcaagaaaagccttcttcattcccctcactatttcctccatcaccctctccaacattaccaacctgctgctttgttgcaatcagggtattggggtagaggagcgatggatccggagccagggcggtgccggagaaccgacggcaagaaatggcggtgctcgagggacgtggtggctgggcaaaagtactgtgagcgccacatgcatcgtggaagaaaccgttcaagaaagcctgtggaactacccacaccaactagtgctaataattgtgatggtggatctctaggactaggtgcttcttcatcttccatttcttcaccacccctagcttctgcttcactcaaatccccatttgatcttcttcgtcttaatgaacgttcctctgggaccaagaatgaagacgaagaccatgtgggtggggatggcagatcaggtggagggggtggccatatgctgaggcatttcttcgatgattggccacgatcactgcaagactctgacaacgttgaaaacaatgctgctggccctagcctctctatttcaatgcccggaaatgctgctgctgcttcctcggatgtgtcattgaaattgtccacgggctatggagaggacccaggcccaagaaatgagaatgtgggcctcgtggcagagcagctgcagttgaattgggccggaggatgggcctcgtctaatcaagtggcttccatgggaggaccactggccgaggcactcagatcatctatttcaacttcatctcccactagtgttttgcatcacttgcctcgtggttctggatctgagaccagcattattagcacctga

SEQ ID NO: 119 - DNA, 1782 bp, Glycine max
  source 1..1782: mol_type = other DNA
atgggtgaattatttggcgttgggaaaagaagggacatcatcagcaacaacaacagtgctagtagtagcgttcttgggttggatgtgaaggtgcagagccctgaagtattattccataataggatgaccatgatggggcatcataatcatcacctccatccattgtcatccccctttgataataataacgatatttcctgtgctgctggtgatggcgatggtgatggtcccacgacttacatgtctctcactaatcatataaaccttgttagtggtgcttctgttcttggtcctgctattgatggtggctgtggtgctgctcctgctgcacctgtaagaactttgcagccttttcaaatttcttcttatacttctcccaccaccactaccacagccttcgaacccttttcaggtgtgatggcggcttcattggggtttcccttcacaagtgcacaatggagggagcttgaaaggcaggctatgatatacaagtacatgatggcttcagttcctgttccacctgatctcctcattcctacttcactcacatcctcttctcgctcttcctgcatggatggtggtttcaatctgaggttggcaaatagcactgaccctgagccaggtaggtgcagaagaacagatggaaaaaaatggagatgttcaagagacgtggctcctaaccacaagtactgcgagcgccatatgcatagaggccgtccccgttcaagaaagcctgtggaagttaacaccaacaccactcccactagcatcaacaacaacaataacaaccaccaaatcaaaagggctcgccatgagtgtaataatccctttgctacacctgatgttgctgtggctatttccaatcccacttccagaaaaggtgaatcttcatcccattttcttgggtctactaccactcagccataccttaaatctcccctctgtcttgctaacttcggtctcaaagctgcgagttttgactccgtggcttctgtttctgcaaataaggaacccaggggtttggagtggatgctgaatggagatcctatttccctggatgcttctaactcccaatggcagtctctgatgcataataaagttggaatgagcagtgttagttcctgtaataccaccgagtctcagtatctgaattcatttgcactatatagctctggactggaacaacagaacagacgccatcctttgttcctgaatcctcttcttgttcccatgaaaaatctccaaccagagaaaccaaggggtttcattgatgcttggtctaatgcagaaacagctcaaagcaatgccaacaccaacaacaagaactctgctgcatcaattggtaaattatccctctcttctcttgatctatcaatggggggtggtgctgtgagtgaagatgttgataatgttaacatgggtttgggactaatggagacttatggaaaaacacacactgatactaaaatctctctctcaaattggcacaacccggcaccttgggtggcttcaagttcaacacttgggggtcctctagctgaagttctaaggtcaagcacagtcactgccacgaccaatgaagcaacctccaacgcaacctcatcaccagccaccactacacatgctgaatctaacagccctctgggaacaatggtgtcatctccatccggggtgttgcagaaaacccttatttcattgtctgatagcagtaacaatagcagccctacggttgcatcatcaagggccaattctgagatggccttcctcagctttaaattctag

SEQ ID NO: 120 - DNA, 1011 bp, Glycine max
  source 1..1011: mol_type = other DNA
atggacttgcagttgaagcaatggagaaaccagcatgagtcagagcaagaacaagaacattattccccaaacatggcaaaatttctatctcaacaacaacacccaccaccatttccctctgcactccctctctttgtacctgaacaacccaacaccaaagtcagcaccttgtcagcattttctgattccacattaccctcttctcccagatttcccagaatggagagttgcttcagctttgcacaatggcaagagcttgagttgcaggctctgatattcaggtacatgctggccggtgctcctgttcctcctgagctccttctaccaatcaagaaaagcttccttcaactttatcaccctcctaatttgttggaatcagggtactactggcgaagagaagcactggatccggagccggggcggtgccggaggaccgacggcaagaagtggcggtgctcgaaggacacggtggcaggtcagaagtactgcgaccgccacatgcaccgtggccggaaccgttcaagaaagcctgtggaacaacgtgaaggatctctttctgctatagactctgtttcttcttcacactctgcttcattcaatctccttcacctcggtcaaagttccgctgtggccaagagtgacagcaagagcttgtctagaaaccgtgatcatgtggatggggatggcaaatcaaatggccatgtcttgaggcatttctttgatgattggccaaggacactgcaagagcctgacaatggtgaaagcaatggaagccagaacaacaactcaggaaaatgtctttctatgtcaacaccaggaatcgatccctcggatgtgtcgttgaaattgaccactggctatggagaggacgcgtgccaggcagcttcggtgggaggaccacttgcagaggcattgagatcatccaccaccagctccacttcttcaccaaccagtgttctgcttcagttgcctcctagttctgcttgtgagaccagcttcattagcacctaa

SEQ ID NO: 121 - DNA, 62 bp, synthetic construct
  misc_feature 1..62: note = Synthetic GRF
  source 1..62: mol_type = other DNA
cacatgcaccgtggccgcaaccgttcaagaaagcctgtggaatcacaaactatgactcagtc

SEQ ID NO: 122 - DNA, 62 bp, synthetic construct
  misc_feature 1..62: note = Synthetic GRF
  source 1..62: mol_type = other DNA
catatgatacggcggcgttaccgttcaagaaagcctgtggaatcatctcaaactcactcttc

SEQ ID NO: 123 - DNA, 62 bp, synthetic construct
  misc_feature 1..62: note = Synthetic GRF
  source 1..62: mol_type = other DNA
cacatgcacagagggaaaaatcgttcaagaaagcctgtggaagttttgaaaacaacaccaac

SEQ ID NO: 124 - DNA, 62 bp, synthetic construct
  misc_feature 1..62: note = Synthetic GRF
  source 1..62: mol_type = other DNA
cacatgcatagaggtcgaaatcgttcaagaaagcctgtggaaacatctcaaattaactctca

SEQ ID NO: 125 - DNA, 62 bp, synthetic construct
  misc_feature 1..62: note = Synthetic GRF
  source 1..62: mol_type = other DNA
cacatgcacagagggaagaatcgttcaagaaagcctgtggaagttttgaaaacaacaacacc

SEQ ID NO: 126 - DNA, 62 bp, synthetic construct
  misc_feature 1..62: note = Synthetic GRF
  source 1..62: mol_type = other DNA
catatgatacgacgtcgttaccgttcaagaaagcctgtggaatcatctcaaactcactcttc

SEQ ID NO: 127 - DNA, 62 bp, synthetic construct
  misc_feature 1..62: note = Synthetic GRF
  source 1..62: mol_type = other DNA
cacatgaaccgaggacgccatcgttcaagaaagcctgtggaaggccaatcaggccatgccct

SEQ ID NO: 128 - DNA, 62 bp, synthetic construct
  misc_feature 1..62: note = Synthetic GRF
  source 1..62: mol_type = other DNA
cacatgcacagagggaaaaaccgttcaagaaagcctgtggaagttttgaaaacaacaccaat

SEQ ID NO: 129 - DNA, 62 bp, synthetic construct; identical to SEQ ID NO: 127
  misc_feature 1..62: note = Synthetic GRF
  source 1..62: mol_type = other DNA

SEQ ID NO: 130 - DNA, 62 bp, synthetic construct
  misc_feature 1..62: note = Synthetic GRF
  source 1..62: mol_type = other DNA
cacatgaatagaggacgccatcgttcaagaaagcctgtggaaggccaattaggccatgccct

SEQ ID NO: 131 - DNA, 62 bp, synthetic construct
  misc_feature 1..62: note = Synthetic GRF
  source 1..62: mol_type = other DNA
cacatgaacagaggacgccatcgttcaagaaagcctgtggaaggccaatcaggccatgccct

SEQ ID NO: 132 - DNA, 62 bp, synthetic construct
  misc_feature 1..62: note = Synthetic GRF
  source 1..62: mol_type = other DNA
cacatgcaccgtggccgcaaccgttcaagaaagcctgtggaatcacaaactatgactcactc

SEQ ID NO: 133 - DNA, 62 bp, synthetic construct
  misc_feature 1..62: note = Synthetic GRF
  source 1..62: mol_type = other DNA
catatgcatagaggccgtccccgttcaagaaagcctgtggaagttaacaccaacagcaccac

SEQ ID NO: 134 - DNA, 62 bp, synthetic construct
  misc_feature 1..62: note = Synthetic GRF
  source 1..62: mol_type = other DNA
cacatgcaccgtggccgcaaccgttcaagaaagcctgtggaatcacaaactatgacacagtc

SEQ ID NO: 135 - DNA, 62 bp, synthetic construct
  misc_feature 1..62: note = Synthetic GRF
  source 1..62: mol_type = other DNA
cacatgcatcgtggccgaaaccgttcaagaaagcctgtggaacaacgtgatggatctctttc

SEQ ID NO: 136 - DNA, 62 bp, synthetic construct; identical to SEQ ID NO: 132
  misc_feature 1..62: note = Synthetic GRF
  source 1..62: mol_type = other DNA

SEQ ID NO: 137 - DNA, 62 bp, synthetic construct
  misc_feature 1..62: note = Synthetic GRF
  source 1..62: mol_type = other DNA
cacatgcaccgtggaagaaaccgttcaagaaagcctgtggaactacccacaccaactagtgc

SEQ ID NO: 138 - DNA, 62 bp, synthetic construct
  misc_feature 1..62: note = Synthetic GRF
  source 1..62: mol_type = other DNA
cacatgcacagaggtcgaaatcgttcaagaaagcctgtggaaacatctcaagttaactctcc

SEQ ID NO: 139 - DNA, 62 bp, synthetic construct
  misc_feature 1..62: note = Synthetic GRF
  source 1..62: mol_type = other DNA
cacatgcacagaggcagaaaccgttcaagaaagcctgtggaagtttcttcagcaacaagcac

SEQ ID NO: 140 - DNA, 62 bp, synthetic construct
  misc_feature 1..62: note = Synthetic GRF
  source 1..62: mol_type = other DNA
cacatgcacagagggaagaatcgttcaagaaagcctgtggaagttttaaaatcaacaacaac

SEQ ID NO: 141 - DNA, 62 bp, synthetic construct
  misc_feature 1..62: note = Synthetic GRF
  source 1..62: mol_type = other DNA
cacatgcacagaggcagaaaccgttcaagaaagcctgtggaagtttcttcagcaataagcac

SEQ ID NO: 142 - DNA, 62 bp, synthetic construct
  misc_feature 1..62: note = Synthetic GRF
  source 1..62: mol_type = other DNA
cacatgcatcgtggaagaaaccgttcaagaaagcctgtggaactacccacaccaactagtgc

SEQ ID NO: 143 - DNA, 62 bp, synthetic construct
  misc_feature 1..62: note = Synthetic GRF
  source 1..62: mol_type = other DNA
catatgcatagaggccgtccccgttcaagaaagcctgtggaagttaacaccaacaccactcc

SEQ ID NO: 144 - DNA, 62 bp, synthetic construct
  misc_feature 1..62: note = Synthetic GRF
  source 1..62: mol_type = other DNA
cacatgcaccgtggccggaaccgttcaagaaagcctgtggaacaacgtgaaggatctctttc

SEQ ID NO: 145 - DNA, 22 bp, Glycine max
  source 1..22: mol_type = genomic DNA
ccgttcaagaaagcctgtggaa

SEQ ID NO: 146 - DNA, 22 bp, Glycine max
  source 1..22: mol_type = genomic DNA
tcgttcaagaaagcctgtggaa

SEQ ID NO: 147 - DNA, 1737 bp, Triticum aestivum
  source 1..1737: mol_type = other DNA
atggacctgggcggggtgctgatggcggccgcggacgcgggggtgggcggcggggacctcggcatgctcggatctaggctgctcaagcacgggaggggcaatgaggccgacgagcacggctggggcggcggcaggccggcgtccaagcaggcccgggtcgccggggacagcgacgcggtgtccgaggccgtcaaggcggccgcgccctacctgctcggcacctgcagccccgggcacggccgggagaagatgctcagcttctcctcctcgcagccggcctcctgcccctccgccgctcaggccgcgctgccgctctactacggcacgcccgcttcttgctcagggttgagctcagtgagggggcccttcacgccgtcgcagtggatggagctggagcaccaggccctgatctacaagtacctggcggccaacatcgccgtgcctcacaacctcgtcgtccccatccgccggagcgtcacctcgctctacccgtccgcctactttggctcctccacattggggtgggggcctttccagctgggctactccgggagcgcggacctggagcccgggcggtgccgccggacggacggcaagaagtggcggtgctccagggacgccgtcgccgaccagaagtactgcgagcggcatatgaaccggggacgccatcgttcaagaaagcatgtggaaggccagcctggccatgccgcgaaagcgatgcctgcgacggccgctgctgccgcccagcccggtgctctcgccaccgggggcggcggcggagctcccgccggcgccgccatctgccacgagcagcaaccgttgaagaactacgccgccagcaccattgatccttgttcactgcaatataacagggaaatggtgagcaagcagcaacacgagtgcgagcaagtgcaggactccgacaccctctcgatgctgacctccatgagcgcgaggaacaccaatgcaggcagcatgttcccgttctcaaaggaacatcataaccacaatcctttcgaggtgacgagctcgaggccggactacgggctggtttcatccgactcgctgatgagctcccctcacagctccctggagaacgtcaacctgctcacctcgcagcgagctctctcgagcgagcagcagagctcgctctccctgcagcacttcgcggactggccgaggacgccctcgcagcaggggcagggaggaggtctctcatggccggacgccgaggacatgcaagctcatcagaggacccagctctcggtgtccgccgttccaatggcgtcctctgacctgtcgtcggcctccacgtccccgatccacgagaagctcatgctgtcgcccctcaagctgagccgcgagtacagccccatcggcctcagcgtcgcggccacggcggcggtggcgaaggacgagggggaggcgaactggatgcccatgttccgcgactcgtccatgggcgggccgctgggggaggctctgaacaagaacaatggcggcaacatggaggccaagagctacctgtcggcgtcgctgaacctgatgacggacgcctgggactcgagcccgctggagtcgtcgccggtgggggtcctgcagaggaccgccttcggatcggtgtccagcagcaccggcagcagccccaggcaggagtaccacggcgtgtatgatggtaacccgcgggatgatctcggctccatcgtcgtgaatcaccccagcatccgcctgatgtga

SEQ ID NO: 148 - DNA, 678 bp, Triticum aestivum
  source 1..678: mol_type = other DNA
atgaccgagcgaaggcaggagcactcgccgccgtccaagatcccccgcctctccggcgccgacgacgatgacggcgcagggacggtgaccatggcggcgccgtcgccgctggttcttgggctgggtctcggcgtaggcggcagcagcagtgacagcggacgcggcgacgcggaggcctctgcggcgacgcggccgtcggcgctgacgttcatgcagcggcaggagctggagcaccaggtgctcatctaccgctacttcgccgccaacgctcccgtgcccgtgcacctcgtgctccccatctggaagagcgtcgccgcttcctcctccgccccgcagaggtttccatccctggcggggctggggagcatgtgctacgaccacaggagcagcatggagccggagccggaccggtgccggcgcacggacggcaagaagtggcggtgctcgcgcggcgtggtgccggggcacaagtactgcgagcgccacgtccaccgcggccgcagccgtgcaagaaagcctgtggaagccgcggcggccacatcagccgtcccgatccgcgcgatgcacgccgccgacgcgcagggcgccaccagtgcgcacgcggcgccaccgcagcgcctcggcttctcctcccccgccggcgtctacctggcgcacggcaccgcccgtgccacctga

SEQ ID NO: 149 - DNA, 1176 bp, Triticum aestivum
  source 1..1176: mol_type = other DNA
atggcgatgccctttgcctccctgtcgccggcagccgaccaccaccgctcctcccccatcttccccttctgccgctcctcccctctctactcggcaggggaggaggcggcgcagcagcagcagcagcacgcgatgagcggcgcgaggtgggcggcgatgaggccggcgaccttcacggcggcgcagtaccaggagctggagcagcaggcgctcatctacaagtacctcgtcgccggcgtgcccgtcccgccggatctcctcctccccatccgccgcgacttcgactccctcgcctcgcgcttctaccaccaccacgcccttgggtacgggtcctacttcgggaagaagctggatccggagccggggcggtgccggcggacggacggcaagaagtggcggtgctccaaggaggccgcccaggactccaagtactgcgagcgccacatgcaccgcggccgcaaccgttcaagaaagcctgtggaaacgcagctcgtcgccacgccccactcccactcccactcccagcagctgcagcagcacgcccccgccgccaccgccgccgcgttccacagccactcgccgtacccggcgatcgccactggcggcggcggcggcgcggccggctccttcggcctggggtctgctcagctgcacatggacaatgctgctgcgccttacgcgaccgctggtgcggccggaaacaaggatttcaggtattctgcctatgggtttaggacttcggcgctggaggagcacaaccagttcatcagcgcggccatggacaccgccatggacaactactcatggcgcctgatgccggcccagaactcggcgttctcactctcgagctaccccatgctgggcaccctgggcgacctggaccagagcgcgatctgctcgctggccaagacggagagggagccgctgtccttcggcggcggcggcggcttcgaggacgacgagtcggcggtgaagcaggagaaccagacgctgcggcccttcttcgacgagtggcccaaggacagggactcgtggccggagctgcaggaccatgactccaaccacaacagcaacgccttctcggccaccaagctgtccatctccatcccggtgaccagctccgacttctccaccaccgccggctcccgctcgccccacggtatatactcccggtga

SEQ ID NO: 150 - DNA, 1824 bp, Triticum aestivum
  source 1..1824: mol_type = other DNA
atggacctgggcggggtgctgatggcggccgcggacgcgggggtgggcggcggggagctcggcatgctcggatctaggctgctcaagcacgggaggggcaatgccgcggcggcggaggccgacgagcgtcacgagcacgggtggggcggcggcaggccggccgccaagcaggcccgggtcgccggggacagcgacgccgtgtccgaggccgtcaaggcggccgcgccatacctgctgggcacctgcagccccgggcacggccgggagaagatgctcagcttctcctcctcgcagcagccgccctcctgcccctccgccgccgccgccgctcaggccgcgctgccgctctactacggcacgcccgcttcttgcttagggttgagctcggtgagcttgagcgccagcatccagggcgccatggccagggtgagggggcccttcacgccgtcgcagtggatggagctggagcaccaggccctgatctacaagtacctggcggccaacatcgccgtgccgcacagcctcctcgtccccatccgccggagcgtcacctcgctctacccgtccgcctactttggctcctccacattggggtgggggcctttccagctgggctactccgggagcgcggacctggagcccgggcggtgccgccggacggacggcaagaagtggcggtgctccagggacgccgtcgctgaccagaagtactgcgagcggcatatgaaccggggacgccatcgttcaagaaagcatgtggaaggccagcctggccatgccgcgaaagcgatgcctgcgacggtggcggctgctgccgcccagcccggtgctctcgccaccgggggcggcggcggagccaccgccggcgccgccatctgccacgagcagcagccgttgaagagctactccgccagcaccattgatccttgttcactgcaatacaacagggaaatggcgagcaagcagcaacacgagtgcgagcaagtgcaggactcggacaccctctcgatgctgacctccatgagcgcgaggaacaccaacacgggcagcatgttcccgttctcaaaggaacatcataaccacaatcctttcgaggtgacgagctcgaggccggactacgggctggtttcctccgactcgctgatgagctccccccacagctccctggagaacgtcaacctgctcacctcgcagcgagcgctctcgagcgagcagcagagctcgctctccctgcagcacttcgcggactggccgaggacgccgtcgcagcaggggcagggagggggaggcctctcatggccggacgccgagaacatgcagctggctcatcagcggacccagcagctctcggtgtccgccgctccgatggcgtcctccgacctgtcgtcggcctccacgtcccccatccacgagaagctcatgctgtcgcccctcaagctgagccgcgagtacagccccatcggcctcagcgtcgcggccacggcggcagcggcggcgaaggacgagggggaggcgaactggatgcccatgttccgcgactcgtccatgggcgggccactgggggaggctctgaacaagaacaatggcggcaacatggaggccaagaactacctgtcggcgtcgctgaacctcatgacggacgcctgggactcgagcccgctggagtcgtcgccggtgggggtcctgcagaggaccgccttcgggtcggtgtccagcagcaccggcagcagccccaggcaggagtaccacggcgtgtatgatggtaatccgcgggatgatctcggctccatcgtcgtgaatcaccccagcatccgcctcatgtga

SEQ ID NO: 151 - DNA, 690 bp, Triticum aestivum
  source 1..690: mol_type = other DNA
atgaccgagcgaaggcaggagcactcgccgccgtccaagctcccccgcctctccggccccgacgccgacgccgacgacaatgatggcgcagggacggtgaccatggcggcgccgtcgccgctggttcttgggctgggtctcggcgtaggcggcagctgcagtgacagcggacgcggcgacgcggaggcctctgcggcgacgcggccgtcggcgctgacgttcatgcagcggcaggagctggagcaccaggtgctcatctaccgctacttcgccgccaacgctcccgtgccggtgcacctcgtcctccccatctggaagagcgtcgccgcctcctcctccgccccgcagaggttcccatccctggcgggactggggagcatgtgctacgaccacaggagcagcatggagccggagccggaccggtgccggcgcacggacggcaagaagtggcggtgctcgcgcggggtggtgccggggcacaagtactgcgagcgccacgtccaccgcggccgcggccgtgcaagaaagcctgtggaagccgcggcggccacatcagccatcccgatccgcgcgatgcacgccgccgacgcgcagggcgccaccagcgcgcacgcggcgccgccgcagcgcctcggcttctcctcccccgccggcgtgtacctggcccacggcaccgcccgtgccacctga

SEQ ID NO: 152 - DNA, 1248 bp, Triticum aestivum
  misc_feature 46..51: note = n is a, c, g, or t
  source 1..1248: mol_type = other DNA
atgcttcctgagctcaccgccgccgccatggagctcgggcaggtgnnnnnntacacgccgccggcgaccaaggacgcgagatccggcggcggcttcgcccaggccgccgcttgcccctacccctacccctcccccttcctcgacgagcagaagatgctcagcttctccaaggccgccgcccctccatcgtcaggtatggattttggcaggtccaatgagcagaggctgctgctggccaggagcaagatgcccttcactccttcacagtggatggagctggagcaccaggccctcatatacaagtatctcaatgcaaaggcccccataccttccagcctgctcatctccatcagcaaaagcttcagaccctcctccgatagaatgccctggaggcctgtctaccaagggttcaccaatgcagattctgacccggaacctggaagatgccgtcgaacagacggcaagaaatggcggtgctcaaaggaggcgatggccgagcacaagtactgtgagcggcacatcaataggaaccgccatcgttcaagaaagcctgtggaaaaccaaacaaggaagaacgccaaagagacgcctgctgctggctcgttatcggccgctgtctcacagggtggctgtaagaaagcaaaagctggtgatgaactgaagccagggagcgtcagctattggacagataatttaaacagggcaatggtgagcaaagccaggggaaacaaccctgaagaaggcaacagtgctccactcctgaattctactaatcaacaacacacattgtccttgttctctcaactgaagcaacagagcaaaccagataagttcagcccggcagtcgatagtgaatcgatctcctcaaatacagtattgaagccctgggaaagaagcaaccagcagagcagcaaggacgtttcttcgacgacgctccatgatcgcgggtgccttcaatcagtccttcaagatttcagcatgcataagaatgacaagatcgaggctcagaaaaacaatgcttcagtgccatcaactttctattcacctacagaaggtcaacacatcagctgccttgcatctaacatgatgcaagtgcaggaggattgcatctcaagctcttgggagatacctcaaggtgggccattaggtgaaatcctaacaaactccaagaacactgatgacttgaccaataagtgtgaatcaagatcatatggttggttactgagtcttgatgaacatgaaatgtga

SEQ ID NO: 153 - DNA, 1164 bp, Triticum aestivum
  source 1..1164: mol_type = other DNA
atggcgatgccctttgcctccctgtcgccggcagccgaccaccaccgctcctcccccatcttccccttctgccgctcctcccctctctactcggtaggggaggagacggcgcatcagcagcagcagcagcacacgatgagcggcgcgaggtgggcggcgaggccggcgaccttcacggcggcgcagtacgaggagctggagcagcaggcgctcatctacaagtacctcgtcgccggcgtccccgtcccgccggatctcctcctccccatccgccgcggcttcgactccctcgcctcgcgcttctaccaccaccacgccctcgggtacggttcctacttcgggaagaagctggatccggagccggggcggtgccggcggacggacggcaagaagtggcggtgctccaaggaggccgcccaggactccaagtactgcgagcgccacatgcaccgcggccgcaaccgttcaagaaagcctgtggaaacgcagctcgtcgccacgccccaccactcccactcccagcagctgcagcagcacgcccccgccgccagcgccgccgcgttccacagccactcgccgtatccggcgatcgcctctggcggcggcggctccttcgccgtgggatctgctcagctgcacatggacaatgctgcttcgccttacgcgaccgctggtgccgccggaaacaaagatttcaggtattctgcctatgggtttaggacttcggcgatggaggagcacaaccagttcatctctgcggccatggagaccgccatggagaactactcatgccgcctgatgccggcccagaactcatccttctcactcgccagctaccccatgctgggcaccctgggcgaccttgaccagagcgcgatctgctcgctggccaagacggagagggagcctctgtccttcttcggcggcggcggcggcttcgacgacgacgactcggcggtgaagcaggagaaccagacgctgcggcccttcttcgacgagtggcccaaggacagggactcgtggccggagctgcaggaccacgatgccaacaacagcagcaacgccttctcggccaccaagctgtccatctccatcccggtgaccagctccgacttctccaccaccgccggctcccgctcgcccaacggtatatactcccggtga

SEQ ID NO: 154 - DNA, 1107 bp, Triticum aestivum
  source 1..1107: mol_type = other DNA
atgctgagctcgtcggcggcgatggggatggggctgggcgggtacggccagcagcagcagcagcagatgcagatgcagatgcagcggggggcggagccggtgttcacgccggcgcagtgggccgagctggagcagcaggcgctgatttacaagtacctcatggcgggcgtgcccgtgccgcccgatctcctgctccccatccgcccccaccccgccggcgccgccggaaccaccttctccttcgccaaccccgccgcctcgcccttctaccaccaccaccacccctccatgagttactacgcctactatggcaagaagctcgacccggagccgtggcggtgccgacgcaccgacggcaagaagtggcggtgctccaaggaggcgcaccccgactccaagtactgcgagcgccacatgcaccgtggccgcaaccgttcaagaaagcctgtggaatccaagtctgcttcccctgcgcaccagtcgcagcagccccagttgtccgccgtcacgtccgcggcccgcgacgccgagcctctcccctccctcccggcgggggctaaaacccatggcctgtccctcggcggggctggctcgtcgcagatgcacgtcgacgcctcgtcatacggcggcaaatactcccttggagctaaatctgatgtgggtgaactgagcttcttctctggagcatcaggaaacaacaacaggggattcaccatcgattccccaacggacagctcgtggcactcaatgggatccagcctgaccccataccaactgtcgaaacctagagattccggcctcatgcagggcggcttctcgtattcccactttgagccgtcgcaggagctcgggcaggtaaccatcgcctcgctgtcccactcccaggagcaggaccgccgctctttcgggggcggtggtggaggtgggggtggaggggcagggctcatgggaaatgttaagcaggagaaccagccgctgaggcccttcttcgacgagtggccggggaggcgggactcgtggtcggagatggacgacgagcgctccaacggcacctccttctcgacgacccagctctcgatctccatcccaatgcctcgatgcgattga

SEQ ID NO: 155 - DNA, 1227 bp, Triticum aestivum
  source 1..1227: mol_type = other DNA
atggagctcgggcaggtgctgggctacacgccaccggcgaccaaggacgcgagatccggcggcggcttcacccaggctgccgcttgcccctacccctacccctcccccttcctcgacgagcagaagatgctcagcttctccaaggccgccgccgctcaccagccgccctcaggtatggattttgggaggtccaatgagcagaggctgttgctggccaggagcaagatgcccttcaccccttcacagtggatggagctggagcaccaggccctcatttacaagtatctcaatgcaaaggcccccataccttccggcctgctcatctccatcagcaagagcttcagaccctcctccgatagaatgccctggaggcctgtctatcaagggttcaccaatgcagattctgacccggaacctggaagatgccgtcgaacagacggcaagaaatggcggtgctcaaaggaggcgatggccgagcacaagtactgtgagcggcacatcaataggaaccgccatcgttcaagaaagcctgtggaaaaccaaacaaggaagaacgccaaagagacacctgctgctggctcgatatcggccgctgtctcacagggtggctgtaagaaagcaaaagctggtgatgaactgaagccagggagcgtcagttattggacagataatttaaacagggcaatggtgagcaaagccaggggaaacaaccctgaagaaggcaacagtgctccactcctgaattctactaatcaacaacacacattgtccttgttctctcaactgaagcaacagagcaaaccagataagttcagcccggcagtcgatagtgaatcgatctcctcaaatactgtattgaagccttgggaaagaagcaaccagcagagcagtaaggacgtttcttccacgacgctccatgatcgcgggtgccttcaatcagtccttcaagatttcagcatgcataagaatgacaagatcgagtctcagaaaaacaatgcttcagtgccatctactttctattcatctacagaaggtcgacacatcagctgccttgcatctaacatgatgcaagtgcaggaggattgcatctcaagctcttgggagatacctcaaggtgggcctttaggtgaaatcctaacaaactccaagaatactgatgacttgaccaataagtgtgaatcaagatcatatggttggttactgagtcttgatgaacatgaaatgtga

SEQ ID NO: 156 - DNA, 1836 bp, Triticum aestivum
  source 1..1836: mol_type = other DNA
atggacctgggcggcgtgctgatggcggccgcggacgcgggggtgggcggcggggacctgggcatgctcggatctaggctgctcaagcacgggaggggcaatgcggcggcggcggaggccgacgagcacggctggggcagcggcaggccgcccgccaagcaggcccgggtcgcggcctcggccgcgtccggggacagcgacgccgtgtccgaggccgtcaaggcggcggcaccctacctgctcggcacctgcagccccgggcacggccgggagaagatgctcagcttctcctcctcgcagccgccctcctgcccctcctccgccgccgccgccgccgctcaggccgcgctgccgctctactacggcacgcccgcttcttgctcagggttgagctcggtgagcttgagcgccagcatccagaacgccatggccagggtgagggggcccttcacgccgtcgcagtggatggagctggagcaccaggccctgatctacaagtacctggcggccaacatcgccgtgcctcacaacctgctcgtccccatccgccggagcgtcacctcgctctacccgtccgcctactttggctcctccacattggggtgggggcctttccagctgggctactccgggagcgcggacctggagcccgggcggtgccgccggacggacggcaagaagtggcggtgctccagggacgccgtcgccgaccagaagtactgcgagcggcatatgaaccggggacgccatcgttcaagaaagcatgtggaaggccagcctggccatgccgcgaaagcgatgcctgcgacggtggcggcggctgctgcccagcccggtgctctcgccaccgggggcggcggcggagctaccgccggcgccgccgccatctgccacgagcagcagccgttgaagaactacgccgcgaacaccattgatccttgttcactgcaatataacagggaaatggtgagcaagcagcagcaacacgagtgcgagcaagtgcaggactccgacaccctctcgatgctgacctccatgagcgcgaggaacaccaacacgggcagcatgttcccgttctcaaaggagcatcacaatcacaatcctttcgaggtgacgagctcaaggccggactacgggctggtttcatccgactcgctgatgagctcccctcacagctccctggagaacgtcaacctgctcacctcgcactcgcagcgagcgctctccaacgagcagcagagctcgctctccctgcagcacttcgcggactggccgaggacgccctcgcagcaggggcaaggaggaggaggtctctcatggccggacgccgaggacatgcaagcacatcagaggacccagctctcggtgtccgccgctccaatggcgtcccccgacctgtcgtcggcctccacgtccccgatccacgagaagctcatgctgtcgcccctcaagctgagccgcgagtacagccccatcggcctcagcatcgcggcgacggcggcggcggcgaaggacgagggggaggcgaactggatgcccatgttccgcgactcgtccatgggcgggccgctgggggaggccctgaacaagaacaatggcggcaacatggaggccaagaactacctgtcggcgtcgctgaacctcatgacggacgcctgggactcgagcccgctggagtcgtccccggtgggggtcctgcagaggaccgccttcgggtcggtgtcgagcagcaccggcagcagccccaggcaggagtaccacggcgtgtatgatggtaacccgcgggatgatctcggctccatcgtcgtgaatcaccccagcatccgcctcatgtga

SEQ ID NO: 157 - DNA, 930 bp, Triticum aestivum
  source 1..930: mol_type = other DNA
atgatgctgggagggcacggcggcggcggcgggaggtgcctgttcacggcgtcgcagtggcgggagctggagcaccaggcgctcatctacaagtacatggccgccggctcgcaggtgccccacgagctggtcctcccgctccgccaccgcgacgccgccgccttcgccggcatcgacaccgccccctccgtcgcctgctaccctcctccgcagccctccctggggtgggggctctacggggcgggggcgcagtacgcgcggaagccggaggacccggagcccgggcggtgccggcggacggacggcaagaagtggcgctgctccagggaggcgtacggggagtccaagtactgcgacaggcacatgcaccgcggcaagaaccgttcaagaaagcctgtggaaccgatgagctcctcctccgtctcctccccggccgcctcctaccgccagaccaccctctccatgtcgccccccacgccggccgacacgcccagctacggccacggccacctccgcgcagctgcttctcagagccagataaaccctctccagctccacctcgacaccccgtcgcccccgccgtcctaccacaggtacgcgccggcgcagcagtacgggggctccttcttcccgagcaggcagcaggtgcaggaggaggccgaggcggaggcgaggcggcggcagcacttcctggctctcggcgccgacctgagcctggacaagccggacgccaccaccgcggcgtcctcgacaaccgaggagaagccgctgcggcgcttcttcgacgagtggccgcgcgacgggaacgccgtcgaggttcggccctggaatatgggccaccgggacgagacgctgctctccatgtccatccccacgacgacggcctcgcaccccgacctcgccgcctaccgccaccacaacgatgaataa

SEQ ID NO: 158 - DNA, 1233 bp, Triticum aestivum
  source 1..1233: mol_type = other DNA
atgatgatgatgggcggtcgcgcgggggccggcggcgtcggggccggcgggggccggtgcccgttcacggcgacgcagtggcaggagctggagcaccaggcgctcatctacaagtacatggcctccggcgtgcccatcccctccgacctcctcctcccgctccgccgcagcttcctcctcgactccgccctcgccacctccccctccctcgccttccctccccaggccgcactgggctggggatgtttcgggatggggttcggccggaaggcggaggacccggagccggggcggtgccggcggactgacggcaagaagtggcgctgctccaaggaggcgtacccggactccaagtactgcgagaagcacatgcaccgcggcaagaaccgttcaagaaagcctgtggaaatgtccttggccacgcccccgccgccgccttcctcctcggcctcctcctcctcctccaacgtccactccgccgtcaacgccgccaccaccaccacctcccccgcgccgtcctaccaccgccacgccgccgcgactcacgacacgacgccctaccacacgctctacggcggcccctactcctccgccggccgccagcagcacgccagcgcctaccaccacgccgcgcaggtcagcccgttccacctgcacctcgacaccacccacccgcacccgccgccgtcctactactccaccatggaccacagcaaggacagctacgcctacgggcacagcgtcaaggaggtgcacggcggcggcgagcacgccttcttctcctccgacgtcaccactgacagggaccaccaccaccaccaacaccacgccggcgccggcggcaacgggcagtggcagttcaagcagctcggcggcatggagcccaagcagcacaaccccacgtcgctcttccccggctgcggcgggtacggcaacaacgcggcgtacgccatcgacctgtccagcaaagaagaggacgaggagaaggagaggcggcagcagcagcagcactgcttcctgctgggcgccgacctgaggctcgacaagccgtcgtcggggcacggcgactccgccgaccagaagcctctccggcccttcttcgacgagtggccgcacgagaagaccgggagcaaggggtcgtggatggggctcgagggggagacgcagctctccatctccatcgccaacgaactccccatcaccaccacctcccgctaccaccatggtgaatga

SEQ ID NO: 159 - DNA, 1230 bp, Triticum aestivum
  source 1..1230: mol_type = other DNA
atggcgatgccgtatgcctctctttccccggcaggcgaccgccgctcctccccggccgccaccgcctccctcctccccttctgccgctcctcccccttctccgccggcggcggcaatggcggcatgggggaggaggcgcggatggacgggaggtggatggcgaggccggtgcccttcacggcggcgcagtacgaggagctggagcaccaggcgctgatatacaagtacctggtggccggcgtgcccgtcccgccggatctcgtgctccccatccgccgcggcatcgaatccctcgccgcccgcttctaccacaaccccctcgccatcgggtacggatcgtacctaggcaagaaggtggatccggagccgggccggtgccggcgcacggacggcaagaagtggcggtgcgccaaggaggccgcctccgattccaagtattgcgagcgccacatgcaccgcggccgcaaccgttcaagaaagcctgtggaaacgcagctcgtcccgcacacccagccgccggccgcctccgccgtgccgcccctcgccaccggcttccacagccactccctctaccccgccatcggcggcagcaccaacggtggtggaggcggggggaacaacggcatgtccatgcccagcacgttctcctccgcgctggggccgcctcagcagcacatgggcagcaatgccgcctctccctacgcggctctcggtggcgccggaacatgcaaagatttcaggtataccgcatatggaataagatctttggcagacgagcacagtcagctcatgacagaagccatgaatacctccgtggagaacccatggcgcctgccgccgtcgtctcaaacgacctcattcccgctttcaagctacgctcctcagcttggagcaacgagtgacctgggtcagaacaacaaccacaacaacagcagcagcaacagtgccgtcaagtccgagcggcagcagccgctctccttcccggggtgcggcgactttggcggcggcggcatggactccgcgaagcaggagaaccagacgctgcggccgttcttcgacgagtggccgaagacgagggactcgtggtcggacctgacggacgacaactccagcctcgcctccttctcggccacccagctgtcgatctcgatacccatgacgtcctccgacttctccgccgccagctcccagtcgcccaacggtatgctgttcgccggcgagatgtactag

SEQ ID NO: 160 - DNA, 1113 bp, Triticum aestivum
  source 1..1113: mol_type = other DNA
atgctgagctcgtcggcggcgatggggatggggctgggcgggtacggccagcagcagcagcagcagcagatgcagatgcagatgcagatgcagcggggggcggggccggtgttcacgccggcgcagtgggccgagctggagcagcaggcgctgatttacaagtacctcatggcgggcgtgcccgtgccgcccgatctcctgctccccatccgcccccaccccgccggcgccggagccaccttctccttcgccaaccccgccgcctcgcccttctaccaccaccaccacccctccatgagttactacgcctactatggcaagaagctcgacccggagccgtggcggtgccgccgcaccgacggcaagaagtggaggtgctccaaggaggcgcaccccgactccaagtactgcgagcgccacatgcaccgtggccgcaaccgttcaagaaagcctgtggaatccaagtctgcttcccctgcgcaccagtcgcagcagcccccgttgtccgccgtcacgtccgccacccgcgacgccgagcctctcccctccctcccggcgggggctaagacccatggcctgtccctcggcggggctggctcgtcgcagatgcacgtcgacgcctcgtcatacggcaacaaatactcccttggagctaaatctgacgtgggtgaactgagcttcttctctggagcatcaggaaacaacaacaggggcttcaccatcgattccccaacggacagctcgtggcactcaatgggatccagcctgcccccgtaccaactgtcgaaacctagagattccggcctcatgcaaggcggcttctcgtattcccactttgagccgtcgcaggagcttgggcaggtaaccatcgcctcgctgtcccactcccaggagcaggaccgccgctccttcggtggcggcggtggtggtggaggtggaggggcagggctcatgggaaatgttaagcaggagaaccagccgctgaggcccttcttcgacgagtggccggggaggcgggactcgtggtcggagatggacgacgagcgctccaacggcacctccttctcgacgacccagctctcgatctccatcccaatgcctcgatgtgattga

SEQ ID NO: 161 - DNA, 684 bp, Triticum aestivum
  source 1..684: mol_type = other DNA
atgaccgagcgaaggcaggaacactcgccgccgtccaagctcccccgcctctccggccccgacgccgacgacaacgacggcgcagggacggtgaccatggcggcgccgtcgccgctggttcttgggctgggtctcggcgtaggcggcagcagcagtgacagtggacgtggcgacgcggaggcatctgcggcgacgcggccatcggcgctgacgttcatgcagcggcaggagctggagcaccaggtgctcatctaccgctacttcgccgccaacgctcccgtgcccgtgcacctcgtcctccccatctggaagagcgtcgccgcctcctcctccgccccgcagaggttcccatccctggcgggactggggagcatgtgctacgaccacaggagcagcatggagccggagccggaccggtgccggcgcacggacggcaaaaagtggcggtgctcgcgcggcgtgatgccggggcacaagtactgcgagcgccacgtccaccgcggccgcggccgtgcaagaaagcctgtggaagccgcgccggccacatcagccatcccgatccgcgcaatgcacgccgccgacgcgcagggcgccacaagcgcgcacgcggcgccaccgcagcgcctcggcttctcctcccccgccggcgtctacctggcccacggcaccgcccgtgccacctga

SEQ ID NO: 162 - DNA, 648 bp, Triticum aestivum
  source 1..648: mol_type = other DNA
atggccgacgagaaagaagccgactcgctgcagccgccgtccaagcagccccgcctctcctccgccgactcgaacgccggtgcggtgacgatggcggtctcgtcgccgctgggtcttggcctcggcctgggactcggcggcgatagccgcggcgagcagcaggccttccaagcacgggcagccgcggcggcggcgaaatcggcgctgacgttcatgcagcagcaggagctggagcaccaggtgctcatctaccgctacttcgcggcgggggcgcccgtgccggtgaacctcgtgctgcccatctggaagagcatcgccgcctcctccttcggcccgcaccgcttcccctccctgattgggcttgggagcctgtgcttcgactaccggagcagcatggagccggagcccgggaggtgccgccgcacggacggcaagaagtggcggtgctcccgcgacgtgctgcaggggcacaagtactgcgagcggcacgtccaccggggccgcggccgttcaagaaagcctgtggaaggagcccccgcagccccggcgcacagcggcagcagcaccaccgccccgccccgcgccatcggcttctcccccgccggcatcctccacgccacccacagcgccgccgcgcgcgccacctga

SEQ ID NO: 163 - DNA, 963 bp, Triticum aestivum
  source 1..963: mol_type = other DNA
atgatgctgggagggcacggcggcggcggcggcgggaggtgcctgttcacggcgtcgcagtggcgggagctggagcaccaggcgctcatctacaagtacatggccgccggctcgcaggtgccccacgagctggtcctcccgctccgccaccgcgacgctgccgccttcgccgccatcgacaccgtcccctccctcgcctgctaccctcctccgcagccatccctggggtgggggctctacggggcgggggcgcagtacgcgcggaagccggaggacccggagccggggcggtgccggcggacggacggcaagaagtggcgctgctccagggaggcgtacggggagtccaagtactgcgacaggcacatgcaccgcggcaagaaccgttcaagaaagcctgtggaaccgatgtcctcctcctccgtctcctccccggccgcctcctaccgccagaccgccctctccatgtcgccccccacgccggccgacacgcccacctacggccacggccacggccacgaccacctccgcgcagctgctggtcagagtcagagccagataaaccctctccagctccacctcgacaccccgtcgcccccgccgtcctaccacaggtacgcgccggcgcagcagtacgggggctccttcttccagaacaggcagcaggtgcaggaggaggcggaggcggaggcgaggcggcggcagcacttcctggctctcggcgccgacctgagcctggacaagcccgacgccaccaccgcggcgtcctcgacaaccgaggagaagccgctgcggcgcttcttcgacgagtggccgcgcgacgggaacgccgtcgagggtcggccctggaatatgggccaccgggacgagacgctgctctccatgtccatccccacgacgacggccacgcaccccgacctcgccgccgcctcgcgctaccaccaccaccacaacgatgaataa

SEQ ID NO: 164 - DNA, 1245 bp, Triticum aestivum
  source 1..1245: mol_type = other DNA
atgatgatgatgggtggtcgcgcgggggccggcggcgtcggggcaggcggggggcggtgcccgttcacggcgacgcagtggcaggagcttgagcaccaggcactcatctacaagtacatggcctccggcgtgcccatcccctccgacctcctcctcccgctccgccgcagcttcctcctcgactccgccctcgccacctccccctccctcgccttccctccccaggccgcacttggctggggttgctttggcatggggttcggccggaaggcggaggacccggagccggggcggtgccggcggacggacggcaagaagtggcgctgctccaaggaggcgtacccggactccaagtactgcgagaagcacatgcaccgcggcaagaaccgttcaagaaagcctgtggaaatgtccttggccacgcccccgccgccgccttcctcctcggcctcctcctcctcctccaacgtccactccgccgtcaacgtcgccaccaccacctcctcccccgcgccgtcctaccaccgccacgccgccgcgactcacgacacgacgccctaccacgcgctctacggcggcccctactcctccgccggccgccagcagcacgctagcgcctaccaccacgccgcgcaggtcagcccgttccacctgcacctcgacaccacccacccgcacccgccgccgtcctactactccagcatggaccacagcaaggacagctacgcctacgggcacagcgtcaaggaggtgcacggcggcggcgagcacgccttcttctcctccgacgtcaccaccgacagggaccatcaccaccaccaccatcagcaccaacaccacgctagcgccggcggcaacggccagtggcagttcaagcagctcggcggcatggagccgaagcagcataacccaacgtcgctcttccccggctgcggcggctacggcaacaacgcggcctacgccatcgacctgtccagcaaagaagaggacgaggagaaggagaggcggcagcagcagcagcactgcttcctgctgggcgccgacctgaggctcgacaagccgtcgtcggggcacggcgactccgccgaccagaagcctctccggcccttcttcgacgagtggccgcacgagaagaccgggagcaaggggtcgtggatggggctcgagggggagacgcagctctccatctccatcgccaacgaactccccatcaccaccacctcccgctaccaccatggtgaatga

SEQ ID NO: 165 - DNA, 1221 bp, Triticum aestivum
  source 1..1221: mol_type = other DNA
atggcgatgccgtatgcctctctttccccggcaggcgaccgccgctcctccccggccgccaccgcctccctcctccccttctgccgctcctccccgttctccgccggcaatggcggcatgggggaggaggcgcggatggccggtaggtggatggcgaggccggcgcccttcacggcggcgcagtacgaggagctggagcaccaggcgctgatatacaagtacctggtggccggcgtgcccgtcccgccggatctcgtgctccccatccgccgcggcatcgagaccctcgccgcccgcttctaccacaaccccctcgccatcgggtatggatcgtacctgggcaagaaggtggatccggagcccggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgcccaacacgttctcctccgcgctggggcctcctcagcagcacatgggcaacaatgcctcctcaccctacgcggctctcggtggcgccggaacatgcaaagatttcaggtataccgcatatggaataagatctttggcagacgagcacagtcagctcatgacagaagccatgaatacctccgtggagaacccatggcgcctgccgccatcgtctcaaacgaccacattcccgctctcaagctacgctcctcagcttggagcaactagtgacctgggtcagaacaacaacagcagcagcagcaacagtgccgtcaagtccgaacggcagcagcagcagcagcccctctccttcccggggtgcggcgacttcggcggcggcggcgccatggactccgcgaagcaggagaaccagacgctgcggccgttcttcgacgagtggcccaagacgagggactcgtggtcggacctgaccgacgacaactccagcctcgcctccttctcggccacccagctgtcgatctcgatacccatgacgtcctccgacttctcggccgccagctcccagtcgcccaacggtatgctgttcgccggcgaaatgtactag

SEQ ID NO: 166 - DNA, 1116 bp, Triticum aestivum
  source 1..1116: mol_type = other DNA
atgctgagctcgtcggcggcgatggggatggggctggggggctacggccagcagcagcagcagcagatgcagatgcagatgcagcggggcgcggggccggtgttcacgccggcgcagtgggccgagctggagcagcaggcgctgatttacaagtacctcatggcaggcgtgcccgtgccgcccgatctcctgctccccatccgcccccaccaccccgccgccggcgccgccggaaccaccttctccttcgccagccccgccgcctcgcccttctaccaccaccaccatccctccatgagttactacgcctactatggcaagaagctcgacccggagccgtggcggtgccggcgcaccgacggcaagaagtggcggtgctccaaggaggcgcaccccgactccaagtactgcgagcgccacatgcaccgtggccgcaaccgttcaagaaagcctgtggaatccaagtctgcttcccctgcgcaccagtcgcagcagcccccgctgtccgccgtcacgtccgcggcccgcgacgccgagccgctcccctccctcccggctggggctaaaacccatggcctgtccctcggcggggctggctcgtcgcagatgcacgtcgacgcctcatcatacggcggcaaatactcccttggagctaaatctgatgtgggtgaactgagcttcttctctggagcatcaggaaacaacaacaggggcttcaccatcgattccccaacggacagctcgtggcactcgatggggtccagcctgcccccgtaccaactgtcgaaacctagagattccggcctcatgcaaggcggcttctcgtattcccactatgagccgtcgcaggagcttgggcaggtaaccatcgcctcgctgtcccactcccaggagcaggactgccgctctttgggtggtggaggtggaggaggtggaggtggaggtggagggctcatgggaaatgtcaagcaggagaaccagccgctgaggcccttcttcgacgagtggccggggaggcgggactcgtggtcggagatggacgacgagcgctccaacggcacctccttctcgacgacccagctctcgatctccatcccgatgcctcgatgcgattga

SEQ ID NO: 167 - DNA, 1155 bp, Triticum aestivum
  source 1..1155: mol_type = other DNA
atggcgatgccctttgcctccctgtcgccggcagccgaccaccaccgctcctcccccatcttccccttctgccgctcctcccctctctactcggcaggggaggaggcggcgcagcagcagcagcagcagcagcacgcgatgagcggcgcgaggtgggcggcggcgaggccggcgaccttcacggcggcgcagtacgaggagctggagcagcaggcgctcatctacaagtacctcgtcgccggcgtgcccgtcccgccggatctcctcctccccatccgccggggcttcgactccctcgcctcgcgcttctaccaccaccacgcccttgggtacgggtcctacttcgggaagaagctggatccggagccggggcggtgccggcggacggacggcaagaagtggcggtgctccaaggaggccgcccaggactccaagtactgcgagcgccacatgcaccgcggccgcaaccgttcaagaaagcctgtggaaacgcagctcgtctcccactcccagcagctgcagcagcaggcccccgccgccgcgttccacggccactcgccgtacccggcgatcgccactggcgccggcgcgcccggctccttcgccctggggtctactgctcagctgcacatggataatgctgctgcgccttacgcgaccgctggcgccgccgggaacaaagatttcaggtattctgcctatgggtttaggacttcggcgatggaggaccacaaccagttcatcagtgcggccatggacaccgccatggacaactactcatggcgcctgctgccggcccagaactcgtccttctcactctcgagctaccccatgctgagcaccctgagcgacctggaccagagcgcgatctgctcgctggccaagacggagagggagccgctgtccttcttcggcgtgggcggcggcttcgacgacgacgagtcggcggtgaagcaggagaaccagacgctgcggcccttcttcgacgagtggcccaaggacagggactcgtggccggagctgcaggaccatgactccaaccacaacaatgaggccttctcggccaccaagctgtccatctccatcccggtgaccagctccgatttctccaccaccgccggctcccgctcgccccacggtatatactcccggtga

SEQ ID NO: 168 - DNA, 639 bp, Triticum aestivum
  source 1..639: mol_type = other DNA
atggccgacgagaaagaagccgactcgctgcagccgccgtccaagcagccccgcctctcctccgccgactcgaacgccggggcggtgacgatggcggtctcgtcgccgctgggtcttggcctcggcctggggctcggtggcgatagccgtggcgagcagcaggccttcgaagcacgggcggcggcgaagtcggcgctgacgttcatgcagcagcaggagctggagcaccaggtgctcatctaccgctacttcgcggcgggggcgcccgtgccggtgcacctcgtgctgcccatctggaagagcgtcgccgcctcctccttcggcccgcaccgcttcccctccctgattgggctggggagcctgtgcttcgactaccggagcagcatggagccggagcccgggcggtgccgccgcacggacggcaagaagtggcggtgctcccgcgacgtggtgcaggggcacaagtactgcgagcggcacgtccaccggggccgcggccgttcaagaaagcctgtggaaggagccccctcagcccgggcgcacagcgacagcaccgccaccgccccgccctgcgccatcggcttctcccccgccggcatcctccacgccacccacagcgccgccgcgcgcgccacctga

SEQ ID NO: 169 - DNA, 951 bp, Triticum aestivum
  source 1..951: mol_type = other DNA
atgatgctgggagggcacggcggcggcggcggcggcgggaggtgcctgttcacggcgtcgcagtggcgggagctggagcaccaggcgctcatctacaagtacatggccgccggctcgcaggtgccccacgagctggtcctcccgctccgccaccgcgacgcagccttcgccgccatcgacaccgccccctccctcgcctgctaccctcctccgcagccatccctggggtgggggctctacggggcgggatcgcagtacgcgcggaagccggaggacccggagcccgggcggtgccggcggacggacggcaagaaatggcggtgctccagggaggcgtatggggagtccaagtactgcgacaggcacatgcaccgcggcaagaaccgttcaagaaagcctgtggaaccaatgagctccgcctcctccgtctcctccccggccgcctcgtaccgccacaccgccctctccatgtcgccccccacgccggccgacacgcccagctacggccacggccacggccacgaccacctccgcgcagctgctggtcagagccagataaaccctctccagctccacctcgacaccccgtcgcccccgccgtcctaccacaggtacgcgccggcgcagcagtacgggggctccttcttcccgagcaggcagcaggtgcaggaggaggaggcgaggcggcggcagcacttcctggctctcggcgccgacctgagcctggacaagccggacgccaccaccgcggcgtcctcgacaaccgaggagaagccgctgcggcgcttcttcgacgagtggccgcgcgacgggaacgccgtcgagggtaggccctggaatatgggccaccgggacgagacgctgctctccatgtccatccccacgacgacggcctcgcaccccgacctcgccgccgcctcgcgctaccaccaccaccacaacgatgaataa

SEQ ID NO: 170 - DNA, 1227 bp, Triticum aestivum
  source 1..1227: mol_type = other DNA
atggcgatgccgtatgcctctctttccccggcaggcgaccgccgctcctccccggccgccaccgccaccgcctccctcctccccttctgccgctcctcccccttctccgccggcggcaatggcggcatgggggaggaggcgccgatggacgggaggtggatggcgaggccggtgcccttcacggcggcgcagtacgaggagctggagcaccaggcgctcatatacaagtacctggtggccggcgtgcccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtactgcgagcgccacatgcaccgcggccgcaaccgttcaagaaagcctgtggaaacgcagctcgtgccccactcccagccgccggccgcctccgccgtgccgcccctcgccaccggcttccacggccactccctctaccccgccgtcggcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagatgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagcgacagcagcagcagcagccgctctccttcccggggtgcggcgacttcggcggcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacctatgacgtcccccgacttctccgccgccagctcccagtcgcccaacggcatgctgttcgccggcgagatgtactag

SEQ ID NO: 171 - DNA, 1230 bp, Triticum aestivum
  source 1..1230: mol_type = other DNA
atgatgatgatgggcggtcgcgcgggggccggcggcgtcggggcaggcggcggccggtgcccgttcacggcgacgcagtggcaggagctggagcaccaggcgctcatctacaagtacatggcctccggcgtgcccatcccctccgacctcctcctcccgctccgccgcagcttcctcctcgactccgccctcgccacctccccctccctcgccttccctccccaggccgcacttgggtggggttgctttggcatggggttcggccggaaggcggaggacccggagccggggcggtgccggcggacggacgggaagaagtggcgctgctccaaggaggcgtacccggactccaagtactgcgagaagcacatgcaccggggcaagaaccgttcaagaaagcctgtggaaatgtccttggccacgcccccgccgccgccttcctcctcggcctcctcttcctcctccaacgtccactccgccgtcaacgtcgccaccaccaccacctccccagcgccgtcctaccaccgccacgccgctgcgactcacgacacgacgccctaccacgccctctacggcggcccctactcctccgccggccgccagcagcacgccagcgcctaccaccacgcggcgcaggtcagcccgttccacctgcacctcgacaccacccacccgcacccgccgccgtcctactactccaccatggaccacagcaaggacagctacgcctacgggcacagcgtcaaggaggtgcacggcggcggcgagcacgccttcttctcctccgacgtcagcaccgacagggaccaccaccaccatcagcaccaacaccacgctagcgccggcggcaacggccagtggcagttcaagcagctcggcggcatggagcccaagcagcacaaccccacgtcgctcttccccggctacggcaacaacgcggcgtacgccatcgacctgtccagcaaagaagaggacgaggagaaggagaggcggcaacagcagcagcactgcttcctgctgggcgccgacctgaggctcgacaagccgtcgtcggggcacggcgactccgccgaccagaagcctctccggccgttcttcgacgagtggccgcacgagaagactggcagcaaggggtcgtggatggggctcgagggggagacgcagctctccatctccatcgccaatgaactccccatcaccaccacctcccgctaccaccatggtgaatga

SEQ ID NO: 172 - DNA, 636 bp, Triticum aestivum
  source 1..636: mol_type = other DNA
atggccgacgagaaagaagccgactcgctgcagccgccgtccaagcagccccgcctctcctccgccgactcgaacgccggggcggtgacgacggcggtctcgtcgccgctgggtcttggcctcggcctggggctcggcggcgatagccgtggcgagcagcaggccttcgaagcacgggcagccgcggcggcggcgaaatcggcgctgacgttcatgcagcagcaggagctggagcaccaggtgctcatctaccgctacttcgcggcgggtgcgcccgtgccggtgcacctcgtgctccccatctggaagagcgtcgccgcctcctccttcggcccgcaccgcttcccctccctgattgggctggggagcctgtgcttcgactaccggagcagcatggagccggagcccgggcggtgccgccgcacggacggcaagaagtggcggtgctcccgcgacgtggtgcaggggcacaagtactgcgagcggcacgtccaccggggccgcggccgttcaagaaagcctgtggaaggagcctccgcagccccggcgcacagcggcagccccaccacagccccgccccgcgccatcggcttctcccccgccggcatcctccacgccacccacagcgccacctga

SEQ ID NO: 173 - DNA, 62 bp, Triticum aestivum
  source 1..62: mol_type = other DNA
cacgtccaccggggccgcggccgttcaagaaagcctgtggaaggagcctccgcagccccggc

SEQ ID NO: 174 - DNA, 62 bp, Triticum aestivum
  source 1..62: mol_type = other DNA
cacatgcaccggggcaagaaccgttcaagaaagcctgtggaaatgtccttggccacgccccc

SEQ ID NO: 175 - DNA, 62 bp, Triticum aestivum
  source 1..62: mol_type = other DNA
cacatgcaccgcggccgcaaccgttcaagaaagcctgtggaaacgcagctcgtgccccactc

SEQ ID NO: 176 - DNA, 62 bp, Triticum aestivum
  source 1..62: mol_type = other DNA
cacatgcaccgcggcaagaaccgttcaagaaagcctgtggaaccaatgagctccgcctcctc

SEQ ID NO: 177 - DNA, 62 bp, Triticum aestivum
  source 1..62: mol_type = other DNA
cacgtccaccggggccgcggccgttcaagaaagcctgtggaaggagccccctcagcccgggc

SEQ ID NO: 178 - DNA, 62 bp, Triticum aestivum
  source 1..62: mol_type = other DNA
cacatgcaccgcggccgcaaccgttcaagaaagcctgtggaaacgcagctcgtctcccactc

SEQ ID NO: 179 - DNA, 62 bp, Triticum aestivum
  source 1..62: mol_type = other DNA
cacatgcaccgtggccgcaaccgttcaagaaagcctgtggaatccaagtctgcttcccctgc

SEQ ID NO: 180 - DNA, 62 bp, Triticum aestivum
  source 1..62: mol_type = other DNA
cacatgcaccgcggccgcaaccgttcaagaaagcctgtggaaacgcagctcgtctcgcactc

SEQ ID NO: 181 - DNA, 62 bp, Triticum aestivum
  source 1..62: mol_type = other DNA
cacatgcaccgcggcaagaaccgttcaagaaagcctgtggaaatgtccttggccacgccccc

SEQ ID NO: 182 - DNA, 62 bp, Triticum aestivum
  source 1..62: mol_type = other DNA
cacatgcaccgcggcaagaaccgttcaagaaagcctgtggaaccgatgtcctcctcctccgt

SEQ ID NO: 183 - DNA, 62 bp, Triticum aestivum
  source 1..62: mol_type = other DNA
cacgtccaccggggccgcggccgttcaagaaagcctgtggaaggagcccccgcagccccggc

SEQ ID NO: 184 - DNA, 62 bp, Triticum aestivum
  source 1..62: mol_type = other DNA
cacgtccaccgcggccgcggccgtgcaagaaagcctgtggaagccgcgccggccacatcagc

SEQ ID NO: 185 - DNA, 62 bp, Triticum aestivum
  source 1..62: mol_type = other DNA
cacatgcaccgcggccgcaaccgttcaagaaagcctgtggaaacgcagctcgtcccgcacac

SEQ ID NO: 186 - DNA, 62 bp, Triticum aestivum; identical to SEQ ID NO: 181
  source 1..62: mol_type = other DNA

SEQ ID NO: 187 - DNA, 62 bp, Triticum aestivum
  source 1..62: mol_type = other DNA
cacatgcaccgcggcaagaaccgttcaagaaagcctgtggaaccgatgagctcctcctccgt

SEQ ID NO: 188 - DNA, 62 bp, Triticum aestivum
  source 1..62: mol_type = other DNA
catatgaaccggggacgccatcgttcaagaaagcatgtggaaggccagcctggccatgccgc

SEQ ID NO: 189 - DNA, 62 bp, Triticum aestivum
  source 1..62: mol_type = other DNA
cacatcaataggaaccgccatcgttcaagaaagcctgtggaaaaccaaacaaggaagaacgc

SEQ ID NO: 190 - DNA, 62 bp, Triticum aestivum
  source 1..62: mol_type = other DNA
cacatgcaccgcggccgcaaccgttcaagaaagcctgtggaaacgcagctcgtcgccacgcc

SEQ ID NO: 191 - DNA, 62 bp, Triticum aestivum; identical to SEQ ID NO: 189
  source 1..62: mol_type = other DNA

SEQ ID NO: 192 - DNA, 62 bp, Triticum aestivum
  source 1..62: mol_type = other DNA
cacgtccaccgcggccgcggccgtgcaagaaagcctgtggaagccgcggcggccacatcagc

SEQ ID NO: 193 - DNA, 62 bp, Triticum aestivum; identical to SEQ ID NO: 188
  source 1..62: mol_type = other DNA

SEQ ID NO: 194 - DNA, 62 bp, Triticum aestivum; identical to SEQ ID NO: 190
  source 1..62: mol_type = other DNA

SEQ ID NO: 195 - DNA, 62 bp, Triticum aestivum
  source 1..62: mol_type = other DNA
cacgtccaccgcggccgcagccgtgcaagaaagcctgtggaagccgcggcggccacatcagc

SEQ ID NO: 196 - DNA, 62 bp, Triticum aestivum; identical to SEQ ID NO: 188
  source 1..62: mol_type = other DNA

SEQ ID NO: 197 - DNA, 62 bp, Triticum aestivum; identical to SEQ ID NO: 179
  source 1..62: mol_type = other DNA

SEQ ID NO: 198 - DNA, 62 bp, Triticum aestivum; identical to SEQ ID NO: 179
  source 1..62: mol_type = other DNA

SEQ ID NO: 199 - DNA, 22 bp, Triticum aestivum
  source 1..22: mol_type = genomic DNA
ccgttcaagaaagcctgtggaa

SEQ ID NO: 200 - DNA, 22 bp, Triticum aestivum
  source 1..22: mol_type = genomic DNA
ccgtgcaagaaagcctgtggaa

SEQ ID NO: 201 - DNA, 22 bp, Triticum aestivum
  source 1..22: mol_type = genomic DNA
tcgttcaagaaagcatgtggaa

SEQ ID NO: 202 - DNA, 22 bp, Triticum aestivum
  source 1..22: mol_type = genomic DNA
tcgttcaagaaagcctgtggaa

SEQ ID NO: 203-205 - skipped (3 entries)

SEQ ID NO: 206 - protein, 207 aa, synthetic construct
  REGION 1..207: note = Synthetic cytosine deaminase
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 207 - protein, 228 aa, synthetic construct
  REGION 1..228: note = Synthetic cytosine deaminase
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 208 - protein, 162 aa, synthetic construct
  REGION 1..162: note = Synthetic cytosine deaminase
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 209 - protein, 1763 aa, synthetic construct
  REGION 1..1763: note = Synthetic adenosine deaminase
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 210 - protein, 1565 aa, synthetic construct
  REGION 1..1565: note = Synthetic adenosine deaminase
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 211 - protein, 1565 aa, synthetic construct
  REGION 1..1565: note = Synthetic adenosine deaminase
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 212 - protein, 364 aa, synthetic construct
  REGION 1..364: note = Synthetic adenosine deaminase
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 213 - protein, 167 aa, synthetic construct
  REGION 1..167: note = Synthetic adenosine deaminase
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 214 - protein, 1228 aa, unidentified
  REGION 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 215 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 216 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 217 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 218 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 219 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 220 - protein, 1206 aa, unidentified
  REGION 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 221 - protein, 1233 aa, unidentified
  REGION 1..1233: note = Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 222 - protein, 1227 aa, unidentified
  REGION 1..1227: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 223 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 224 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 225 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 226 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 227 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 228 - protein, 1484 aa, unidentified
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 229 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 230 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 231 - protein, 3987 aa, synthetic construct
  REGION 1..3987: note = Synthetic Cas12a
ATGGCCGGGAGCAAGAAGCGCCGGATAAAGCAGGACACGCAGTTCGAGGGCTTCACCAACCTGTACCAAGTCTCCAAGACGCTCCGGTTCGAGCTTATCCCGCAAGGGAAGACCCTGAAACACATCCAGGAACAAGGTTTCATCGAGGAGGACAAGGCCCGCAACGACCACTACAAGGAGCTCAAGCCCATAATCGATCGGATCTACAAGACGTACGCCGACCAGTGCCTCCAACTGGTGCAGCTCGACTGGGAGAACCTGAGCGCCGCCATTGACAGCTACCGCAAGGAAAAGACGGAGGAGACGCGCAACGCCCTTATTGAGGAGCAAGCCACCTACCGCAACGCCATCCACGACTACTTCATCGGGCGCACCGACAACCTGACGGACGCGATCAACAAGCGCCACGCGGAAATCTACAAGGGCCTTTTCAAGGCCGAGCTCTTCAACGGGAAGGTCCTAAAACAGCTCGGGACTGTCACGACAACCGAGCATGAGAACGCCCTCCTTCGCAGCTTCGACAAGTTCACCACATACTTCTCGGGCTTCTACCGGAACCGCAAGAACGTTTTCAGCGCCGAGGACATCTCCACCGCCATCCCGCACAGGATCGTCCAGGACAACTTCCCCAAGTTCAAGGAGAACTGCCACATCTTCACGCGCCTGATTACAGCCGTACCTTCACTTCGTGAGCACTTCGAGAACGTCAAAAAGGCCATCGGGATCTTCGTCTCCACGTCCATCGAGGAGGTATTCTCTTTCCCGTTCTATAACCAGCTCCTGACCCAGACGCAGATCGACCTCTACAACCAGCTACTGGGCGGCATCAGCCGGGAGGCCGGGACCGAGAAAATAAAGGGCCTCAACGAAGTTCTCAACCTGGCCATCCAGAAGAACGACGAGACCGCGCATATCATCGCATCCCTGCCGCATCGCTTCATTCCTTTGTTCAAGCAGATATTGAGCGACCGGAACACCCTCTCGTTCATCCTCGAAGAATTCAAGAGCGACGAGGAGGTCATTCAGTCTTTCTGCAAGTACAAGACGCTCCTACGGAATGAGAATGTGCTGGAGACCGCGGAGGCACTCTTCAATGAGCTGAACTCCATTGACCTGACCCACATCTTCATTAGCCACAAGAAACTGGAGACGATCTCCAGCGCCCTGTGCGACCACTGGGACACTCTCCGCAACGCCCTCTACGAACGCCGGATCTCCGAACTTACCGGCAAGATAACTAAGTCGGCTAAGGAGAAGGTGCAACGGAGCCTCAAGCACGAGGACATCAACCTTCAGGAAATCATCTCAGCCGCGGGCAAGGAGCTGAGCGAGGCGTTTAAGCAGAAAACATCGGAGATACTGAGCCACGCGCACGCGGCCCTGGATCAACCGCTGCCGACGACTCTCAAGAAGCAAGAGGAGAAGGAAATCCTTAAGTCCCAGCTCGACTCGCTGCTCGGCCTCTATCACTTGCTCGACTGGTTCGCGGTTGATGAGTCCAACGAGGTGGACCCGGAGTTCTCCGCGCGCCTCACGGGTATTAAGCTGGAGATGGAGCCAAGCTTAAGCTTCTACAACAAGGCCCGCAACTACGCGACCAAAAAACCGTACTCAGTCGAGAAATTCAAGCTGAATTTCCAGATGCCTACATTGGCGAGGGGGTGGGACGTGAACCGCGAGAAGAACAATGGAGCCATCCTGTTCGTCAAAAATGGGTTGTACTACCTGGGCATCATGCCCAAGCAGAAGGGCCGTTACAAGGCCCTGTCATTCGAGCCTACCGAGAAGACCTCGGAGGGCTTCGACAAGATGTACTACGACTATTTCCCGGACGCCGCCAAGATGATCCCGAAGTGCTCCACGCAGCTCAAAGCCGTCACGGCCCACTTCCAGACGCATACCACGCCGATACTTCTGAGCAACAACTTCATTGAGCCGCTAGAGATCACGAAGGAGATATACGACCTAAACAACCCCGAAAAGGAGCCCAAGAAGTTCCAGACAGCCTACGCTAAGAAGACAGGTGATCAGAAGGGATATAGGGAGGCACTCTGCAAGTGGATCGACTTCACGCGCGACTTCCTGTCGAAATATACAAAGACGACCAGCATTGACCTAAGTTCTCTCCGCCCATCCTCCCAGTACAAGGATCTGGGCGAGTATTATGCGGAGCTGAACCCATTGCTGTACCACATCAGCTTCCAGAGGATCGCCGAGAAGGAGATTATGGACGCGGTGGAGACGGGGAAACTATACCTGTTCCAAATATATAACAAGGACTTCGCTAAAGGGCACCACGGGAAGCCCAACCTGCACACACTCTACTGGACGGGCTTGTTTTCGCCAGAAAATTTGGCCAAGACTTCGATCAAGCTCAACGGCCAGGCGGAGTTGTTTTACCGTCCCAAGTCTCGCATGAAGCGCATGGCGCATCGCCTCGGAGAGAAAATGCTTAACAAGAAGCTCAAGGATCAGAAGACGCCCATACCTGATACGTTGTACCAGGAATTGTACGACTACGTGAACCACCGCCTATCGCACGACCTCTCAGACGAGGCCCGCGCCCTCCTCCCAAACGTGATTACTAAGGAGGTTTCCCATGAAATAATCAAGGACCGACGGTTCACCAGCGACAAATTTTTTTTCCACGTGCCTATCACGCTCAATTACCAGGCGGCCAACTCCCCATCGAAGTTCAACCAGCGCGTGAACGCCTACCTTAAGGAGCACCCGGAGACCCCAATCATCGGGATCGACCGTGGCGAGCGGAACCTGATCTATATTACGGTGATCGATAGCACCGGGAAGATCCTGGAGCAGCGCTCCCTGAACACAATCCAGCAGTTTGACTACCAGAAGAAACTCGACAACCGGGAGAAGGAGCGCGTCGCAGCCCGGCAAGCATGGAGTGTGGTCGGCACCATAAAGGACCTGAAACAGGGTTACCTAAGTCAAGTTATCCACGAGATCGTTGACCTGATGATACACTATCAAGCCGTAGTCGTGCTGGAGAACCTCAACTTCGGGTTTAAGTCCAAGCGCACCGGCATCGCGGAGAAGGCGGTGTACCAGCAGTTCGAGAAGATGCTGATCGACAAGCTGAACTGCCTGGTGCTCAAGGACTACCCTGCGGAGAAGGTCGGCGGGGTCTTGAACCCGTACCAGCTAACCGACCAGTTCACGAGCTTCGCCAAAATGGGCACGCAGTCCGGATTCTTGTTTTATGTCCCGGCTCCATATACAAGTAAGATCGACCCGCTGACAGGGTTTGTTGACCCATTCGTGTGGAAGACCATCAAGAACCACGAGAGCAGGAAACACTTCTTAGAGGGCTTCGACTTCCTGCATTACGACGTTAAGACAGGCGACTTCATCCTGCACTTCAAGATGAACCGCAACCTGTCGTTCCAGAGGGGCCTGCCCGGCTTCATGCCCGCCTGGGATATCGTCTTTGAGAAGAATGAGACGCAGTTCGACGCGAAGGGGACGCCGTTCATCGCTGGAAAGCGGATCGTGCCGGTCATCGAGAACCACCGCTTCACGGGTCGCTACCGAGATTTATACCCCGCCAACGAACTAATTGCGCTGCTGGAGGAGAAGGGGATCGTGTTCCGAGATGGCAGCAACATTCTCCCGAAGCTGCTGGAGAACGACGACTCGCACGCTATTGACACGATGGTCGCCCTCATACGGAGCGTGCTTCAGATGCGGAACAGTAACGCTGCCACGGGCGAGGACTACATTAACTCCCCCGTCCGCGACCTCAACGGGGTCTGCTTCGATAGCCGCTTCCAGAACCCGGAGTGGCCTATGGATGCGGACGCGAACGGGGCCTACCACATCGCCCTCAAGGGCCAACTCCTGCTCAACCACTTGAAGGAAAGCAAAGACCTCAAATTGCAGAATGGCATCAGTAACCAGGACTGGCTCGCGTACATCCAGGAACTGAGAAACGGGTCCAAGAAGCGGCGTATCAAGCAAGATTGA

SEQ ID NO: 232 - DNA, 3987 bp, synthetic construct
  misc_feature 1..3987: note = Synthetic Cas12a
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 233 - DNA, 3987 bp, synthetic construct
  misc_feature 1..3987: note = Synthetic Cas12a
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 234 - protein, 1367 aa, synthetic construct
  REGION 1..1367: note = Synthetic nCas9
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 235 - protein, 1367 aa, synthetic construct
  REGION 1..1367: note = Synthetic enCas9
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 236 - DNA, 4101 bp, unidentified
  misc_feature 1..4101: note = Cas9 nucleotide sequence
  source 1..4101: mol_type = genomic DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 237 - DNA, 4101 bp, unidentified
  misc_feature 1..4101: note = Cas9 nucleotide sequence
  source 1..4101: mol_type = genomic DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 238 - DNA, 4092 bp, unidentified
  misc_feature 1..4092: note = Cas9 nucleotide sequence
  source 1..4092: mol_type = genomic DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 239 - DNA, 4101 bp, unidentified
  misc_feature 1..4101: note = Cas9 nucleotide sequence
  source 1..4101: mol_type = genomic DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 240 - DNA, 4101 bp, unidentified
  misc_feature 1..4101: note = Cas9 nucleotide sequence
  source 1..4101: mol_type = genomic DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 241 - DNA, 4092 bp, unidentified
  misc_feature 1..4092: note = Cas9 nucleotide sequence
  source 1..4092: mol_type = genomic DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 242 - DNA, 4101 bp, unidentified
  misc_feature 1..4101: note = Cas9 nucleotide sequence
  source 1..4101: mol_type = genomic DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 243 - DNA, 4101 bp, unidentified
  misc_feature 1..4101: note = Cas9 nucleotide sequence
  source 1..4101: mol_type = genomic DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 244 - DNA, 4101 bp, unidentified
  misc_feature 1..4101: note = Cas9 nucleotide sequence
  source 1..4101: mol_type = genomic DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 245 - DNA, 3307 bp, unidentified
  misc_feature 1..3307: note = Cas9 nucleotide sequence
  source 1..3307: mol_type = genomic DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 246 - DNA, 4101 bp, unidentified
  misc_feature 1..4101: note = Cas9 nucleotide sequence
  source 1..4101: mol_type = genomic DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 247 - DNA, 4101 bp, unidentified
  misc_feature 1..4101: note = Cas9 nucleotide sequence
  source 1..4101: mol_type = genomic DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 248 - DNA, 4101 bp, unidentified
  misc_feature 1..4101: note = Cas9 nucleotide sequence
  source 1..4101: mol_type = genomic DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggccgacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgccctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcccctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 249 - DNA, 4101 bp, unidentified
  misc_feature 1..4101: note = Cas9 nucleotide sequence
  source 1..4101: mol_type = genomic DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 250 - DNA, 4101 bp, unidentified
  misc_feature 1..4101: note = Cas9 nucleotide sequence
  source 1..4101: mol_type = genomic DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 251 - DNA, 23 bp, synthetic construct
  misc_feature 1..23: note = Synthetic spacer
  source 1..23: mol_type = other DNA
tgattccacaggctttcttgaac

SEQ ID NO: 252 - DNA, 26 bp, synthetic construct
  misc_feature 1..26: note = Synthetic binding site of GRF5
  source 1..26: mol_type = other DNA
aaccgttcaagaaagcctgtggaacg

SEQ ID NO: 253 - protein, 8 aa, Zea mays
NRSRKPVE

SEQ ID NO: 254 - DNA, 27 bp, Zea mays
  source 1..27: mol_type = genomic DNA
aaccgttcaagaaagcctgtggaaacg

SEQ ID NO: 255 - protein, 9 aa, synthetic construct
  REGION 1..9: note = Mutant GRF miRNA binding site
NRSRKPLKT

SEQ ID NO: 256 - protein, 9 aa, synthetic construct
  REGION 1..9: note = Mutant GRF miRNA binding site
NRSKKPVET

SEQ ID NO: 257-259 - skipped (3 entries)

SEQ ID NO: 260 - protein, 20 aa, Zea mays
HMHRGRNRSRKPVETQLAPQ

SEQ ID NO: 261 - DNA, 20 bp, synthetic construct
  misc_feature 1..20: note = Synthetic spacer
  source 1..20: mol_type = other DNA
cttccacaggctttcttgaa

SEQ ID NO: 262 - DNA, 20 bp, synthetic construct
  misc_feature 1..20: note = Synthetic spacer
  source 1..20: mol_type = other DNA
attccacaggctttcttgaa

SEQ ID NO: 263 - DNA, 27 bp, synthetic construct
  misc_feature 1..27: note = Synthetic binding site of GRF6
  source 1..27: mol_type = other DNA
aaccgttcaagaaagcctgtggaaacc

SEQ ID NO: 264 - DNA, 27 bp, synthetic construct
  misc_feature 1..27: note = Synthetic binding site of GRF6
  source 1..27: mol_type = other DNA
aaccgttcaagaaaacctgtggaaacc

SEQ ID NO: 265 - DNA, 27 bp, synthetic construct
  misc_feature 1..27: note = Synthetic binding site of GRF6
  source 1..27: mol_type = other DNA
aaccgttcaagaaagcctgtcaaaacc

SEQ ID NO: 266 - DNA, 27 bp, synthetic construct
  misc_feature 1..27: note = Synthetic binding site of GRF6
  source 1..27: mol_type = other DNA
aaccgttcaagaaaacctgtcaaaacc

SEQ ID NO: 267 - DNA, 27 bp, synthetic construct
  misc_feature 1..27: note = Synthetic binding site of GRF6
  source 1..27: mol_type = other DNA
aaccgttcaaaaaagcctgtggaaacc

SEQ ID NO: 268 - DNA, 27 bp, synthetic construct
  misc_feature 1..27: note = Synthetic binding site of GRF6
  source 1..27: mol_type = other DNA
aaccgttcaagaaagcctgtaaaaacc

SEQ ID NO: 269 - DNA, 27 bp, synthetic construct
  misc_feature 1..27: note = Synthetic binding site of GRF6
  source 1..27: mol_type = other DNA
aaccgttcaagaaaacctgtaaaaacc

SEQ ID NO: 270 - DNA, 23 bp, synthetic construct
  misc_feature 1..23: note = Synthetic spacer
  source 1..23: mol_type = other DNA
cacaggctttcttgaacggttct

SEQ ID NO: 271 - DNA, 23 bp, synthetic construct
  misc_feature 1..23: note = Synthetic spacer
  source 1..23: mol_type = other DNA
cacaggctttcttgaacggtgac

SEQ ID NO: 272 - DNA, 23 bp, synthetic construct
  misc_feature 1..23: note = Synthetic spacer
  source 1..23: mol_type = other DNA
cacaggctttcttgaacggttct

SEQ ID NO: 273 - DNA, 23 bp, synthetic construct
  misc_feature 1..23: note = Synthetic spacer
  source 1..23: mol_type = other DNA
cacaggctttcttgaacggtggc

SEQ ID NO: 274 - DNA, 23 bp, synthetic construct
  misc_feature 1..23: note = Synthetic spacer
  source 1..23: mol_type = other DNA
cacaggctttcttgaactgtgaa

SEQ ID NO: 275 - DNA, 68 bp, Zea mays
  source 1..68: mol_type = genomic DNA
agtactgcgagcgccacatgcaccgcggccgcaaccgttcaagaaagcctgtggaaacgcagctcgcg

SEQ ID NO: 276 - DNA, 60 bp, synthetic construct
  misc_feature 1..60: note = Synthetic oligonucleotide
  source 1..60: mol_type = other DNA
agtactgcgagcgccacatgcaccgcggccgcaagaaagcctgtggaaacgcagctcgcg

SEQ ID NO: 277 - DNA, 58 bp, synthetic construct
  misc_feature 1..58: note = Synthetic oligonucleotide
  source 1..58: mol_type = other DNA
agtactgcgagcgccacatgcaccgcggccgagaaagcctgtggaaacgcagctcgcg

SEQ ID NO: 278 - DNA, 23 bp, synthetic construct
  misc_feature 1..23: note = Synthetic oligonucleotide
  source 1..23: mol_type = other DNA
agcctgtggaaacgcagctcgcg

SEQ ID NO: 279 - DNA, 62 bp, synthetic construct
  misc_feature 1..62: note = Synthetic oligonucleotide
  source 1..62: mol_type = other DNA
agtactgcgagcgccacatgcaccgcggccgctcaagaaagcctgtggaaacgcagctcgcg

SEQ ID NO: 280 - DNA, 59 bp, synthetic construct
  misc_feature 1..59: note = Synthetic oligonucleotide
  source 1..59: mol_type = other DNA
agtactgcgagcgccacatgcaccgcggcccaagaaagcctgtggaaacgcagctcgcg

SEQ ID NO: 281 - DNA, 27 bp, synthetic construct
  misc_feature 1..27: note = Synthetic oligonucleotide
  source 1..27: mol_type = other DNA
agaaagcctgtggaaacgcagctcgcg

SEQ ID NO: 282 - DNA, 58 bp, synthetic construct
  misc_feature 1..58: note = Synthetic oligonucleotide
  source 1..58: mol_type = other DNA
agtactgcgagcgccacatgcaccgcggccaagaaagcctgtggaaacgcagctcgcg

SEQ ID NO: 283 - DNA, 61 bp, synthetic construct
  misc_feature 1..61: note = Synthetic oligonucleotide
  source 1..61: mol_type = other DNA
agtactgcgagcgccacatgcaccgcggccgtcaagaaagcctgtggaaacgcagctcgcg

SEQ ID NO: 284 - DNA, 60 bp, synthetic construct; identical to SEQ ID NO: 276
  misc_feature 1..60: note = Synthetic oligonucleotide
  source 1..60: mol_type = other DNA

SEQ ID NO: 285 - DNA, 56 bp, synthetic construct
  misc_feature 1..56: note = Synthetic oligonucleotide
  source 1..56: mol_type = other DNA
agtactgcgagcgccacatgcaccgcggccgaaagcctgtggaaacgcagctcgcg

SEQ ID NO: 286 - DNA, 60 bp, synthetic construct; identical to SEQ ID NO: 276
  misc_feature 1..60: note = Synthetic oligonucleotide
  source 1..60: mol_type = other DNA

SEQ ID NO: 287 - DNA, 40 bp, synthetic construct
  misc_feature 1..40: note = Synthetic oligonucleotide
  source 1..40: mol_type = other DNA
agtactgcgagcgccacatgcaccgcggccacggctcgcg

SEQ ID NO: 288 - DNA, 33 bp, synthetic construct
  misc_feature 1..33: note = Synthetic oligonucleotide
  source 1..33: mol_type = other DNA
agtactgcgagcgccacatgcaccgcggccgcg

SEQ ID NO: 289 - DNA, 59 bp, synthetic construct; identical to SEQ ID NO: 280
  misc_feature 1..59: note = Synthetic oligonucleotide
  source 1..59: mol_type = other DNA

SEQ ID NO: 290 - DNA, 63 bp, synthetic construct
  misc_feature 1..63: note = Synthetic oligonucleotide
  source 1..63: mol_type = other DNA
agtactgcgagcgccacatgcaccgcggccggttcaagaaagcctgtggaaacgcagctcgcg

SEQ ID NO: 291 - DNA, 58 bp, synthetic construct; identical to SEQ ID NO: 282
  misc_feature 1..58: note = Synthetic oligonucleotide
  source 1..58: mol_type = other DNA

SEQ ID NO: 292 - DNA, 67 bp, synthetic construct
  misc_feature 1..67: note = Synthetic oligonucleotide
  source 1..67: mol_type = other DNA
agtactgcgagcgccacatgcaccgcggccgcaaccgtcaagaaagcctgtggaaacgcagctcgcg

SEQ ID NO: 293 - DNA, 63 bp, synthetic construct
  misc_feature 1..63: note = Synthetic oligonucleotide
  source 1..63: mol_type = other DNA
agtactgcgagcgccacatgcaccgcggccgcttcaagaaagcctgtggaaacgcagctcgcg

SEQ ID NO: 294 - DNA, 39 bp, synthetic construct
  misc_feature 1..39: note = Synthetic oligonucleotide
  source 1..39: mol_type = other DNA
agtactgcgagcgccacatgcaccgcggcccagtcccaa

SEQ ID NO: 295 - DNA, 59 bp, synthetic construct; identical to SEQ ID NO: 280
  misc_feature 1..59: note = Synthetic oligonucleotide
  source 1..59: mol_type = other DNA

SEQ ID NO: 296 - DNA, 62 bp, synthetic construct; identical to SEQ ID NO: 279
  misc_feature 1..62: note = Synthetic oligonucleotide
  source 1..62: mol_type = other DNA

SEQ ID NO: 297 - DNA, 59 bp, synthetic construct
  misc_feature 1..59: note = Synthetic oligonucleotide
  source 1..59: mol_type = other DNA
agtactgcgagcgccacatgcaccgcggccgcaagaagcctgtggaaacgcagctcgcg

SEQ ID NO: 298 - DNA, 62 bp, synthetic construct
  misc_feature 1..62: note = Synthetic oligonucleotide
  source 1..62: mol_type = other DNA
agtactgcgagcgccacatgcaccgcggccgcaaccgaaagcctgtggaaacgcagctcgcg

SEQ ID NO: 299 - DNA, 59 bp, synthetic construct; identical to SEQ ID NO: 280
  misc_feature 1..59: note = Synthetic oligonucleotide
  source 1..59: mol_type = other DNA

SEQ ID NO: 300 - DNA, 65 bp, synthetic construct
  misc_feature 1..65: note = Synthetic oligonucleotide
  source 1..65: mol_type = other DNA
agtactgcgagcgccacatgcaccgcggcaaccgttcaagaaagcctgtggaaacgcagctcgcg

SEQ ID NO: 301 - DNA, 63 bp, synthetic construct
  misc_feature 1..63: note = Synthetic oligonucleotide
  source 1..63: mol_type = other DNA
agtactgcgagcgccacatgcaccgcggccgcaacaagaaagcctgtggaaacgcagctcgcg

SEQ ID NO: 302 - DNA, 67 bp, synthetic construct
  misc_feature 1..67: note = Synthetic oligonucleotide
  source 1..67: mol_type = other DNA
agtactgcgagcgccacatgcaccgcggccgcaaccttcaagaaagcctgtggaaacgcagctcgcg

SEQ ID NO: 303 - DNA, 59 bp, synthetic construct; identical to SEQ ID NO: 280
  misc_feature 1..59: note = Synthetic oligonucleotide
  source 1..59: mol_type = other DNA

SEQ ID NO: 304 - DNA, 3431 bp, Zea mays
  source 1..3431: mol_type = genomic DNA
ggagcgaagcaaagcacatcacgagcccagcctgcgcctgcggagggagggggctcattaaagagggggcgcgagcgcgaccggccgcggggagcaagcagcgcgcgagagagacaggttgagatggcgatgccgtatgcctctctttccccggcaggcgccgccgaccaccgctcctccacagccacggcgtccctcgtccccttctgccgctccactccgctctccgcgtaagtatccaccaccgtcgctcatcgcttcttggctcgtgtagtgtcgtctcccattgatgcccgtctggttcgcggttgtgttgtgacgacgtggtggtatgtgtgctggtgcaggggcggcgggctgggcgaggaggacgcccaggcgagcgcgaggtggccggccgcgaggccggtggtgccgttcacgccggcgcagtaccaggagctggagcagcaggcgctcatatacaagtacctggtggcgggcgtgcccgttccgccggatctcgtggttccaatccgccgcggcctcgactccctcgcaacccgcttctacggccaacccacacgtacgtacggcatttccctccctctcctctcggcgccctgccagactccatctccttcctgtactgcgtccggggtgcttggctgcttgctccctccccgggatcctgactctttgtttattccagccaagcgtactttgcctccattagttaatttgctccagtcatttgttaatccctggtgcgcgattcaagaaccgtgcgcgtgtttcatacaagacccccgatttgctcccggaaagtttctgtttttttaatccgagctgattcatcaaatgatgaggcagagctagctaggtagctcgtcatttcgtgctgttttgatttgataccgagcttagtagttgttggaatagaggttagaatcagtagcacgcatcgttgtggctcaatagtacgaatacttttcttttgccatttatttcctctccttctggcttctggatgatcaatactacgtaactgtgagaggtcgccccctcggcagcaagcaaattcattgaatgcaatctgcagcagctctccgctagctgctcgccatttcgtccgactgcgcctgcatcggtactgtcctgtactaccaaatggcaatccatttggacactgcagatccagatgcacgcgtagttgctgcgtacatgcagatgcacgatgataaaagcaaacgcacaagtgatgcttaaatacagcgtctgattttctcggcgtgatctgtgcagtcgggtacggaccgtacctggggaggaaactggatccggagcccggccggtgccggcgaacggacggcaagaagtggcggtgctccaaggaggccgccccggactccaagtactgcgagcgccacatgcaccgcggccgcaaccgttcaagaaagcctgtggaaacgcagctcgcgccccagtcccaaccgcccgccgccgcagccgtctccgccgctccgcccctggcagccgccgccgccgccaccaccaacggcagcggcttccagaaccactctctctacccggccatcgccggcagcactggtggtggaggaggagttggcgggtccggcaatatctcctccccgttctcctcgtcgatggggggatcgtctcagctgcacatggacagtgctgccagctactcctacgcagctcttggtggtggaactgcaaaggatctcaggtgactattctttcccttttcttttatttcctctccagtgaccaatgctatcctgtttttctaacagtgctatcgattttaactgattggcgtcagatggccacacatgctataacattttaccgatgatcgttcactaatctctgtctgtttggcatcagtggcaatcaaaaccagtagcgtagtgcgtgctccgtagtacttcactggcaaaatcagcatggcctacgaggactttactcacttcaagagattacgttagagttcaggcgctggctggttgcctggccacgctttattaatgagttcagttctaaacccctgctgaagcccaggcagtgtccatgtctctacgagatagttgtgcccagggcaggtggacgtgcaggtgtgcagcgcacagccttgttggcttcggcacagacccagcttcgaattcgtgctggtgctgcgcataaagatggaacttgtttgtgtccagcggtcgcagtgctggtgcctgctgcttgctgatgctgtccttccctggccattactccacgtctttatgatctcgcaggatcaagtcgttgctcttgcaactcttttagggggcagcatgtaataaaatccggtccttgtaactactgataaacctgaaacgcatcatgctggaagtccatccccttttgcccataccagtttggttgattttactgtacagatgttctttcactttgtttacaatttatcgaacaggataatgaagactaacacctcacaaagaagaaaacatactgcagttggcatgtgtcctttttaacctttgcaacccccctactaatttgctgtgcccaccccgctttctgaaatccaggtacaacgcttacggaataagatctctggcggacgagcacaaccagctgatcgcagaagccatcgactcgtcgatagagagccagtggcgcctccccagctcgtcgttcccgctctcgagctacccacatctcggggcgctgggcgacctgggcggccagaacagcacggtgagctcgctgccgaagatggagaagcagcagccgccctcgtccttcctagggaacgacaccggggccggcatggccatgggctccgcctccgcgaagcaggagggccagacgctgcggcacttcttcgacgagtggcccaaggcgcgggactcctggccgggcctctccgacgagaccgccagcctcgcctcgttccccccggcgacccagctgtcgatgtccatacccatggcgtcctccgacttctccgtggccagctcccagtcgcccaacggtgagtcgcgtacgttcctgctggccacggaccgaaggtgaaaggttgttgctggtttactgataggagacaaccatgtctggtctctgcagatgactaatggtgcgtggatcgtcgcgttctggccctttgtctgtcttccctccagtcctccacccaccgcgcagtagtagctgcggaaacagcccatgctcctgtatatttgtcggtcattttccgtgtcagatctgtgtaccaaaccaagcggcggcggccgtcgtctctccccgcctccgcctcgcctcatgtgggtgggtgaacatgagaccgttttgcttaggctcaaagctttgtgtgaccccactgatcggttgtctgcgtactacctgaaagcctgctgcttttgtagcattactgt